(19) United States
(12) Patent Application Publication
(10) Pub. No.: US 2022/0177457 A1
(43) Pub. Date: Jun. 9, 2022

(54) SMALL MOLECULES THAT BIND CYCLIN-DEPENDENT KINASE INHIBITOR 1B (P27KIP1)

(71) Applicant: St. Jude Children's Hospital, Inc., Memphis (JP)

(72) Inventors: Richard KRIWACKI, Memphis, TN (US); Jian ZUO, Memphis, TN (US); Luigi ICONARU, Memphis, TN (US); Sourav DAS, Memphis, TN (US); Anang SHELAT, Memphis, TN (US); Brandon YOUNG, Memphis, TN (US)

(21) Appl. No.: 17/438,857

(22) PCT Filed: Mar. 12, 2020
(86) PCT No.: PCT/US2020/022475

§ 371 Date: Sep. 13, 2021

Related U.S. Application Data
(60) Provisional application No. 62/817,924, filed on Mar. 13, 2019.

(51) Int. Cl. C07D 405/12; C07D 405/14; C07D 407/14

(52) U.S. Cl. CPC C07D 405/12; C07D 407/14; C07D 405/14

(57) ABSTRACT

Various compounds and pharmaceutically acceptable salts thereof are provided capable of binding cyclin-dependent kinase inhibitor 1B. The compounds can have a structure according to Formula I or Formula II as detailed herein. The compounds can include SJ747, SJ749, SJ755, SJ757. Pharmaceutical formulations containing the compounds or pharmaceutically acceptable salts are also provided along with methods of use thereof. The formulations and methods can be useful for treating cancer. In some aspects, the cancer is associated with a mislocalization of the intrinsically disordered protein p27. In some aspects, the cancer is resistant to an anticancer therapy. The pharmaceutical formulation can therefore include a second active agent and/or can be given in combination with a second active agent such as a cancer therapeutic. In various aspects, methods of promoting reentry into the cell division cycle in a subject in need thereof using compounds and formulations described herein are also provided.

### CROSS-REFERENCE TO RELATED APPLICATIONS

This application claims priority to, and the benefit of, co-pending U.S. provisional application entitled “SMALL MOLECULES THAT BIND CYCLIN-DEPENDENT KINASE INHIBITOR 1B (P27KIP1)” having Ser. No. 62/817,924, filed Mar. 13, 2019, the contents of which are incorporated by reference in their entirety.

### STATEMENT REGARDING FEDERALLY SPONSORED RESEARCH OR DEVELOPMENT

This invention was made with government support under grant numbers DC015010 and DC013879 awarded by the National Institutes of Health and N00014-18V-2507 awarded by the Office of Naval Research. The government has certain rights in the invention.

### TECHNICAL FIELD

The present disclosure generally relates to compounds that target intrinsically disordered proteins.

### BACKGROUND

Proteins that exhibit intrinsically disordered regions (IDRs) are prevalent in the human proteome and perform wide ranging functions that control cellular behavior, including signaling and regulation. Intrinsically disordered proteins (IDPs) are associated with a number of human diseases, including cancer, cardiovascular disease, amyloidoses, neurodegenerative diseases, and diabetes. IDPs are challenging targets because they exist as ensembles of structures, which can make standard rational drug design approaches difficult because they require the knowledge of the three-dimensional structure of the proteins to be drugged. Despite knowledge of their numerous disease associations, limited attention has been given to the development of strategies for therapeutically targeting IDPs and proteins with IDRs (Heller, G. T. et al., Cell Mol Life Sci, 2017. 74(17): p. 3225-3243) and they are often considered “undruggable” (Dang, C. V., et al., Nat Rev Cancer, 2017. 17(8): p. 502-508.).

The IDP, p27^{KiP1}, is a regulator of the cyclin-dependent kinases (Cdks) that control cell division in humans (Iconaru, L. I., et al., Sci Rep, 2015. 5: p. 15686). p27 is mislocalized from the nucleus to the cytoplasm in certain cancers, where it interacts with RhoA and alters cell motility (Phillips, A. H., et al., J Mol Biol, 2018. 430(6): p. 751-758). Further, expression of p27 in cells of the inner ear prevents reentry into the cell division cycle that could otherwise enable hearing regeneration in hearing damaged individuals (Walters, B. J., et al., J Neurosci, 2014. 34(47): p. 15751-63.). Small molecules that bind to p27 and inhibit interactions with its Cdk partners in hearing cells and RhoA in cancer cells thus could have therapeutic applications.

There remains a need for improved compounds capable of binding to p27 that overcome the aforementioned deficiencies.

### SUMMARY

In various aspects, compounds and pharmaceutically acceptable salts thereof are provided that overcome one or more of the aforementioned deficiencies. Pharmaceutical formulations containing the compounds or pharmaceutically acceptable salts are also provided along with methods of using the compounds and salts and formulations thereof.

In some aspects, a compound or a pharmaceutically acceptable salt thereof is provided where the compound has a structure according to Formula I, where R¹ is a linear or branched, C₁-C₃ alkyl linker; and where each occurrence of R³⁰ and R³¹ is independently a hydrogen, C₁-C₃ alkyl, or a C₁-C₃ alkoxy.

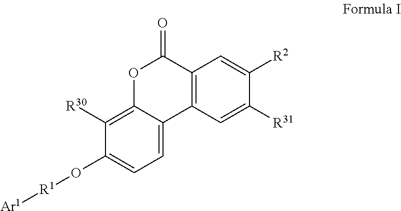

In some aspects, the compound has a structure according to Formula I where R² is a hydrogen, a C₁-C₃ alkyl, or a C₁-C₃ alkoxy; and Ar¹ is selected from the group consisting of

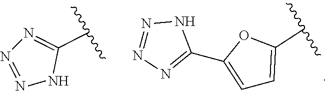 [SMILES: *c1nnn[nH]1 ; *c1ccc(-c2nnn[nH]2)o1]

In some aspects, the compound has a structure according to Formula I where R² is —O—R¹—Ar¹; and each occurrence of Ar¹ is independently selected from the group consisting of

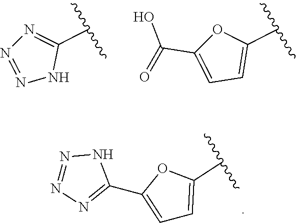 [SMILES: *c1nnn[nH]1 ; *c1ccc(C(=O)O)o1 ; *c1ccc(-c2nnn[nH]2)o1]

In some aspects, a compound or a pharmaceutically acceptable salt thereof is provided where the compound has a structure according to Formula II, where each occurrence of R³⁰ and R³¹ is independently a hydrogen, a halo, a cyano, a hydroxyl, —NH₂, a C₁-C₃ alkyl, a C₁-C₃ haloalkyl, a C₁-C₃ alkoxy, or a C₁-C₃ haloalkoxy; where R² is a hydrogen, a halo, a cyano, a hydroxyl, —NH₂, a C₁-C₃ alkyl, a C₁-C₃ haloalkyl, a C₁-C₃ alkoxy, a C₁-C₃ haloalkoxy, or —O—R¹—Ar²¹—Ar²²; and where each occurrence of R¹ and R⁴ is independently a linear or branched chain, substituted or unsubstituted C₁-C₇ alkyl linker.

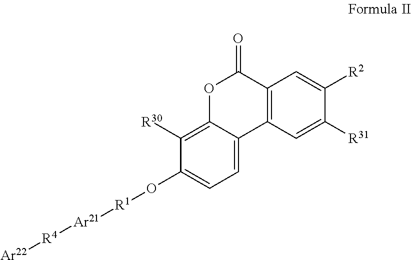

In some aspects, the compound has a structure according to Formula II where each occurrence of Ar²¹ is independently a bond or selected from one of the following structures, where each occurrence of R⁴⁰, R⁴¹, R⁴², and R⁴³ is independently a hydrogen, a halo, a cyano, a hydroxyl, —NH₂, a C₁-C₃ alkyl, a C₁-C₃ haloalkyl, a C₁-C₃ alkoxy, or a C₁-C₃ haloalkoxy.

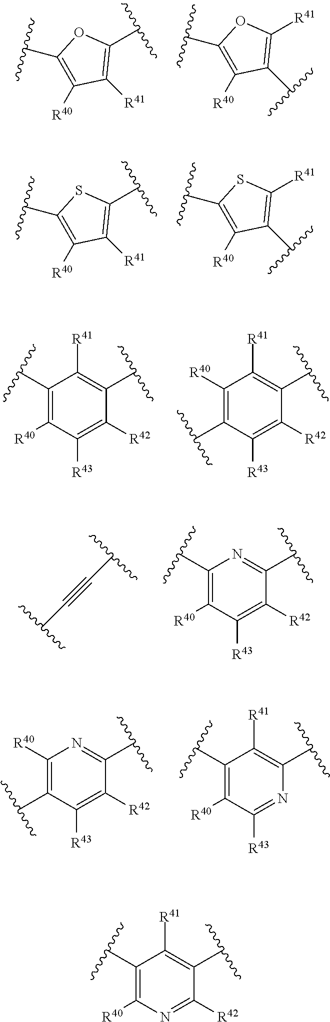

In some aspects, the compound has a structure according to Formula II where each occurrence of Ar²² is independently selected from the following structures, where R⁵ is independently hydrogen, a C₁-C₃ alkyl, or a C₁-C₃ alkoxy.

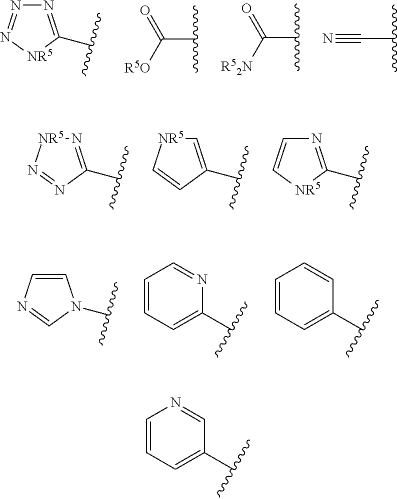

In some aspects, the compound has a structure according to Formula I or Formula II wherein R¹ is —CH₂—. In some aspects, the compound has a structure according to Formula I or Formula II wherein R¹ is —C(CH₃)H—. In some aspects, the compound has a structure according to Formula I or Formula II wherein R¹ is a linear or branched, C₁-C₃ alkyl linker. In some aspects, the compound has a structure according to Formula I or Formula II wherein R⁴ is —CH₂—. In some aspects, the compound has a structure according to Formula I or Formula II wherein R⁴ is —C(CH₃)H—. In some aspects, the compound has a structure according to Formula I or Formula II wherein R⁴ is a linear or branched, C₁-C₃ alkyl linker. In some aspects, the compound has a structure according to Formula I or Formula II wherein R³⁰ is methyl and R³¹ is hydrogen. In some aspects, the compound has a structure according to Formula I or Formula II wherein R³¹ is methyl and R³⁰ is hydrogen. In some aspects, the compound has a structure according to Formula I or Formula II wherein R³⁰ and R³¹ are methyl. In some aspects, the compound has a structure according to Formula I or Formula II wherein R³⁰ and R³¹ are hydrogen. In some aspects, the compound has a structure according to Formula I or Formula II wherein one or both of R³⁰ and R³¹ is hydrogen.

In some aspects, the compound has a structure according to one of the following formulas

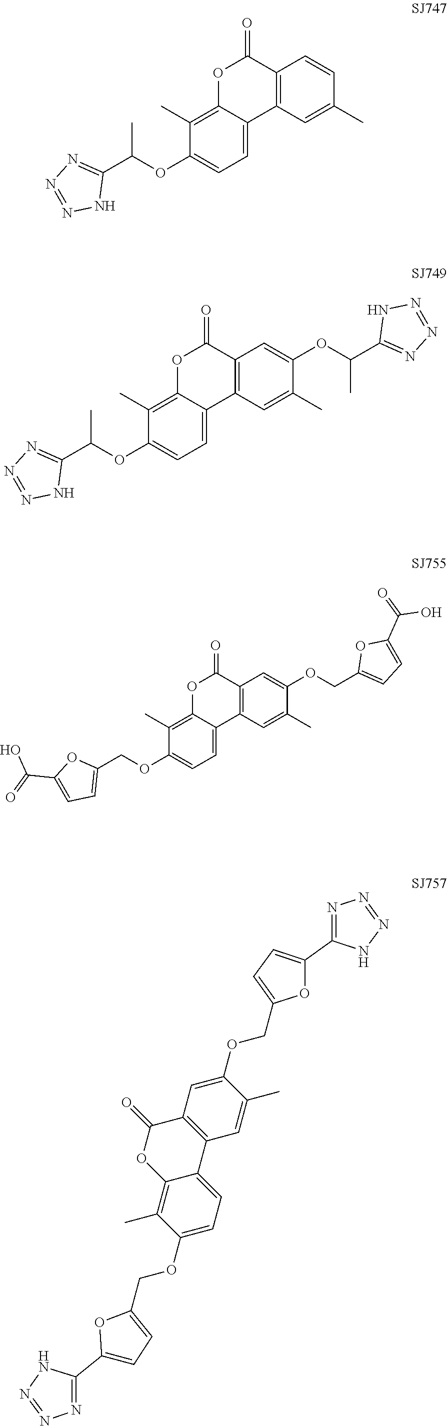 [SMILES: Cc1ccc2c(=O)oc3c(C)c(OC(C)c4nnn[nH]4)ccc3c2c1 ; Cc1cc2c(cc1OC(C)c1nnn[nH]1)c(=O)oc1c(C)c(OC(C)c3nnn[nH]3)ccc12 ; Cc1cc2c(cc1OCc1ccc(C(=O)O)o1)c(=O)oc1c(C)c(OCc3ccc(C(=O)O)o3)ccc12 ; Cc1cc2c(cc1OCc1ccc(-c3nnn[nH]3)o1)c(=O)oc1c(C)c(OCc3ccc(-c4nnn[nH]4)o3)ccc12]

In various aspects, pharmaceutical formulations are provided containing a compound described herein or a pharmaceutically acceptable salt thereof and a pharmaceutically acceptable carrier. The formulations can be a solid dosage form such as a capsule, a tablet, a pill, a powder, a granule, an effervescing granule, a gel, a paste, a troche, or a pastille. The formulations can be a liquid dosage form such as an emulsion, a solution, a suspension, a syrup, or an elixir. In some aspects, the pharmaceutical formulation can include a second active agent and/or can be given in combination with a second active agent. In particular aspects, the second active agent is a cancer therapeutic.

In various aspects, methods for the treatment of a disease or disorder are provided. The methods can include administering a therapeutically effective amount of a compound described herein or a pharmaceutically acceptable salt thereof. The methods can include administering a therapeutically effective amount of a pharmaceutical formulation described herein. In some aspects, the disease or disorder is a cancer. In some aspects, the cancer is associated with a mislocalization of the intrinsically disordered protein p27. In some aspects, the cancer is resistant to an anticancer therapy.

In various aspects, methods of promoting reentry into the cell division cycle in a subject in need thereof are also provided. The methods can include administering a therapeutically effective amount of a compound described herein or a pharmaceutically acceptable salt thereof. The methods can include administering a therapeutically effective amount of a pharmaceutical formulation described herein. In some aspects, the subject has hearing damage or hearing loss and the method includes enabling a regeneration of hearing in the subject.

Other systems, methods, features, and advantages of the compounds described herein, formulations thereof, methods of making thereof, and methods of use thereof will be or become apparent to one with skill in the art upon examination of the following drawings and detailed description. It is intended that all such additional compounds, methods, features, and advantages be included within this description, be within the scope of the present disclosure, and be protected by the accompanying claims.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1A is a schematic of the kinase inhibitory domain of p27 (p27-KID) showing regions D1 (binds to cyclin A), D2 (binds to Cdk2), and linker helix (LH). Also depicted are p27 residues within the subdomain D2 that interact with small molecules. FIG. 1B is a schematic of the Group 1 scaffold optimization showing SAR-by-catalog and SAR-by-synthesis approaches.

FIGS. 2A-2F demonstrate that synthetic compounds generated by growing G1.1 scaffold interact specifically with p27-KID. Chemical shift perturbation (FIG. 2A, FIG. 2C, and FIG. 2E) and peak intensity loss (FIG. 2B, FIG. 2D, and FIG. 2F) histograms obtained by analysis of two-dimensional (2D) ¹H-¹⁵N HSQC NMR spectra of ¹⁵N-p27-KID upon interaction with SJ749 (FIGS. 2A-2B), SJ755 (FIGS. 2C-2D), and SJ757 (FIGS. 2E-2F), respectively.

FIGS. 3A-3G show the results from sedimentation velocity analytical ultracentrifugation (SV-AUC) demonstrating that synthetic compounds induced formation of soluble oligomers of p27-KID. (FIGS. 3A-3C). Sedimentation coefficient distributions c(s) for p27-KID alone (black) and with compounds SJ749 (FIG. 3A), SJ755 (FIG. 3B), and SJ757 (FIG. 3C), respectively. Two-dimensional size-and-shape distribution analyses for the boxed region for p27-KID alone (dashed box region around sedimentation coefficient, S=1 in FIGS. 3A-3C) is shown in FIG. 3D. Two-dimensional size-and-shape distribution analyses for the boxed region for p27-KID with SJ749 from FIG. 3A is shown in FIG. 3E. Two-dimensional size-and-shape distribution analyses for the boxed region for p27-KID with SJ755 from FIG. 3B is shown in FIG. 3F. Two-dimensional size-and-shape distribution analyses for the boxed region for p27-KID with SJ757 from FIG. 3C is shown in FIG. 3G.

FIGS. 4A-4B demonstrate that mutation of W60 and/or W76 to alanine affects binding of p27 to Cdk2/cyclin A. Isothermal titration calorimetry data and binding isotherms for interaction of Cdk2/cyclin A with p27-D2 (FIG. 4A) and p27-KID (FIG. 4B), respectively. The p27-D2 mutants do not bind Cdk2/cyclin A (FIG. 4A), while the p27-KID mutants still bind Cdk2/cyclin A due to interactions between the p27-D1 region with cyclin A (FIG. 4B). These data demonstrate that small molecules that bind to the residues in wild-type p27 that are mutated here may displace the D2 domain (of p27) from Cdk2.

FIG. 5 demonstrates mutation of W60 and W76 in p27-KID to alanine reduced inhibitory potency and prevented full inhibition of Cdk2 catalytic activity toward the substrate, Histone H1. The catalytic activity of Cdk2 within the Cdk2/cyclin A complex at the lowest concentration of p27-KID (black data points) and p27-KID-W60A-W76A (green data points) was normalized to 100%. These data further demonstrate that small molecules that bind to the residues in wild-type p27 that are mutated here may displace the D2 domain (of p27) from Cdk2.

FIGS. 6A-6B show chemical structures of Group 1 (FIG. 6A) and Group 2 (FIG. 6B) compounds used for cheminformatics analysis. FIG. 6C is a schematic for the cheminformatics analysis of compounds in FIG. 6A and FIG. 6B that guided purchase of compounds (termed analog-by-catalog, ABC). FIGS. 6D-6E show results for screening of Group 1 (FIG. 6D) and Group 2 (FIG. 6E) scaffolds showing substituents identified by SAR-by-catalog. Substituents indicated in gray exhibited binding to p27-KID; those in black did not.

FIGS. 7A-7C show interaction of p27-KID with analog-by-catalog compound, ABC-1. FIG. 7A is an overlay of 2D ¹H-¹⁵N HSQC NMR spectra of ¹⁵N-p27-KID alone (100 μM, gray) and with the compound (black). FIGS. 7B-7C show chemical shift perturbation value (FIG. 7B) and relative peak intensity (I/I0) value (FIG. 7C) histograms obtained by analysis of 2D ¹H-¹⁵N HSQC NMR spectra displayed in FIG. 7A. The inset in FIG. 7B shows chemical shift perturbations for sidechain resonances W60, W76, and N66, respectively, from left to right.

FIGS. 8A-8C show interaction of p27-KID with synthesized analog, SJ747. FIG. 8A is an overlay of 2D ¹H-¹⁵N HSQC NMR spectra of ¹⁵N-p27-KID alone (100 μM, gray) and with the compound (black). FIGS. 8B-8C show chemical shift perturbation value (FIG. 8B) and relative peak intensity (I/I0) value (FIG. 8C) histograms obtained by analysis of 2D ¹H-¹⁵N HSQC NMR spectra displayed in FIG. 8A. The inset in FIG. 8B shows chemical shift perturbations for sidechain resonances W60, W76, and N66, respectively, from left to right.

FIGS. 9A-9C show interaction of p27-KID with analog-by-catalog, ABC-2. FIG. 9A is an overlay of 2D ¹H-¹⁵N HSQC NMR spectra of ¹⁵N-p27-KID alone (100 μM, gray) and with the compound (black). FIGS. 9B-9C show chemical shift perturbation value (FIG. 9B) and relative peak intensity (I/I0) value (FIG. 9C) histograms obtained by analysis of 2D ¹H-¹⁵N HSQC NMR spectra displayed in FIG. 9A. The inset in FIG. 9B shows chemical shift perturbations for sidechain resonances W60, W76, and N66, respectively, from left to right.

FIGS. 10A-10C show interaction of p27-KID with synthetic compound, SJ749. FIG. 10A is an overlay of 2D ¹H-¹⁵N HSQC NMR spectra of ¹⁵N-p27-KID in the absence (100 μM, gray) and presence (black) of SJ749, respectively. FIGS. 10B-10C show binding isotherms of select p27 residues that interact with SJ749. Chemical shift perturbation (FIG. 10B) and relative peak intensity (I/I0) values (%) (FIG. 10C) are plotted versus compound concentration.

FIG. 11 is a plot of the 1D ¹H (black) and water LOGSY (gray) NMR spectra of SJ749 (upper curves); SJ755 (middle curves); and SJ757 (lower curves), respectively. Negative peaks in the WaterLOGSY spectra indicate the compounds do not bind to the p27 protein in aqueous solutions.

FIGS. 12A-12C show interaction of p27-KID with synthetic compound, SJ755. FIG. 12A is an overlay of 2D ¹H-¹⁵N HSQC NMR spectra of ¹⁵N-p27-KID in the absence (100 μM, gray) and presence (black) of SJ755, respectively. FIGS. 12B-12C show binding isotherms of select p27 residues that interact with SJ755. Chemical shift perturbation (FIG. 12B) and relative peak intensity (I/I0) values (%) (FIG. 12C) are plotted versus compound concentration.

FIGS. 13A-13C show interaction of p27-KID with synthetic compound, SJ757. FIG. 13A is an overlay of 2D ¹H-¹⁵N HSQC NMR spectra of ¹⁵N-p27-KID in the absence (25 μM, gray) and presence (black) of SJ757, respectively. FIGS. 13B-13C show binding isotherms of select p27 residues that interact with SJ757. Chemical shift perturbation (FIG. 13B) and relative peak intensity (I/I0) values (%) (FIG. 13C) are plotted against compound concentration.

FIGS. 14A-14C show mutation of tryptophan residues within p27-KID drastically reduces interaction of p27-KID-W60A-W76A with synthetic compound SJ749. FIG. 14A is an overlay of 2D ¹H-¹⁵N HSQC NMR spectra of ¹⁵N-p27-KID-W60A-W76A alone (100 μM, gray) and with the SJ749 (black). FIGS. 14B-14C show chemical shift perturbation (FIG. 14B) and relative peak intensity (I/I0) value (%) (FIG. 14C) histograms obtained by analysis of 2D ¹H-¹⁵N HSQC NMR spectra displayed in FIG. 14A.

FIGS. 15A-15C show mutation of tryptophan residues within p27-KID drastically reduces interaction of p27-KID-W60A-W76A with synthetic compound SJ755. FIG. 15A is an overlay of 2D ¹H-¹⁵N HSQC NMR spectra of ¹⁵N-p27-KID-W60A-W76A alone (100 μM, gray) and with the SJ755 (black). FIGS. 15B-15C show chemical shift perturbation (FIG. 15B) and relative peak intensity (I/I0) value (%) (FIG. 15C) histograms obtained by analysis of 2D ¹H-¹⁵N HSQC NMR spectra displayed in FIG. 15A.

FIGS. 16A-16F demonstrate that tryptophan residues within p27-KID contribute to the interaction with compound SJ757. FIG. 16A is an overlay of 2D ¹H-¹⁵N HSQC NMR spectra of ¹⁵N-p27-KID-W60A-W76A in the absence (25 μM, light gray) and presence of SJ757 (dark gray, ratio 1:2; black, ratio 1:4). Selected residues show chemical shift perturbations and peak intensity loss. FIGS. 16B-16C show chemical shift perturbation (FIG. 16B) and relative peak intensity (I/I0) value (%) (FIG. 16C) histograms obtained by analysis of 2D ¹H-¹⁵N HSQC NMR spectra displayed in FIG. 16A. FIGS. 16D-16F show analytical ultracentrifugation (AUC) results reveal that SJ757 causes formation of soluble oligomers upon binding to p27-KID-W60A-W76A. FIG. 16D is a plot of sedimentation coefficient distributions c(s) of p27-KID-W60A-W76A alone (black) and with compound SJ757 (gray). FIGS. 16E-16F are two-dimensional size-and-shape distribution analyses of the sedimentation velocity data presented in FIG. 16D: p27-KID-W60A-W76A alone (FIG. 16E) and with compound SJ757 (FIG. 16F). Iso-S lines (for which the S value is the same) are labeled with the corresponding sedimentation coefficient values.

FIGS. 17A-17B demonstrate that mutation of W60 and/or W76 to alanine affects binding of p27 to Cdk2. The figure shows isothermal titration calorimetry data and binding isotherms for interaction of Cdk2 with p27-KID (FIG. 17A) and p27-D2 (FIG. 17B) variants, respectively.

FIGS. 18A-180 and FIGS. 18F-18I show results of Cdk2 phosphorylation activity assays for Cdk2/cyclin A (100 μM) in the presence of increasing concentrations of p27 variants: p27-KID (FIG. 18A), p27-KIDW60A-W76A (FIG. 18B), p27-KID-W60A (FIG. 18C), p27-KID-W76A (FIG. 18D), p27-D2 (FIG. 18F), p27-D2-W60A-W76A (FIG. 18G), p27-D2-W60A (FIG. 18H), and p27-D2-W76A (FIG. 18I). The panels show phosphoimager results after SDS-PAGE analysis of ³²P incorporation from ATP into the substrate, Histone H1. A single set of representative results are shown; all experiments were performed in triplicate. FIG. 18E and FIG. 18J show kinase inhibition curves for p27-KID (FIG. 18E) and p27-D2 (FIG. 18J) variants.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only and is not intended to be limiting. The skilled artisan will recognize many variants and adaptations of the embodiments described herein. These variants and adaptations are intended to be included in the teachings of this disclosure and to be encompassed by the claims herein.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant specification should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

Although any methods and materials similar or equivalent to those described herein can also be used in the practice or testing of the present disclosure, the preferred methods and materials are now described. Functions or constructions well-known in the art may not be described in detail for brevity and/or clarity. Embodiments of the present disclosure will employ, unless otherwise indicated, techniques of biotechnology, molecular biology, microbiology, medicinal chemistry, organic chemistry, biochemistry, physiology, cell biology, physiology, medicine, and the like, which are within the skill of the art. Such techniques are explained fully in the literature.

### Definitions

Unless defined otherwise, all technical and scientific terms used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within ±10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and juvenile subjects, whether male or female, are intended to be covered. In one aspect, the subject is a mammal. A patient refers to a subject afflicted with a disease or disorder. The term “patient” includes human and veterinary subjects.

As used herein, the term “diagnosed” means having been subjected to a physical examination by a person of skill, for example, a physician, and found to have a condition that can be diagnosed or treated by the compounds, compositions, or methods disclosed herein.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, chickens, turkeys, etc.), and laboratory animals (e.g., mouse, rabbit, rat, guinea pig, fruit fly, etc.).

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravitreal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The terms “sufficient” and “effective”, as used interchangeably herein, refer to an amount (e.g., mass, volume, dosage, concentration, and/or time period) needed to achieve one or more desired result(s). A “therapeutically effective amount” is at least the minimum concentration required to effect a measurable improvement or prevention of any symptom or a particular condition or disorder, to effect a measurable enhancement of life expectancy, or to generally improve patient quality of life. The therapeutically effective amount is thus dependent upon the specific biologically active molecule and the specific condition or disorder to be treated. Therapeutically effective amounts of many active agents, such as antibodies, are well known in the art. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

The term “prodrug” refers to an agent, including a nucleic acid or proteins that is converted into a biologically active form in vitro and/or in vivo. Prodrugs can be useful because, in some situations, they may be easier to administer than the parent compound. For example, a prodrug may be bioavailable by oral administration whereas the parent compound is not. The prodrug may also have improved solubility in pharmaceutical compositions compared to the parent drug. A prodrug may be converted into the parent drug by various mechanisms, including enzymatic processes and metabolic hydrolysis. Harper, N.J. (1962) Drug Latentiation in Jucker, ed. Progress in Drug Research, 4:221-294; Morozowich et al. (1977) Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977) Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997) Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11:345-365; Gaignault et al. (1996) Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogues, Adv. Drug Delivery Rev., 39(1-3):183-209; Browne (1997). Fosphenytoin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996) Improved oral drug delivery: solubility limitations overcome by the use of prodrugs, Adv. Drug Delivery Rev. 19(2): 115-130; Fleisher et al. (1985) Design of prodrugs for improved gastrointestinal absorption by intestinal enzyme targeting, Methods Enzymol. 112: 360-81; Farquhar D, et al. (1983) Biologically Reversible Phosphate-Protective Groups, J. Pharm. Sci., 72(3): 324-325; Han, H. K. et al. (2000) Targeted prodrug design to optimize drug delivery, AAPS PharmSci., 2(1): E6; Sadzuka Y. (2000) Effective prodrug liposome and conversion to active metabolite, Curr. Drug Metab., 1(1):31-48; D. M. Lambert (2000) Rationale and applications of lipids as prodrug carriers, Eur. J. Pharm. Sci., 11 Suppl. 2:S15-27; Wang, W. et al. (1999) Prodrug approaches to the improved delivery of peptide drugs. Curr. Pharm. Des., 5(4):265-87.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, “dosage form” means a pharmacologically active material in a medium, carrier, vehicle, or device suitable for administration to a subject. A suitable dosage form can comprise a compound according to Formula I, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium deoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound according to Formula I, a product of a disclosed method of making, or a salt, solvate, or polymorph thereof, suspended in sterile saline solution for injection together with a preservative.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, π-π interactions, cation-π interactions, anion-π interactions, polar π-interactions, and hydrophobic effects.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

As used herein, “therapeutic” can refer to treating, healing, and/or ameliorating a disease, disorder, condition, or side effect, or to decreasing in the rate of advancement of a disease, disorder, condition, or side effect.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

As used herein, the term “pharmaceutically acceptable carrier” refers to aqueous or nonaqueous solutions, dispersions, suspensions or emulsions, as well as powders for reconstitution into injectable solutions or dispersions just prior to use. Preferably, a pharmaceutically acceptable carrier will be sterile or sterilizable, e.g., where the pharmaceutical composition is intended for injection. The pharmaceutically acceptable carrier is advantageously selected so as not to significantly decrease or neutralize the active ingredient. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microcapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMBIODRAW™ (Cambridgesoft Corporation, U.S.A.). Compounds were generally named herein using CHEMBIODRAW™ (v. 14.0.0.117).

The term “small molecule”, as used herein, generally refers to an organic molecule that is less than 2000 g/mol in molecular weight, less than 1500 g/mol, less than 1000 g/mol, less than 800 g/mol, or less than 500 g/mol. Small molecules are non-polymeric and/or non-oligomeric.

The term “hydrophilic”, as used herein, refers to substances that have strongly polar groups that readily interact with water.

The term “hydrophobic”, as used herein, refers to substances that lack an affinity for water; tending to repel and not absorb water as well as not dissolve in or mix with water.

The term “lipophilic”, as used herein, refers to compounds having an affinity for lipids.

The term “amphiphilic”, as used herein, refers to a molecule combining hydrophilic and lipophilic (hydrophobic) properties. “Amphiphilic material” as used herein refers to a material containing a hydrophobic or more hydrophobic oligomer or polymer (e.g., biodegradable oligomer or polymer) and a hydrophilic or more hydrophilic oligomer or polymer.

The term “targeting moiety”, as used herein, refers to a moiety that binds to or localizes to a specific locale. The moiety may be, for example, a protein, nucleic acid, nucleic acid analog, carbohydrate, or small molecule. The locale may be a tissue, a particular cell type, or a subcellular compartment. In some embodiments, a targeting moiety can specifically bind to a selected molecule.

The term “reactive coupling group”, as used herein, refers to any chemical functional group capable of reacting with a second functional group to form a covalent bond. The selection of reactive coupling groups is within the ability of the skilled artisan. Examples of reactive coupling groups can include primary amines (—NH₂) and amine-reactive linking groups such as isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, and fluorophenyl esters. Most of these conjugate to amines by either acylation or alkylation. Examples of reactive coupling groups can include aldehydes (—COH) and aldehyde reactive linking groups such as hydrazides, alkoxyamines, and primary amines. Examples of reactive coupling groups can include thiol groups (—SH) and sulfhydryl reactive groups such as maleimides, haloacetyls, and pyridyl disulfides. Examples of reactive coupling groups can include photoreactive coupling groups such as aryl azides or diazirines. The coupling reaction may include the use of a catalyst, heat, pH buffers, light, or a combination thereof.

The term “protective group”, as used herein, refers to a functional group that can be added to and/or substituted for another desired functional group to protect the desired functional group from certain reaction conditions and selectively removed and/or replaced to deprotect or expose the desired functional group. Protective groups are known to the skilled artisan. Suitable protective groups may include those described in Greene, T. W. and Wuts, P. G. M., Protective Groups in Organic Synthesis, (1991). Acid sensitive protective groups include dimethoxytrityl (DMT), tert-butylcarbamate (tBoc) and trifluoroacetyl (tFA). Base sensitive protective groups include 9-fluorenylmethoxycarbonyl (Fmoc), isobutyrl (iBu), benzoyl (Bz) and phenoxyacetyl (pac). Other protective groups include acetamidomethyl, acetyl, tert-amyloxycarbonyl, benzyl, benzyloxycarbonyl, 2-(4-biphenylyl)-2-propyloxycarbonyl, 2-bromobenzyloxycarbonyl, tert-butyl₇ tert-butyloxycarbonyl, I-carbobenzoxamido-2,2,2-trifluoroethyl, 2,6-dichlorobenzyl, 2-(3,5-dimethoxyphenyl)-2-propyloxycarbonyl, 2,4-dinitrophenyl, dithiasuccinyl, formyl, 4-methoxybenzenesulfonyl, 4-methoxybenzyl, 4-methylbenzyl, o-nitrophenylsulfenyl, 2-phenyl-2-propyloxycarbonyl, α-2,4,5-tetramethylbenzyloxycarbonyl, p-toluenesulfonyl, xanthenyl, benzyl ester, N-hydroxysuccinimide ester, p-nitrobenzyl ester, p-nitrophenyl ester, phenyl ester, p-nitrocarbonate, p-nitrobenzylcarbonate, trimethylsilyl and pentachlorophenyl ester.

The term “activated ester”, as used herein, refers to alkyl esters of carboxylic acids where the alkyl is a good leaving group rendering the carbonyl susceptible to nucleophilic attack by molecules bearing amino groups. Activated esters are therefore susceptible to aminolysis and react with amines to form amides. Activated esters contain a carboxylic acid ester group —CO₂R where R is the leaving group.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups.

In some embodiments, a straight chain or branched chain alkyl has 30 or fewer carbon atoms in its backbone (e.g., C₁-C₃₀ for straight chains, C₃-C₃₀ for branched chains), 20 or fewer, 12 or fewer, or 7 or fewer. Likewise, in some embodiments cycloalkyls have from 3-10 carbon atoms in their ring structure, e.g. have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a hosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons, or from one to six carbon atoms in its backbone structure. Likewise, “lower alkenyl” and “lower alkynyl” have similar chain lengths. Throughout the application, preferred alkyl groups are lower alkyls. In some embodiments, a substituent designated herein as alkyl is a lower alkyl.

It will be understood by those skilled in the art that the moieties substituted on the hydrocarbon chain can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

The term “alkylthio” refers to an alkyl group, as defined above, having a sulfur radical attached thereto. In some embodiments, the “alkylthio” moiety is represented by one of —S— alkyl, —S-alkenyl, and —S-alkynyl. Representative alkylthio groups include methylthio, and ethylthio. The term “alkylthio” also encompasses cycloalkyl groups, alkene and cycloalkene groups, and alkyne groups. “Arylthio” refers to aryl or heteroaryl groups. Alkylthio groups can be substituted as defined above for alkyl groups.

The terms “alkenyl” and “alkynyl”, refer to unsaturated aliphatic groups analogous in length and possible substitution to the alkyls described above, but that contain at least one double or triple bond respectively.

The terms “alkoxyl” or “alkoxy” as used herein refers to an alkyl group, as defined above, having an oxygen radical attached thereto. Representative alkoxyl groups include methoxy, ethoxy, propyloxy, and tert-butoxy. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O— alkynyl. Aroxy can be represented by —O-aryl or O-heteroaryl, wherein aryl and heteroaryl are as defined below. The alkoxy and aroxy groups can be substituted as described above for alkyl.

The terms “amine” and “amino” are art-recognized and refer to both unsubstituted and substituted amines, e.g., a moiety that can be represented by the general formula:

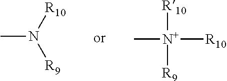

wherein R₉, R₁₀, and R′₁₀ each independently represent a hydrogen, an alkyl, an alkenyl, —(CH₂)ₘ—R₈ or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In some embodiments, only one of R₉ or R₁₀ can be a carbonyl, e.g., R₉, R₁₀ and the nitrogen together do not form an imide. In still other embodiments, the term “amine” does not encompass amides, e.g., wherein one of R₉ and R₁₀ represents a carbonyl. In additional embodiments, R₉ and R₁₀ (and optionally R′₁₀) each independently represent a hydrogen, an alkyl or cycloakly, an alkenyl or cycloalkenyl, or alkynyl. Thus, the term “alkylamine” as used herein means an amine group, as defined above, having a substituted (as described above for alkyl) or unsubstituted alkyl attached thereto, i.e., at least one of R₉ and R₁₀ is an alkyl group.

The term “amido” is art-recognized as an amino-substituted carbonyl and includes a moiety that can be represented by the general formula:

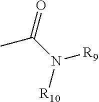

wherein R₉ and R₁₀ are as defined above.

“Aryl”, as used herein, refers to C₅-C₁₀-membered aromatic, heterocyclic, fused aromatic, fused heterocyclic, biaromatic, or bihetereocyclic ring systems. Broadly defined, “aryl”, as used herein, includes 5-, 6-, 7-, 8-, 9-, and 10-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

The term “aralkyl”, as used herein, refers to an alkyl group substituted with an aryl group (e.g., an aromatic or heteroaromatic group).

The term “carbocycle”, as used herein, refers to an aromatic or non-aromatic ring in which each atom of the ring is carbon.

“Heterocycle” or “heterocyclic”, as used herein, refers to a cyclic radical attached via a ring carbon or nitrogen of a monocyclic or bicyclic ring containing 3-10 ring atoms, and preferably from 5-6 ring atoms, consisting of carbon and one to four heteroatoms each selected from the group consisting of non-peroxide oxygen, sulfur, and N(Y) wherein Y is absent or is H, O, (C₁-C₁₀) alkyl, phenyl or benzyl, and optionally containing 1-3 double bonds and optionally substituted with one or more substituents. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Heterocyclic groups can optionally be substituted with one or more substituents at one or more positions as defined above for alkyl and aryl, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, and —CN.

The term “carbonyl” is art-recognized and includes such moieties as can be represented by the general formula:

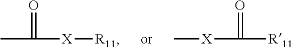

wherein X is a bond or represents an oxygen or a sulfur, and R₁₁ represents a hydrogen, an alkyl, a cycloalkyl, an alkenyl, a cycloalkenyl, or an alkynyl, R′₁₁ represents a hydrogen, an alkyl, a cycloalkyl, an alkenyl, a cycloalkenyl, or an alkynyl. Where X is an oxygen and R₁₁ or R′₁₁ is not hydrogen, the formula represents an “ester”. Where X is an oxygen and R₁₁ is as defined above, the moiety is referred to herein as a carboxyl group, and particularly when R₁₁ is a hydrogen, the formula represents a “carboxylic acid”. Where X is an oxygen and R′₁₁ is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocarboxylic acid.” Where X is a sulfur and R′₁₁ is hydrogen, the formula represents a “thioformate.” On the other hand, where X is a bond, and R₁₁ is not hydrogen, the above formula represents a “ketone” group. Where X is a bond, and R₁₁ is hydrogen, the above formula represents an “aldehyde” group.

The term “monoester” as used herein refers to an analogue of a dicarboxylic acid wherein one of the carboxylic acids is functionalized as an ester and the other carboxylic acid is a free carboxylic acid or salt of a carboxylic acid. Examples of monoesters include, but are not limited to, monoesters of succinic acid, glutaric acid, adipic acid, suberic acid, sebacic acid, azelaic acid, oxalic and maleic acid.

The term “heteroatom” as used herein means an atom of any element other than carbon or hydrogen. Examples of heteroatoms are boron, nitrogen, oxygen, phosphorus, sulfur and selenium. Other heteroatoms include silicon and arsenic.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The term “substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, and polypeptide groups.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. The heteroatoms such as nitrogen may have hydrogen substituents and/or any permissible substituents of organic compounds described herein which satisfy the valences of the heteroatoms.

In various embodiments, the substituent is selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone, each of which optionally is substituted with one or more suitable substituents. In some embodiments, the substituent is selected from alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone, wherein each of the alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone can be further substituted with one or more suitable substituents.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, thioketone, ester, heterocyclyl, —CN, aryl, aryloxy, perhaloalkoxy, aralkoxy, heteroaryl, heteroaryloxy, heteroarylalkyl, heteroaralkoxy, azido, alkylthio, oxo, acylalkyl, carboxy esters, carboxamido, acyloxy, aminoalkyl, alkylaminoaryl, alkylaryl, alkylaminoalkyl, alkoxyaryl, arylamino, aralkylamino, alkylsulfonyl, carboxamidoalkylaryl, carboxamidoaryl, hydroxyalkyl, haloalkyl, alkylaminoalkylcarboxy, aminocarboxamidoalkyl, cyano, alkoxyalkyl, perhaloalkyl, arylalkyloxyalkyl, and the like. In some embodiments, the substituent is selected from cyano, halogen, hydroxyl, and nitro.

The terms “polypeptide,” “peptide” and “protein” generally refer to a polymer of amino acid residues. As used herein, the term also applies to amino acid polymers in which one or more amino acids are chemical analogues or modified derivatives of corresponding naturally-occurring amino acids. The term “protein”, as generally used herein, refers to a polymer of amino acids linked to each other by peptide bonds to form a polypeptide for which the chain length is sufficient to produce tertiary and/or quaternary structure. The term “protein” excludes small peptides by definition, the small peptides lacking the requisite higher-order structure necessary to be considered a protein.

The terms “nucleic acid,” “polynucleotide,” and “oligonucleotide” are used interchangeably to refer to a deoxyribonucleotide or ribonucleotide polymer, in linear or circular conformation, and in either single- or double-stranded form. These terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, and unless otherwise specified, an analogue of a particular nucleotide has the same base-pairing specificity; i.e., an analogue of A will base-pair with T. The term “nucleic acid” is a term of art that refers to a string of at least two base-sugar-phosphate monomeric units. Nucleotides are the monomeric units of nucleic acid polymers. The term includes deoxyribonucleic acid (DNA) and ribonucleic acid (RNA) in the form of a messenger RNA, antisense, plasmid DNA, parts of a plasmid DNA or genetic material derived from a virus. Antisense is a polynucleotide that interferes with the function of DNA and/or RNA. The term “nucleic acids” refers to a string of at least two base-sugar-phosphate combinations. Natural nucleic acids have a phosphate backbone, artificial nucleic acids may contain other types of backbones, but contain the same bases. The term also includes PNAs (peptide nucleic acids), phosphorothioates, and other variants of the phosphate backbone of native nucleic acids.

A “functional fragment” of a protein, polypeptide or nucleic acid is a protein, polypeptide or nucleic acid whose sequence is not identical to the full-length protein, polypeptide or nucleic acid, yet retains at least one function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA binding function of a polypeptide can be determined, for example, by filter-binding, electrophoretic mobility shift, or immunoprecipitation assays. DNA cleavage can be assayed by gel electrophoresis. The ability of a protein to interact with another protein can be determined, for example, by co-immunoprecipitation, two-hybrid assays or complementation, e.g., genetic or biochemical. See, for example, Fields et al. (1989) Nature 340:245-246; U.S. Pat. No. 5,585,245 and PCT WO 98/44350.

As used herein, the term “linker” refers to a carbon chain that can contain heteroatoms (e.g., nitrogen, oxygen, sulfur, etc.) and which may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 atoms long. Linkers may be substituted with various substituents including, but not limited to, hydrogen atoms, alkyl, alkenyl, alkynl, amino, alkylamino, dialkylamino, trialkylamino, hydroxyl, alkoxy, halogen, aryl, heterocyclic, aromatic heterocyclic, cyano, amide, carbamoyl, carboxylic acid, ester, thioether, alkylthioether, thiol, and ureido groups. Those of skill in the art will recognize that each of these groups may in turn be substituted. Examples of linkers include, but are not limited to, pH-sensitive linkers, protease cleavable peptide linkers, nuclease sensitive nucleic acid linkers, lipase sensitive lipid linkers, glycosidase sensitive carbohydrate linkers, hypoxia sensitive linkers, photo-cleavable linkers, heat-labile linkers, enzyme cleavable linkers (e.g., esterase cleavable linker), ultrasound-sensitive linkers, and x-ray cleavable linkers.

The term “pharmaceutically acceptable counter ion” refers to a pharmaceutically acceptable anion or cation. In various embodiments, the pharmaceutically acceptable counter ion is a pharmaceutically acceptable ion. For example, the pharmaceutically acceptable counter ion is selected from citrate, malate, acetate, oxalate, chloride, bromide, iodide, nitrate, sulfate, bisulfate, phosphate, acid phosphate, isonicotinate, acetate, lactate, salicylate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)). In some embodiments, the pharmaceutically acceptable counter ion is selected from chloride, bromide, iodide, nitrate, sulfate, bisulfate, phosphate, acid phosphate, citrate, malate, acetate, oxalate, acetate, and lactate. In particular embodiments, the pharmaceutically acceptable counter ion is selected from chloride, bromide, iodide, nitrate, sulfate, bisulfate, and phosphate.

The term “pharmaceutically acceptable salt(s)” refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, malate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

If the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

A pharmaceutically acceptable salt can be derived from an acid selected from 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, hydrochloric acid, isethionic, isobutyric acid, lactic acid, lactobionic acid, lauric acid, maleic acid, malic acid, malonic acid, mandelic acid, methanesulfonic acid, mucic, naphthalene-1,5-disulfonic acid, naphthalene-2-sulfonic acid, nicotinic acid, nitric acid, oleic acid, oxalic acid, palmitic acid, pamoic acid, pantothenic, phosphoric acid, proprionic acid, pyroglutamic acid, salicylic acid, sebacic acid, stearic acid, succinic acid, sulfuric acid, tartaric acid, thiocyanic acid, toluenesulfonic acid, trifluoroacetic, and undecylenic acid.

The term “bioavailable” is art-recognized and refers to a form of the subject invention that allows for it, or a portion of the amount administered, to be absorbed by, incorporated to, or otherwise physiologically available to a subject or patient to whom it is administered.

Small Molecules that Bind p27Kip1

In a variety of aspects, this disclosure provides small molecules capable of binding to p27Kip1. In some aspects, a compound or a pharmaceutically acceptable salt is provided, the compound having a structure according to Formula I:

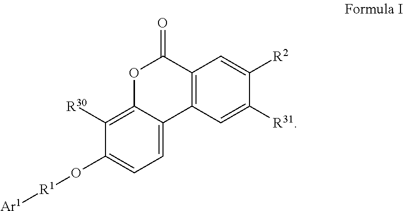

In Formula I, R¹ can be a linear or branched linker, which can be substituted or unsubstituted with one or more substituents such as a linear or branched C₁-C₇, C₁-C₅, or C₁-C₃ alkyl linker. Each occurrence of R³⁰ and R³¹ can independently be a hydrogen, alkyl, or alkoxy, e.g. a substituted or unsubstituted C₁-C₇, C₁-C₅, or C₁-C₃ alkyl or alkoxy.

In some aspects, the compound has a structure according to Formula I wherein R² is a hydrogen, a C₁-C₇ alkyl, a C₁-C₅ alkyl, a C₁-C₃ alkyl, a C₁-C₇ alkoxy, a C₁-C₅ alkoxy. or a C₁-C₃ alkoxy; and Ar¹ is selected from the following structures.

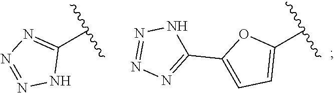 [SMILES: *c1nnn[nH]1 ; *c1ccc(-c2nnn[nH]2)o1]

In some aspects, the compound has a structure according to Formula I wherein R² is —O—R¹—Ar¹; and each occurrence of Ar¹ is independently selected from the following structures.

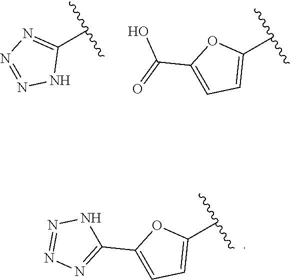 [SMILES: *c1nnn[nH]1 ; *c1ccc(C(=O)O)o1 ; *c1ccc(-c2nnn[nH]2)o1]

In some aspects, a compound or a pharmaceutically acceptable salt is provided, the compound having a structure according to Formula II

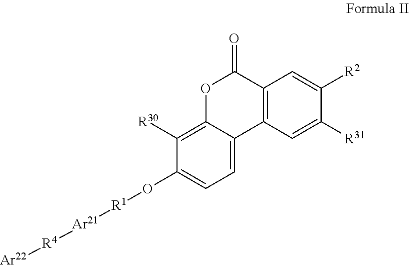

In Formula II, each occurrence of R³⁰ and R³¹ can independently be a hydrogen, a halo, a cyano, a hydroxyl, —NH₂, or a C₁-C₁₂, C₁-C₇, C₁-C₅, or C₁-C₃ alkyl, haloalkyl, alkoxy, or haloalkoxy. In Formula II, R² can be a hydrogen, a halo, a cyano, a hydroxyl, —NH₂, or a C₁-C₁₂, C₁-C₇, C₁-C₅, or C₁-C₃ alkyl, haloalkyl, alkoxy, or haloalkoxy, or R² can be —O—R¹—Ar²¹—Ar²². In Formula II, each occurrence of R¹ and R⁴ can independently be a linear or branched chain, substituted or unsubstituted C₁-C₁₅, C₁-C₁₂, C₁-C₇, C₁-C₅, or C₁-C₃ alkyl linker.

In Formula II, each occurrence of Ar²¹ can independently be a bond or selected from the following structures, where each occurrence of R⁴⁰, R⁴¹, R⁴², and R⁴³ is independently a hydrogen, a halo, a cyano, a hydroxyl, —NH₂, or a C₁-C₁₂, C₁-C₇, C₁-C₅, or C₁-C₃ alkyl, haloalkyl, alkoxy, or haloalkoxy.

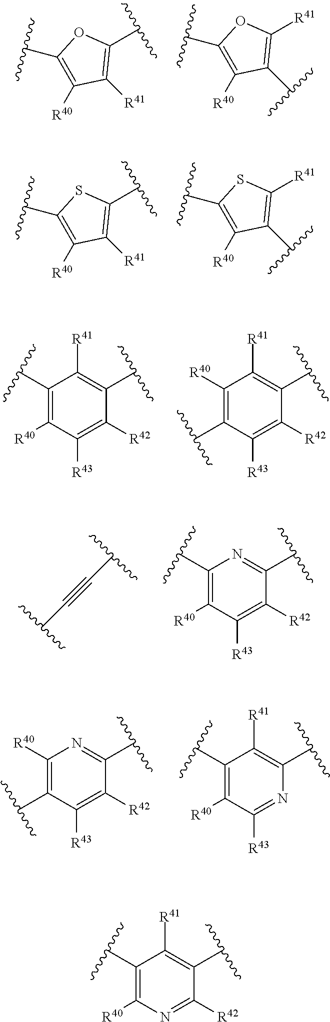

In Formula II, each occurrence of Ar²² can independently be selected from the following structures, where each occurrence of R⁵ is independently hydrogen, or a C₁-C₁₂, C₁-C₇, C₁-C₅, or C₁-C₃ alkyl, haloalkyl, alkoxy, or haloalkoxy.

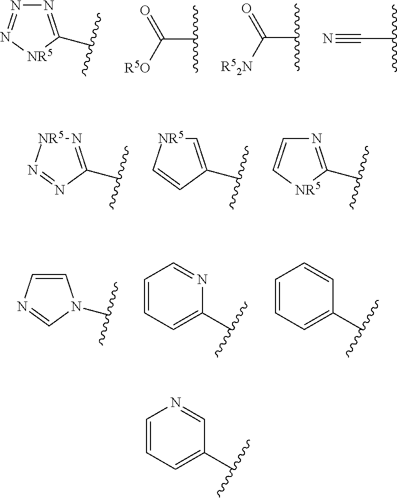

### Methods of Making Compounds

The compounds and salts thereof can be made via a number of synthetic approaches as will become apparent to those skilled in the art. The compounds of this disclosure may be manufactured by the methods provided below, by the methods provided in the examples or by analogous methods. Appropriate reaction conditions for the individual reaction steps are known to a person skilled in the art. Starting materials are either commercially available or can be prepared by methods analogous to the methods given below, by methods described in references cited in the text or in the examples, or by methods known in the art. It is understood that reference to a product of a disclosed method of making a compound is inclusive of the disclosed product, as well as pharmaceutically acceptable salt, hydrate, solvate, or polymorph forms thereof.

The compounds of this invention can be prepared by employing reactions as shown in the disclosed schemes, in addition to other standard manipulations that are known in the literature, exemplified in the experimental sections or clear to one skilled in the art. For clarity, examples having a fewer substituent can be shown where multiple substituents are allowed under the definitions disclosed herein. Thus, the following examples are provided so that the invention might be more fully understood, are illustrative only, and should not be construed as limiting.

In some aspects, a nitrile containing aromatic can be used as the functional handle and diversify it into other compounds described herein via known techniques. In some aspects, the building blocks can be purchased commercially.

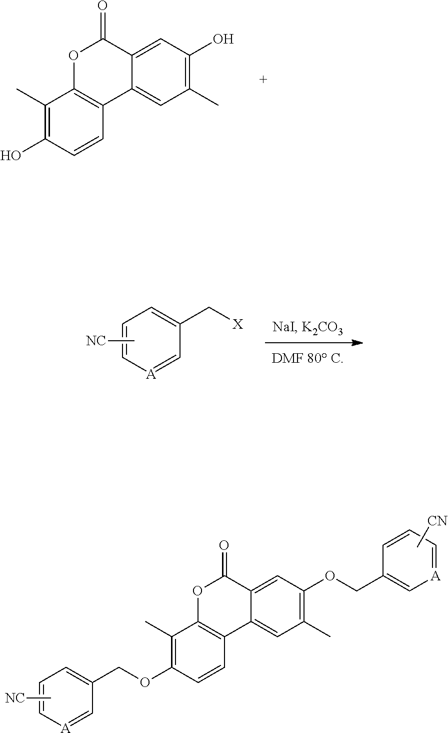

In some aspects, the aromatic can be a commercially available nitrile containing five membered aromatics:

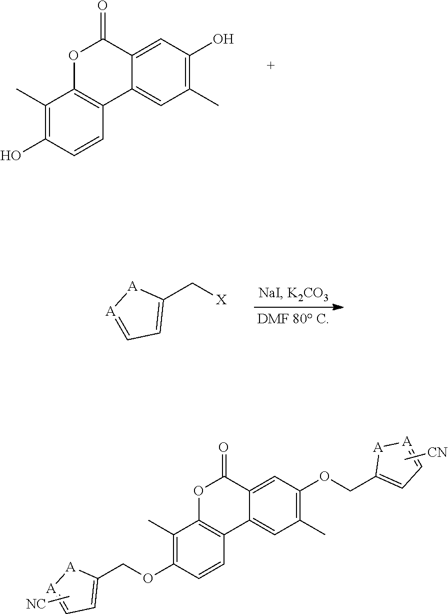

In instances where the aromatic building blocks are not available commercially, commercial cyano-heterocycles, such as oxazole and thiazole can been prepared via radical bromination or bromination of the corresponding alcohol:

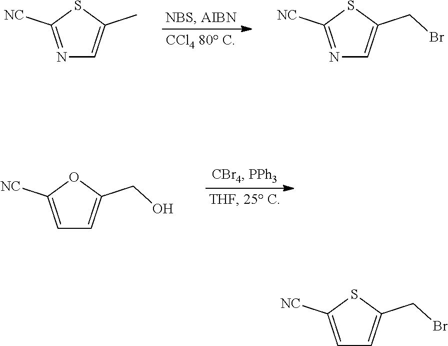

In some aspects, the nitriles can be hydrolyzed to the amide using Cu(OAc)₂ with N,N-diethylhydroxylamine:

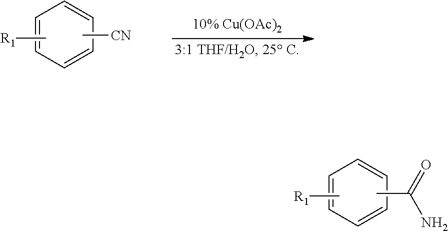

In some instances, hydrolysis of a nitrile to the carboxylic acid can be accomplished under acidic or basic conditions:

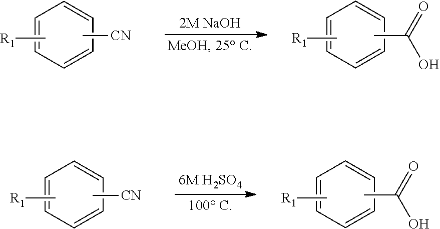

Tetrazoles can be formed using the validated protocol from the carboxylic acid. Alkylated tetrazoles can be formed via amidation of the carboxylic acid followed by chlorination of the amide, followed by treatment with an azide source.

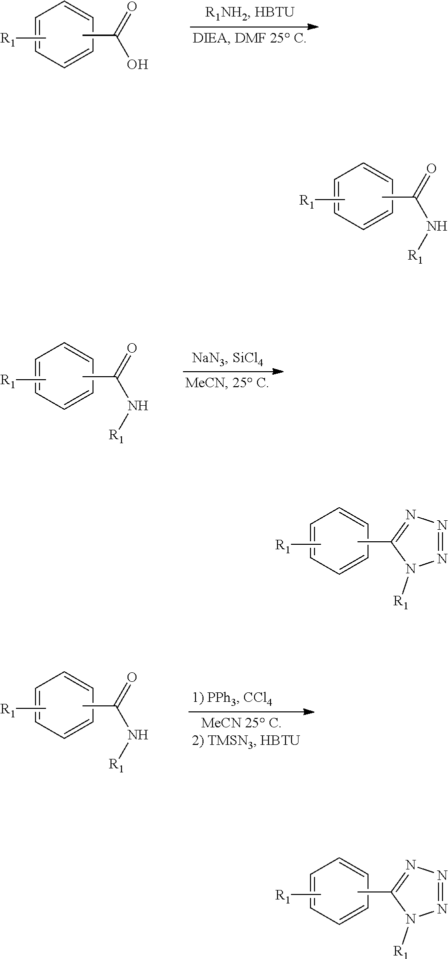

Carbon connected imidazoles can be formed via an extended synthetic procedure from the acid via reduction, ring formation to the dihydroimidazole, followed by imidazole oxidation:

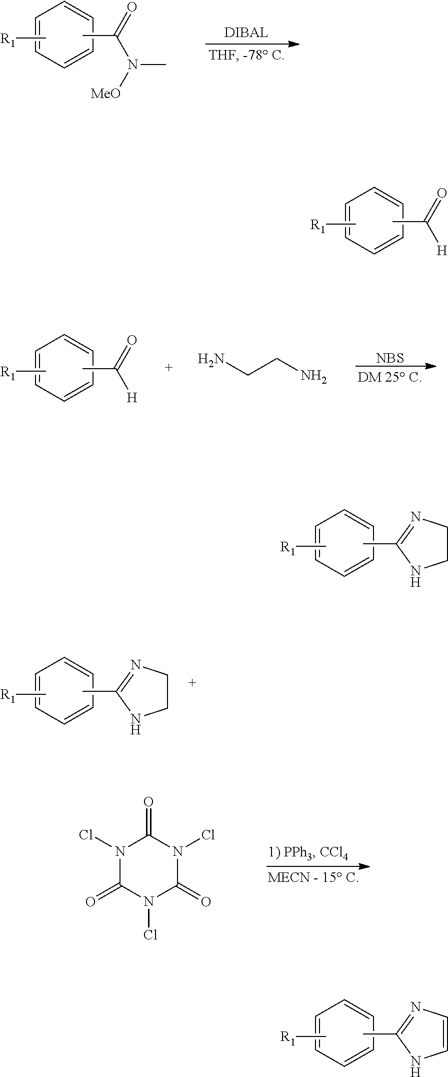

Nitrogen coupled imidazoles can be prepared via copper mediated coupling of imidazole with an aryl iodide.

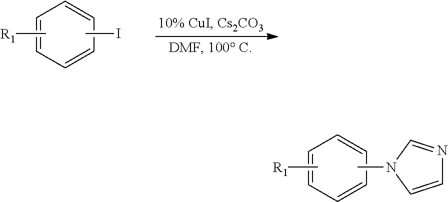

In some aspects, compounds can be prepared via Suzuki coupling with the appropriate boronic acids with a halogenated substrate.

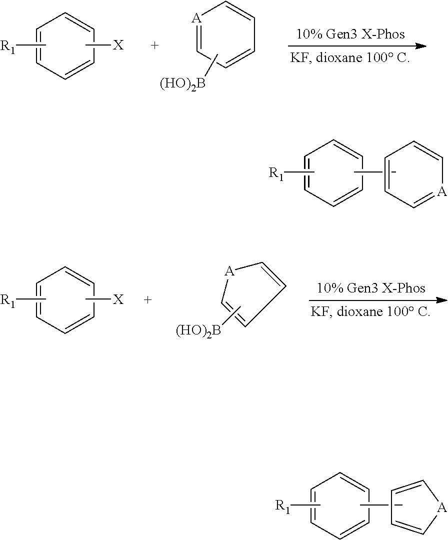

### Pharmaceutical Formulations

In various aspects, the present disclosure relates to pharmaceutical compositions comprising a therapeutically effective amount of a disclosed compound, or a pharmaceutically acceptable salt thereof, and a pharmaceutically acceptable carrier. In a further aspect, the present disclosure relates to pharmaceutical compositions comprising a therapeutically effective amount of at least one disclosed compound or at least one disclosed product of a method of making a compound, as well as pharmaceutically acceptable salt, hydrate, solvate, or polymorph forms of the disclosed compound or the disclosed product of a method of making compound.

As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitonealy, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, a disclosed compound comprising an acidic group or moiety, e.g., a carboxylic acid group, can be used to prepare a pharmaceutically acceptable salt. For example, such a disclosed compound may comprise an isolation step comprising treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed compound comprising a protonatable group or moiety, e.g., an amino group, can be used to prepare a pharmaceutically acceptable salt. For example, such a disclosed compound may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with a basic reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids which can be used to prepare the pharmaceutically acceptable acid-addition salts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C₁-C₁₂-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulphoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C₁-C₆-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik and angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present disclosure may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present disclosure include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present disclosure therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present disclosure include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the disclosure formulated in a pharmaceutically acceptable carrier may also be prepared, placed in an appropriate container, and labeled for treatment of an indicated condition.

Another patch system configuration which can be used by the present disclosure is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present disclosure is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The disclosed pharmaceutical compositions can further comprise other therapeutically active compounds or active agents, which are usually applied in the treatment of the above mentioned pathological or clinical conditions.

In some aspects, the second active agent is a cancer therapeutic. In some aspects, the cancer therapeutic is selected from the group consisting of an antimetabolite, an alkylating agent, interleukin-2, a therapeutic antibody, radiation, and estrogen blockers.

By the phrase “cancer therapeutic” is meant an agent that is administered to a subject for the purpose of treating or reducing the progression of cancer in a mammal. Non-limiting examples of cancer therapeutics can include those that induce cancer cell death (e.g., cancer cell apoptosis) in a mammal. In some embodiments, a cancer therapeutic can reduce the rate of cancer cell division (e.g., reduce the rate of tumor mass growth) or tumor metastasis in a mammal (e.g., as compared to a similar subject having the same type of cancer and receiving no treatment or a different treatment). Non-limiting examples of cancer therapeutics include antimetabolites, alkylating agents, inter leukin-2, and therapeutic antibodies (e.g., trastuzumab). Exemplary cancer therapeutics are described herein. Additional examples of cancer therapeutics are known in the art.

Examples of cancer therapeutics include, without limitation, an antimetabolite, an alkylating agent, interleukin-2, a therapeutic antibody, radiation, or hormone deprivation therapy (e.g., androgen deprivation therapy and estrogen blockers (e.g., tamoxifen, toremifene, fluvestrant, letrozole, anastrozole, exemestane, goserelin, leuprolide, and megestrol acetate). Non-limiting examples of antimetabolites include methotrexate, trimetrexate, pentostatin, cytarabine, fludarabine phosphate, hydroxyurea, fluorouracil, floxuridine, chlorodeoxyadenosine, gemcitabine, thioguanine, and 6-mercaptopurine. Non-limiting examples of alkylating agents include lomustine, carmustine, streptozocin, mechlorethamine, melphalan, uracil nitrogen mustard, chlorambucil, cyclophosphamide, iphosphamide, cisplatin, carboplatin, mitomycin, thiotepa, dacarbazin, procarbazine, hexamethyl melamine, triethylene melamine, busulfan, pipobroman, and mitotane. Non-limiting examples of therapeutic antibodies include ipilimumab and trastuzumab. Additional exemplary cancer therapeutics include bleomycin, topotecan, irinotecan, camptothecin, daunorubicin, doxorubicin, idarubicin, mitoxantrone, teniposide, etoposide, dactinomycin, mithramycin, vinblastine, vincristine, navelbine, paclitaxel, and docetaxel. In some embodiments, a subject is identified as having ovarian cancer (e.g., using the diagnostic methods described herein) and administered a cancer therapeutic selected from the group of doxorubicin and topotecan. One or more (e.g., two, three, four, or five) cancer therapeutics can be administered to the subject.

The therapeutic treatment can be administered by a health care professional (e.g., a physician, a nurse, or a physician's assistant). The treatment can be administered in a patient's home or in a heath care facility (e.g., a hospital or a clinic). The one or more cancer therapeutics can be administered orally, subcutaneously, intramuscularly, intravenously, intraarterially, intrathecally, or intraperitoneally.

The dosage and selection of the cancer therapeutic can be determined by a health care professional based on known in the art. See, e.g., Abraham et al, The Bethesda Handbook of Clinical Oncology (Lippincott Williams & Wilkins; Third edition, Sep. 4, 2009); Casciato and Territo, Manual of Clinical Oncology (Lippincott Manual Series) (Lippincott Williams & Wilkins; Sixth, North American Edition, Sep. 5, 2008); Haffty and Wilson, Handbook of Radiation Oncology: Basic Principles and Clinical Protocols, (Jones & Bartlett Publishers; 1st Edition, Jul. 23, 2008); and Abeloff et al, Abeloff s Clinical Oncology: Expert Consult (Churchill Livingstone; 4th Edition, May 21, 2008); Feig et al, The M.D. Anderson Surgical Oncology Handbook (Lippincott Williams & Wilkins; 4th Edition (Jun. 21, 2006). For example, a single dose of a cancer therapeutic can contain between 1 mg to 500 mg of the therapeutic agent (e.g., between 10 mg and 400 mg, between 10 mg and 300 mg, between 1 mg and 200 mg, between 1 mg and 100 mg, between 1 mg and 50 mg, or between 1 mg and 25 mg).

The one or more cancer therapeutic can be administered to the subject with a frequency of at least once a day, at least twice a day, at least once a week, at least once every two weeks, at least once every month, or at least once every two months. In some embodiments, the one or more cancer therapeutics can be administered to the subject for a treatment period of at least one day (e.g., at least two days, at least three days, at least four days, at least five days, at least six days, at least one week, at least two weeks, or at least one month). Methods for Selecting a Subject for a Clinical Trial

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

As already mentioned, the present disclosure also relates to a pharmaceutical composition comprising a disclosed compound, a product of a disclosed method of making, a pharmaceutically acceptable salt, a hydrate thereof, a solvate thereof, a polymorph thereof, and one or more other drugs in the treatment, prevention, control, amelioration, or reduction of risk of diseases or conditions for a disclosed compound or the other drugs may have utility as well as to the use of such a composition for the manufacture of a medicament. The present disclosure also relates to a combination of disclosed compound, a product of a disclosed method of making, a pharmaceutically acceptable salt, a hydrate thereof, a solvate thereof, a polymorph thereof, and an anti-microbial agent. The present disclosure also relates to such a combination for use as a medicine. The present disclosure also relates to a product comprising (a) disclosed compound, a product of a disclosed method of making, a pharmaceutically acceptable salt, a hydrate thereof, a solvate thereof, a polymorph thereof, and (b) an additional therapeutic agent that has anti-microbial activity, as a combined preparation for simultaneous, separate or sequential use in the treatment or prevention of a condition in a mammal, including a human, the treatment or prevention of which is affected or facilitated by the modulatory effect of the disclosed compound and the additional therapeutic agent. The different drugs of such a combination or product may be combined in a single preparation together with pharmaceutically acceptable carriers or diluents, or they may each be present in a separate preparation together with pharmaceutically acceptable carriers or diluents.

### Methods of Use

In various aspects, the present disclosure relates to uses of the disclosed compounds and pharmaceutical formulations thereof. In one aspect, the disclosure relates to use of at least one disclosed compound; or a pharmaceutically acceptable salt, hydrate, solvate, or polymorph thereof. In a further aspect, the compound used is a product of a disclosed method of making.

In one aspect, the disclosure relates to the use of a compound in the manufacture of a medicament for the treatment of infectious diseases, wherein the compound is a disclosed compound; or a pharmaceutically acceptable salt, hydrate, solvate, or polymorph thereof.

In a further aspect, the disclosure relates to the use of a compound in the manufacture of a medicament for the treatment of infectious diseases; wherein the compound is a product of a disclosed method of making; or a pharmaceutically acceptable salt, hydrate, solvate, or polymorph thereof.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, for use as a medicament.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, wherein a pharmaceutically acceptable carrier is intimately mixed with a therapeutically effective amount of the compound or the product of a disclosed method of making.

In some aspects, the methods of use are directed to the treatment of a disease or disorder associated with expression of intrinsically disordered protein p27 in a subject in need of treatment. In some aspects, the disease or disorder is a cancer. The methods can be used in addition to other cancer treatments, e.g. contemporaneous with other cancer treatments, prior to other cancer treatments, or after other cancer treatments. Such treatments are generally known in the art. The cancer can be one that is associated with a mislocalization of the intrinsically disordered protein p27. In some aspects, the cancer is resistant to a conventional anticancer therapy.

In some aspects, the methods of use are directed to promoting reentry into the cell division cycle in a subject in need thereof. In some aspects, the subject has hearing damage or hearing loss and the method comprises enabling a regeneration of hearing in the subject. The regeneration can include improving hearing by partially or completely restoring hearing loss in the subject.

### Aspects of the Disclosure

The present disclosure will be better understood upon reading the following Aspects which should not be confused with the claims. Any of the numbered Aspects below can, in some instances, be combined with other aspects described elsewhere herein even though such combination may not be expressly disclosed as such herein.

Aspect 1. A compound or a pharmaceutically acceptable salt thereof, the compound having a structure according to Formula I where R¹ is a linear or branched alkyl linker, which can be substituted or unsubstituted (e.g. a substituted or unsubstituted C₁-C₇, C₁-C₅, or C₁-C₃ alkyl linker); where each occurrence of R³⁰ and R³¹ is independently a hydrogen, a halo, a cyano, a hydroxyl, —NH₂, or a substituted or unsubstituted alkyl, haloalkyl, alkoxy, or haloalkoxy (e.g. a substituted or unsubstituted C₁-C₇, C₁-C₅, or C₁-C₃ alkyl, haloalkyl, alkoxy, or haloalkoxy); and wherein either (a) R² is a hydrogen, alkyl, or alkoxy (e.g. a substituted or unsubstituted C₁-C₇, C₁-C₅, or C₁-C₃ alkyl or alkoxy) and Ar¹ is selected from the group consisting of

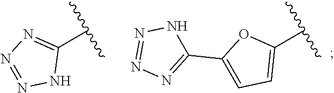 [SMILES: *c1nnn[nH]1 ; *c1ccc(-c2nnn[nH]2)o1]

or (b) R² is —O—R¹—Ar¹; and each occurrence of Ar¹ is independently selected from the group consisting of

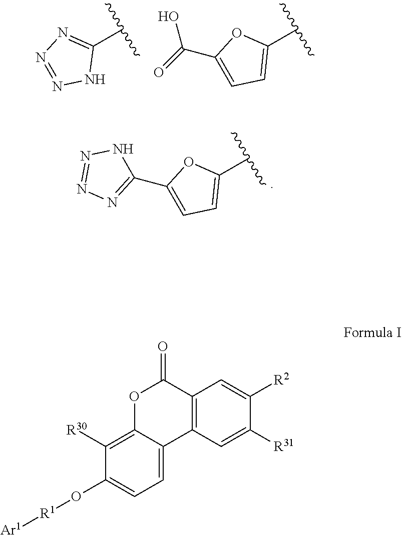

Aspect 2. The compound or pharmaceutically acceptable salt according to any one of Aspects 1-78, wherein R¹ is —CH₂—.

Aspect 3. The compound or pharmaceutically acceptable salt according to any one of Aspects 1-78, wherein R¹ is —C(CH₃)H—.

Aspect 4. The compound or pharmaceutically acceptable salt according to any one of Aspects 1-78, wherein R³⁰ is methyl and R³¹ is hydrogen.

Aspect 5. The compound or pharmaceutically acceptable salt according to any one of Aspects 1-78, wherein R³¹ is methyl and R³⁰ is hydrogen.

Aspect 6. The compound or pharmaceutically acceptable salt according to any one of Aspects 1-78, wherein R³⁰ and R³¹ are methyl.

Aspect 7. The compound or pharmaceutically acceptable salt according to any one of Aspects 1-78, wherein R³⁰ and R³¹ are hydrogen.

Aspect 8. The compound or pharmaceutically acceptable salt according to any one of Aspects 1-78, wherein R² is hydrogen.

Aspect 9. The compound or pharmaceutically acceptable salt according to any one of Aspects 1-78, wherein R² is methyl or methoxy.

Aspect 10. The compound or pharmaceutically acceptable salt according to any one of Aspects 1-78, wherein R² is —O—R¹—Ar¹.

Aspect 11. The compound or pharmaceutically acceptable salt according to any one of Aspects 1-78, wherein Ar¹ is

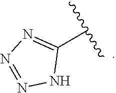 [SMILES: *c1nnn[nH]1]

Aspect 12. The compound or pharmaceutically acceptable salt according to any one of Aspects 1-78, wherein R² is hydrogen.

Aspect 13. The compound or pharmaceutically acceptable salt according to any one of Aspects 1-78, wherein R² is methyl or methoxy.

Aspect 14. The compound or pharmaceutically acceptable salt according to any one of Aspects 1-78, wherein R² is —O—R¹—Ar¹.

Aspect 15. The compound or pharmaceutically acceptable salt according to any one of Aspects 1-78, wherein Ar¹ is

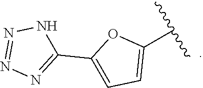 [SMILES: *c1ccc(-c2nnn[nH]2)o1]

Aspect 16. The compound or pharmaceutically acceptable salt according to any one of Aspects 1-78, wherein R² is hydrogen.

Aspect 17. The compound or pharmaceutically acceptable salt according to any one of Aspects 1-78, wherein R² is methyl or methoxy.

Aspect 18. The compound or pharmaceutically acceptable salt according to any one of Aspects 1-78, wherein R² is —O—R¹—Ar¹.

Aspect 19. The compound or pharmaceutically acceptable salt according to any one of Aspects 1-78, wherein Ar¹ is

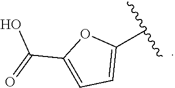 [SMILES: *c1ccc(C(=O)O)o1]

Aspect 20. The compound or pharmaceutically acceptable salt according to any one of Aspects 1-78, wherein the compound has a structure according to any one of the following formulas

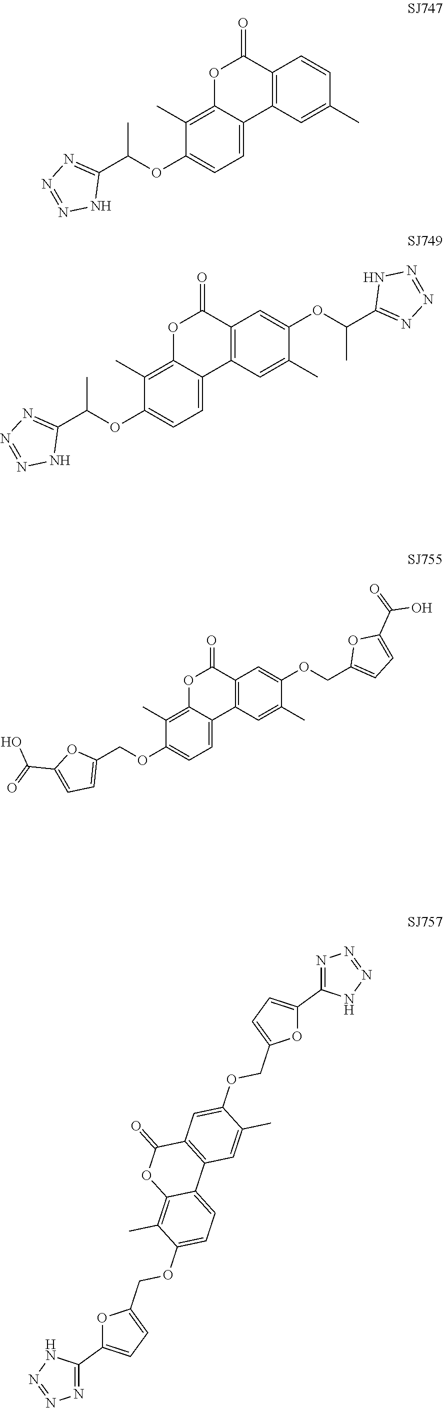

Aspect 21. A compound or a pharmaceutically acceptable salt thereof, wherein the compound has a structure according to Formula II where each occurrence of R³⁰ and R³¹ is independently a hydrogen, a halo, a cyano, a hydroxyl, —NH₂, or a substituted or unsubstituted alkyl, haloalkyl, alkoxy, or haloalkoxy (e.g. a substituted or unsubstituted C₁-C₇, C₁-C₅, or C₁-C₃ alkyl, haloalkyl, alkoxy, or haloalkoxy); where R² is a hydrogen, a halo, a cyano, a hydroxyl, —NH₂, or a alkyl, haloalkyl, alkoxy, or haloalkoxy (e.g. a substituted or unsubstituted C₁-C₁₂, C₁-C₇, C₁-C₅, or C₁-C₃ alkyl, haloalkyl, alkoxy, or haloalkoxy) or —O—R¹—Ar²¹—Ar²²; where each occurrence of R¹ and R⁴ is independently a linear or branched chain alkyl linker which can be substituted or unsubstituted (e.g. a substituted or unsubstituted C₁-C₁₅, C₁-C₁₂, C₁-C₇, C₁-C₅, or C₁-C₃ alkyl linker); where each occurrence of Ar²¹ is independently a bond or selected from the group

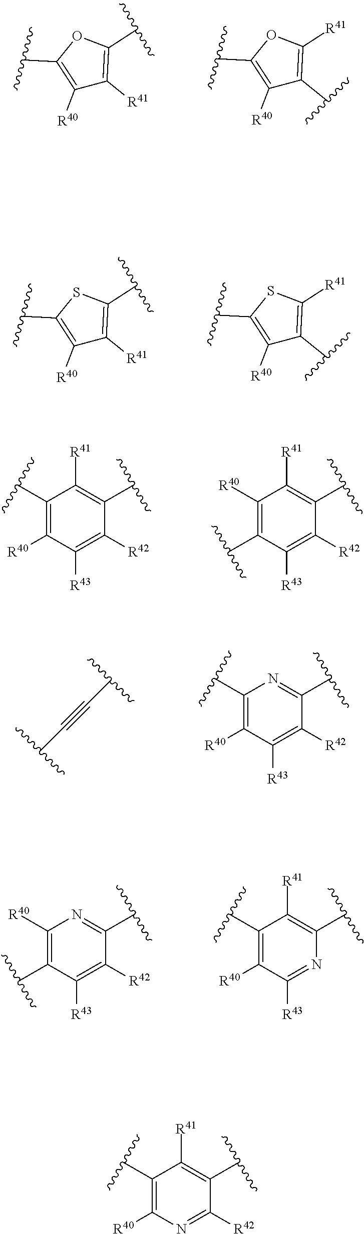

where each occurrence of R⁴⁰, R⁴¹, R⁴², and R⁴³ is independently a hydrogen, a halo, a cyano, a hydroxyl, —NH₂, or a substituted or unsubstituted alkyl, haloalkyl, alkoxy, or haloalkoxy (e.g. a substituted or unsubstituted C₁-C₇, C₁-C₅, or C₁-C₃ alkyl, haloalkyl, alkoxy, or haloalkoxy); and where each occurrence of Ar²² is independently selected from the group

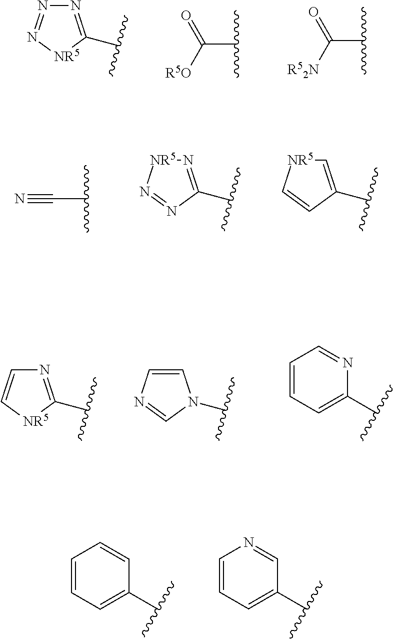

where each occurrence of R⁵ is independently hydrogen or a substituted or unsubstituted alkyl, haloalkyl, alkoxy, or haloalkoxy (e.g. a C₁-C₁₂, C₁-C₇, C₁-C₅, or C₁-C₃ alkyl, haloalkyl, alkoxy, or haloalkoxy).

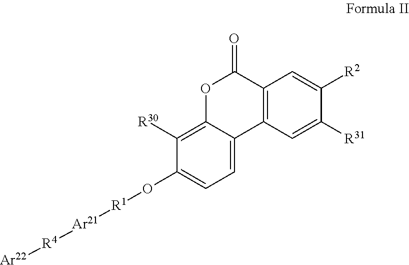

Aspect 22. The compound or pharmaceutically acceptable salt according to any one of Aspects 1-78, wherein R¹ is —CH₂—.

Aspect 23. The compound or pharmaceutically acceptable salt according to any one of Aspects 1-78, wherein R¹ is —C(CH₃)H—.

Aspect 24. The compound or pharmaceutically acceptable salt according to any one of Aspects 1-78, wherein each occurrence of R¹ is a linear or branched, C₁-C₃ alkyl linker.

Aspect 25. The compound or pharmaceutically acceptable salt according to any one of Aspects 1-78, wherein R⁴ is —CH₂—.

Aspect 26. The compound or pharmaceutically acceptable salt according to any one of Aspects 1-78, wherein R⁴ is —C(CH₃)H—.

Aspect 27. The compound or pharmaceutically acceptable salt according to any one of Aspects 1-78, wherein each occurrence of R⁴ is a linear or branched, C₁-C₃ alkyl linker.

Aspect 28. The compound or pharmaceutically acceptable salt according to any one of Aspects 1-78, wherein R³⁰ is methyl and R³¹ is hydrogen.

Aspect 29. The compound or pharmaceutically acceptable salt according to any one of Aspects 1-78, wherein R³¹ is methyl and R³⁰ is hydrogen.

Aspect 30. The compound or pharmaceutically acceptable salt according to any one of Aspects 1-78, wherein R³⁰ and R³¹ are methyl.

Aspect 31. The compound or pharmaceutically acceptable salt according to any one of Aspects 1-78, wherein R³⁰ and R³¹ are hydrogen.

Aspect 32. The compound or pharmaceutically acceptable salt according to any one of Aspects 1-78, wherein one or both of R³⁰ and R³¹ is hydrogen.

Aspect 33. The compound or pharmaceutically acceptable salt according to any one of Aspects 1-78, wherein one or both of R³⁰ and R³¹ is halo.

Aspect 34. The compound or pharmaceutically acceptable salt according to any one of Aspects 1-78, wherein one or both of R³⁰ and R³¹ is cyano.

Aspect 35. The compound or pharmaceutically acceptable salt according to any one of Aspects 1-78, wherein one or both of R³⁰ and R³¹ is hydroxyl.

Aspect 36. The compound or pharmaceutically acceptable salt according to any one of Aspects 1-78, wherein one or both of R³⁰ and R³¹ is —NH₂.

Aspect 37. The compound or pharmaceutically acceptable salt according to any one of Aspects 1-78, wherein one or both of R³⁰ and R³¹ is a C₁-C₃ alkyl.

Aspect 38. The compound or pharmaceutically acceptable salt according to any one of Aspects 1-78, wherein one or both of R³⁰ and R³¹ is C₁-C₃ haloalkyl.

Aspect 39. The compound or pharmaceutically acceptable salt according to any one of Aspects 1-78, wherein one or both of R³⁰ and R³¹ is C₁-C₃ alkoxy.

Aspect 40. The compound or pharmaceutically acceptable salt according to any one of Aspects 1-78, wherein one or both of R³⁰ and R³¹ is C₁-C₃ haloalkoxy.

Aspect 41. The compound or pharmaceutically acceptable salt according to any one of Aspects 1-78, wherein R² is hydrogen.

Aspect 42. The compound or pharmaceutically acceptable salt according to any one of Aspects 1-78, wherein R² is halo.

Aspect 43. The compound or pharmaceutically acceptable salt according to any one of Aspects 1-78, wherein R² is cyano.

Aspect 44. The compound or pharmaceutically acceptable salt according to any one of Aspects 1-78, wherein R² is hydroxyl.

Aspect 45. The compound or pharmaceutically acceptable salt according to any one of Aspects 1-78, wherein R² is —NH₂.

Aspect 46. The compound or pharmaceutically acceptable salt according to any one of Aspects 1-78, wherein R² is a C₁-C₃ alkyl.

Aspect 47. The compound or pharmaceutically acceptable salt according to any one of Aspects 1-78, wherein R² is a C₁-C₃ haloalkyl.

Aspect 48. The compound or pharmaceutically acceptable salt according to any one of Aspects 1-78, wherein R² is a C₁-C₃ alkoxy.

Aspect 49. The compound or pharmaceutically acceptable salt according to any one of Aspects 1-78, wherein R² is a C₁-C₃ haloalkoxy.

Aspect 50. The compound or pharmaceutically acceptable salt according to any one of Aspects 1-78, wherein R² is —O—R¹—Ar²¹—Ar²².

Aspect 51. The compound or pharmaceutically acceptable salt according to any one of Aspects 1-78, wherein each occurrence of R⁴⁰, R⁴¹, R⁴², and R⁴³ is hydrogen or hydroxyl.

Aspect 52. The compound or pharmaceutically acceptable salt according to any one of Aspects 1-78, wherein at least one occurrence of R⁴⁰, R⁴¹, R⁴², and R⁴³ is methyl and the remaining occurrences are either hydrogen or hydroxyl.

Aspect 53. The compound or pharmaceutically acceptable salt according to any one of Aspects 1-78, wherein R⁵ is hydrogen.

Aspect 54. The compound or pharmaceutically acceptable salt according to any one of Aspects 1-78, wherein R⁵ is hydroxyl.

Aspect 55. The compound or pharmaceutically acceptable salt according to any one of Aspects 1-78, wherein R⁵ is methyl.

Aspect 56. The compound or pharmaceutically acceptable salt according to any one of Aspects 1-78, wherein Ar²¹ is

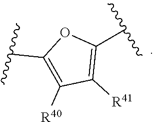

Aspect 57. The compound or pharmaceutically acceptable salt according to any one of Aspects 1-78, wherein Ar²¹ is

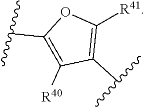

Aspect 58. The compound or pharmaceutically acceptable salt according to any one of Aspects 1-78, wherein Ar²¹ is

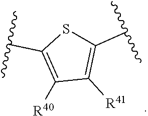

Aspect 59. The compound or pharmaceutically acceptable salt according to any one of Aspects 1-78, wherein Ar²¹ is

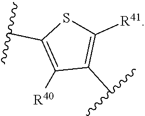

Aspect 60. The compound or pharmaceutically acceptable salt according to any one of Aspects 1-78, wherein Ar²¹ is

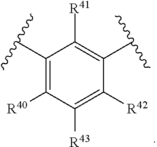

Aspect 61. The compound or pharmaceutically acceptable salt according to any one of Aspects 1-78, wherein Ar²¹ is

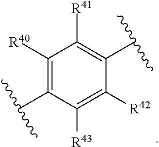

Aspect 62. The compound or pharmaceutically acceptable salt according to any one of Aspects 1-78, wherein Ar²¹ is

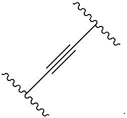 [SMILES: *C#C*]

Aspect 63. The compound or pharmaceutically acceptable salt according to any one of Aspects 1-78, wherein Ar²¹ is

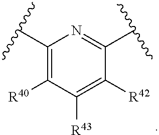

Aspect 64. The compound or pharmaceutically acceptable salt according to any one of Aspects 1-78, wherein Ar²¹ is

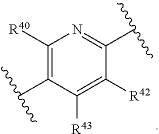

Aspect 65. The compound or pharmaceutically acceptable salt according to any one of Aspects 1-78, wherein Ar²¹ is

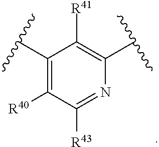

Aspect 66. The compound or pharmaceutically acceptable salt according to any one of Aspects 1-78, wherein Ar²¹ is

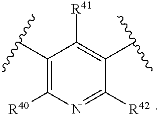

Aspect 67. The compound or pharmaceutically acceptable salt according to any one of Aspects 1-78, wherein Ar²² is

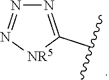

Aspect 68. The compound or pharmaceutically acceptable salt according to any one of Aspects 1-78, wherein Ar²² is

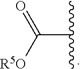

Aspect 69. The compound or pharmaceutically acceptable salt according to any one of Aspects 1-78, wherein Ar²² is

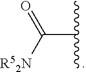

Aspect 70. The compound or pharmaceutically acceptable salt according to any one of Aspects 1-78, wherein Ar²² is

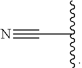 [SMILES: *C#N]

Aspect 71. The compound or pharmaceutically acceptable salt according to any one of Aspects 1-78, wherein Ar²² is

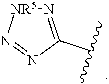

Aspect 72. The compound or pharmaceutically acceptable salt according to any one of Aspects 1-78, wherein Ar²² is

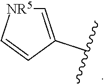

Aspect 73. The compound or pharmaceutically acceptable salt according to any one of Aspects 1-78, wherein Ar²² is

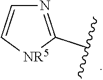

Aspect 74. The compound or pharmaceutically acceptable salt according to any one of Aspects 1-78, wherein Ar²² is

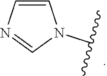 [SMILES: *n1ccnc1]

Aspect 75. The compound or pharmaceutically acceptable salt according to any one of Aspects 1-78, wherein Ar²² is

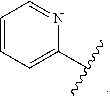 [SMILES: *c1ccccn1]

Aspect 76. The compound or pharmaceutically acceptable salt according to any one of Aspects 1-78, wherein Ar²² is

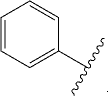 [SMILES: *c1ccccc1]

Aspect 77. The compound or pharmaceutically acceptable salt according to any one of Aspects 1-78, wherein Ar²² is

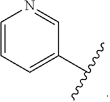 [SMILES: *c1cccnc1]

Aspect 78. The compound or pharmaceutically acceptable salt according to any one of Aspects 1-78, wherein the compound has a structure according to any one of the following formulas

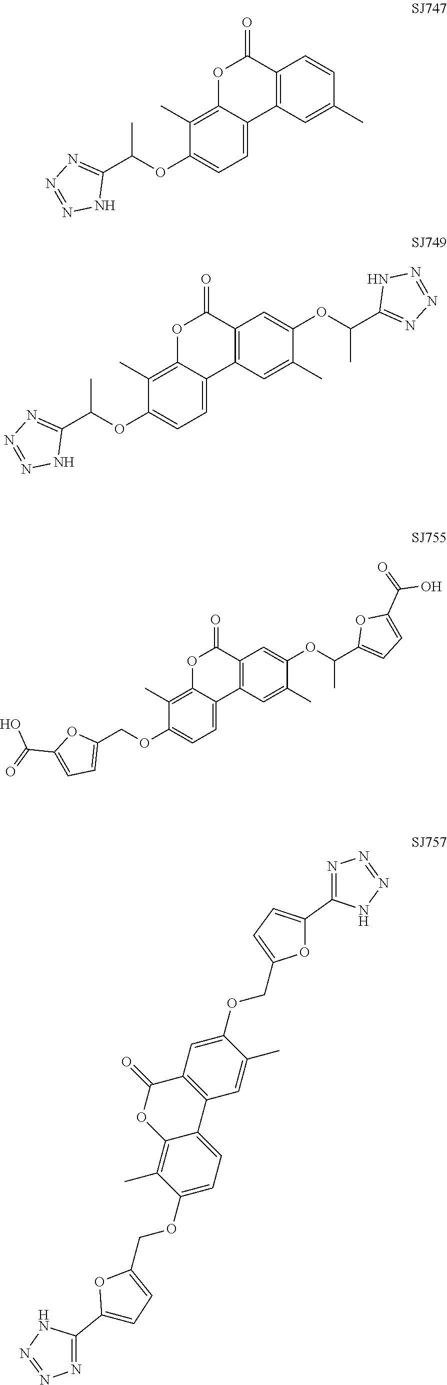 [SMILES: Cc1ccc2c(=O)oc3c(C)c(OC(C)c4nnn[nH]4)ccc3c2c1 ; Cc1cc2c(cc1OC(C)c1nnn[nH]1)c(=O)oc1c(C)c(OC(C)c3nnn[nH]3)ccc12 ; Cc1cc2c(cc1OC(C)c1ccc(C(=O)O)o1)c(=O)oc1c(C)c(OCc3ccc(C(=O)O)o3)ccc12 ; Cc1cc2c(cc1OCc1ccc(-c3nnn[nH]3)o1)c(=O)oc1c(C)c(OCc3ccc(-c4nnn[nH]4)o3)ccc12]

Aspect 79. The compound or pharmaceutically acceptable salt according to any one of Aspects 1-78, comprising an acid addition salt derived from an acid selected from 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, hydrochloric acid, isethionic, isobutyric acid, lactic acid, lactobionic acid, lauric acid, maleic acid, malic acid, malonic acid, mandelic acid, methanesulfonic acid, mucic, naphthalene-1,5-disulfonic acid, naphthalene-2-sulfonic acid, nicotinic acid, nitric acid, oleic acid, oxalic acid, palmitic acid, pamoic acid, pantothenic, phosphoric acid, proprionic acid, pyroglutamic acid, salicylic acid, sebacic acid, stearic acid, succinic acid, sulfuric acid, tartaric acid, thiocyanic acid, toluenesulfonic acid, trifluoroacetic, and undecylenic acid.

Aspect 80. The compound or pharmaceutically acceptable salt according to any one of Aspects 1-78, comprising a base addition salt derived from an alkali metal or alkaline earth metal hydroxide, e.g. calcium hydroxide, magnesium hydroxide, sodium hydroxide, lithium hydroxide, zinc hydroxide, potassium hydroxide, or iron hydroxide.

Aspect 81. A pharmaceutical formulation comprising a therapeutically effective amount of a compound or pharmaceutically acceptable salt according to any one of Aspects 1-80 and a pharmaceutically acceptable carrier.

Aspect 82. The pharmaceutical formulation according to any one of Aspects 81-87, wherein the compound has a structure according to any one of the following formulas

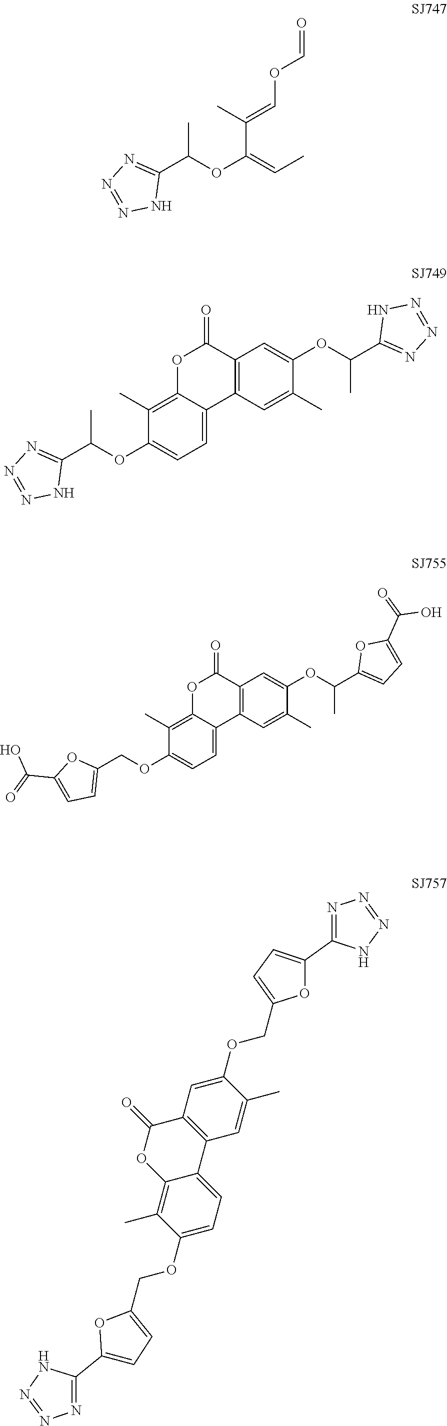 [SMILES: C/C=C(OC(C)c1nnn[nH]1)\C(C)=C\OC=O ; Cc1cc2c(cc1OC(C)c1nnn[nH]1)c(=O)oc1c(C)c(OC(C)c3nnn[nH]3)ccc12 ; Cc1cc2c(cc1OC(C)c1ccc(C(=O)O)o1)c(=O)oc1c(C)c(OCc3ccc(C(=O)O)o3)ccc12 ; Cc1cc2c(cc1OCc1ccc(-c3nnn[nH]3)o1)c(=O)oc1c(C)c(OCc3ccc(-c4nnn[nH]4)o3)ccc12]

Aspect 83. The pharmaceutical formulation according to any one of Aspects 81-87, wherein the pharmaceutical composition is a solid dosage form selected from the group consisting of a capsule, a tablet, a pill, a powder, a granule, an effervescing granule, a gel, a paste, a troche, and a pastille.

Aspect 84. The pharmaceutical formulation according to any one of Aspects 81-87, wherein the pharmaceutical composition is liquid dosage form selected from the group consisting of an emulsion, a solution, a suspension, a syrup, and an elixir.

Aspect 85. The pharmaceutical formulation according to any one of Aspects 81-87, further comprising a second active agent.

Aspect 86. The pharmaceutical formulation according to any one of Aspects 81-87, wherein the second active agent is a cancer therapeutic.

Aspect 87. The pharmaceutical formulation according to any one of Aspects 81-87, wherein the cancer therapeutic is selected from the group consisting of an antimetabolite, an alkylating agent, interleukin-2, a therapeutic antibody, radiation, and estrogen blockers.

Aspect 88. A method for the treatment of a disease or disorder associated with expression of intrinsically disordered protein p27 in a subject in need of treatment, the method comprising administering a therapeutically effective amount of a compound or a pharmaceutically acceptable salt according to any one of Aspects 1-80 or a pharmaceutical formulation according to any one of Aspects 81-87.

Aspect 89 The method according to any one of Aspects 88-93, wherein the disease or disorder is a cancer.

Aspect 90 The method according to any one of Aspects 88-93, wherein the cancer is associated with a mislocalization of the intrinsically disordered protein p27.

Aspect 91 The method according to any one of Aspects 88-93, wherein the cancer is resistant to an anticancer therapy.

Aspect 92 A method of promoting reentry into the cell division cycle in a subject in need thereof, the method comprising administering a therapeutically effective amount of a compound or a pharmaceutically acceptable salt according to any one of Aspects 1-80 or a pharmaceutical formulation according to any one of Aspects 81-87.

Aspect 93. The method according to any one of Aspects 88-93, wherein the subject has hearing damage or hearing loss and the method comprises enabling a regeneration of hearing in the subject.

### Examples

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Methods

### Preparation of Proteins

The p27 constructs were expressed in E. Coli (BL21/DE3) with an N-terminal 6×His affinity tag after sub-cloning into pET28a (Novagen) using established procedures (Lacy, E. R., et al., Nat Struct Mol Biol, 2004. 11(4): p. 358-64.). This included p27-KID (residues 22-105 of human p27) and p27-D2 (residues 58-105 of human p27) and the following mutants: W60A, W76A, W60A-W76A. Isotope-labeled proteins (¹⁵N and ¹³C/¹⁵N) were expressed in a MOPS buffer-based minimal media using established procedures (Grimmler, M., et al., Cell, 2007. 128(2): p. 269-80). All p27 constructs were purified by nickel affinity chromatography, digested with thrombin to remove the 6×His tag, and further purified using reverse-phase high performance liquid chromatography (HPLC) using a C4 column (Vydac) and 0.1% trifluoroacetic acid-containing water/acetonitrile solvent system. Protein concentrations were determined by UV absorbance at 280 nm under denaturing conditions using a molar extinction coefficient of 15,470 M⁻¹ cm⁻¹ for p27-KID and p27-D2; 9,970 M⁻¹ cm⁻¹ for p27 variants with a single tryptophan residue; and 4,470 M⁻¹ cm⁻¹ for p27 variants without a tryptophan residue. Full length human Cdk2, active Cdk2 (phosphorylated at threonine 160), and truncated human cyclin A (residues 173-432) were expressed and purified using established protocols (Lacey et al.; Bowman, P., et al., Biochim Biophys Acta, 2006. 1764(2): p. 182-9).

### SAR-by-Analogs: Cheminformatics Analysis

Based on the central cores of Group 1 (G1.1, G1.2, and G1.3) and Group 2 (G2) hits, respectively, substructure searches were performed in SciFinder and selected compounds were purchased from commercial vendors: Vitas-M, Vitascreen, LLC, University of Illinois Urbana-Champaign Research Park, 2001 South First Street, Suite 201, Champaign, Ill., 61820; Ambinter, Ambinter c/o Greenpharma, 3, allée du titane, 45100 Orléans, FRANCE; ChemBridge, ChemBridge Corporation, 11199 Sorrento Valley Rd., Suite 206, San Diego, Calif. 92121, USA; PrincetonBio, Princeton BioMolecular Research, Inc., 475 Wall Street, Princeton, N.J. 08540, USA; Alinda, SRC Alinda, Kuskovskaya Street, 20A, entrance 2B, office 409 111141, Moscow, Rusia; Specs, Specs US Compound Management Facility, 14900 Burbridge Road SE, Cumberland, Md. 21502, USA; and Maybridge, Fisher Scientific UK Ltd, T/A Maybridge, Bishop Meadow Road, Loughborough, Leicestershire, LE11 5RG, UK. The Group 1 and Group 2 analog types are defined in [Iconaru, L. I., et al., Discovery of Small Molecules that Inhibit the Disordered Protein, p27(Kip1). Sci Rep, 2015. 5: p. 15686].

### Chemical Synthesis of G1.1 Compounds

All materials were purchased from commercial suppliers and used without further purification. Pre-purification and QC analyses were done on a Waters Acquity UPLC/PDA/ELSD/MS system carried out with a BEH C18 2.1×50 mm column using gradient elution. Purification of compounds was carried out by normal phase column chromatography using pre-pack SNAP silica cartridges on a Biotage Isolera system. Reported yields were not optimized. Structures were determined by NMR spectroscopy and purity was determined by LC-MS/ELSD. NMR spectra (1D ¹H, and ¹³C, respectively, and 2D ¹H-¹³C HSQC and ¹H-¹³C HMBC, respectively) were recorded on a Bruker 600 MHz spectrometer equipped with TCI cryogenic gradient probe and processed and analyzed using Bruker Topspin software.

General procedure for generation of 6H-benzo[c]chromen-6-one tricyclic core (1-3). In a 5 mL glass vial, resorcinol derivatives (2 mmol) and o-bromobenzoic derivatives (1 mmol) were mixed with water (0.7 mL) and NaOH (5N, 0.7 mL) and heated at 100° C. for 15 minutes. CuSO₄ (10% aqueous solution, 0.2 mL) was added to the reaction mixture, the vial was sealed and further heated at 100° C. for 6 hours. The reaction mixture was cooled on ice and the precipitate was filtered, washed with cold water, and dried in vacuo.

3-hydroxy-4,9-dimethyl-6H-benzo[c]chromen-6-one (1) (yield 51%)¹H-NMR δ 2.214 (3H, s), 2.514 (3H, s), 6.898 (1H, d, ³J=8.63 Hz), 7.381 (1H, d, ³J=7.94 Hz), 7.998 (1H, d, ³J=8.63 Hz), 8.083 (1H, s), 8.092 (1H, d, ³J=7.94), 10.27 (1H, s). ¹³C-NMR δ 8.903, 22.326, 109.758, 111.916, 112.633, 116.8, 121.808, 122.291, 129.325, 130.233, 136.163, 146.568, 151.035, 158.492, 161.318. MS (ES+): m/z=241.21.

3-hydroxy-8-methoxy-4,9-dimethyl-6H-benzo[c]chromen-6-one (2) (yield 70%)¹H-NMR δ 2.216 (3H, s), 2.358 (3H, s), 3.933 (3H, s), 6.885 (1H, d, ³J=8.6 Hz), 7.56 (1H, s), 7.928 (1H, d, ³J=8.6 Hz), 8.095 (1H, s), 10.08 (1H, s). ¹³C-NMR δ 8.904, 17.401, 56.273, 108.69, 110.89, 111.833, 112.525, 118.152, 121.159, 124.421, 129.41, 136.3, 150.179, 157.276, 157.584, 161.306. MS (ES+): m/z=271.21.

3-hydroxy-8-methoxy-1,4,9-trimethyl-6H-benzo[c]chromen-6-one (3) (yield 59%) ¹H-NMR δ 2.193 (3H, s), 2.373 (3H, s), 2.744 (3H, s), 3.945 (3H, s), 6.732 (1H, s), 7.67 (1H, s), 8.139 (1H, s), 10.05 (1H, s). ¹³C-NMR δ 9.094, 17.719, 25.681, 56.2, 109.246, 109.39, 109.933, 116.486, 119.255, 128.158, 130.414, 133.485, 135.104, 150.986, 156.196, 156.69, 161.16. MS (ES+): m/z=285.11.

Synthesis of 2-((4,9-dimethyl-6-oxo-6H-benzo[c]chromen-3-yl)oxy)propanenitrile (4) In a 5 mL glass vial, under nitrogen, derivative 1 (0.5 mmol) was mixed with potassium carbonate (0.6 mmol) and 1 mL anhydrous DMF and stirred for 15 minutes at 70° C. 2-chloropropanenitrile (1 mmol) was added and the reaction mixture was further stirred for 6 hours at 70° C. The reaction was subsequently cooled, diluted with EtOAc (10 mL) and washed three times with water (3×10 mL). The organic phase was dried over anhydrous MgSO4, filtered, and concentrated under reduced pressure. Pure compound was obtained by flash chromatography using an EtOAc/hexane/MeOH gradient (yield 68%). ¹H-NMR δ 1.786 (3H, d, ³J=6.57 Hz), 2.284 (3H, s), 2.545 (3H, s), 5.622 (1H, q, ³J=6.57 Hz), 7.281 (1H, d, ³J=8.84 Hz), 7.475 (1H, d, ³J=8.01 Hz), 8.134 (1H, d, ³J=8.01 Hz), 8.238 (1H, s), 8.279 (1H, d, ³J=8.84 Hz). ¹³C-NMR δ 9.013, 19.909, 22.317, 63.567, 110.802, 113.365, 115.393, 117.619, 119.619, 122.367, 123.047, 130.303, 130.408, 135.2, 146.852, 150.509, 155.942, 160.862. MS (ES+): m/z=294.26.

Synthesis of 3-(1-(1H-tetrazol-5-yl)ethoxy)-4,9-dimethyl-6H-benzo[c]chromen-6-one (SJ982747; SJ747) In a 5 mL glass vial, under nitrogen, nitrile derivative 4 (0.3 mmol) was mixed with NaN₃ (0.6 mmol), NH₄Cl (0.6 mmol), and anhydrous DMF (2 mL) and stirred overnight at 100° C. The reaction was cooled down and iced water (3 mL) added. The reaction mixture was adjusted to pH 11 with NaOH and filtered through celite. The product separated upon acidification to pH 2 as an off-white solid (yield 65%). ¹H-NMR δ 1.762 (3H, d, ³J=6.47 Hz), 2.234 (3H, s), 2.48 (3H, s), 6.08 (1H, q, ³J=6.47 Hz), 7.099 (1H, d), 7.406 (1H, d), 8.069 (3H, m). ¹³C-NMR δ 9.043, 20.647, 22.237, 68.052, 110.939, 112.409, 115.474, 117.222, 121.984, 122.69, 130.211, 130.235, 135.225, 146.956, 150.342, 156.611, 158.041, 161.112. MS (ES+): m/z=337.10.

Synthesis of 3,8-dihydroxy-4,9-dimethyl-6H-benzo[c]chromen-6-one (5) In a two-necked round bottom flask, under nitrogen, methoxy derivative 2 (0.5 mmol) was mixed with anhydrous CH₂Cl₂ (2 mL), and BBr₃ (1 mmol). The reaction mixture was stirred overnight at room temperature. The reaction was diluted with CH₂Cl₂ (3 mL) treated with water (3 mL), and then extracted with EtOAc. The organic phase was dried over anhydrous MgSO4, filtered, and concentrated under reduced pressure. Pure compound was obtained by flash chromatography using an EtOAc/hexane/MeOH gradient (yield 58%). ¹H-NMR δ 2.2 (3H, s), 2.327 (3H, s), 6.86 (1H, d, ³J=8.56 Hz), 7.537 (1H, s), 7.88 (1H, d, ³J=8.56 Hz), 8.013 (1H, s), 9.979 (1H, s), 10.186 (1H, s). ¹³C-NMR δ 8.906, 17.351, 110.427, 111.777, 112.425, 112.961, 118.173, 120.793, 124.492, 127.959, 135.099, 149.893, 155.983, 156.828, 161.29. MS (ES+): m/z=257.15.

Synthesis of 2,2′-((4,9-dimethyl-6-oxo-6H-benzo[c]chromene-3,8-diyl)bis(oxy))-dipropanenitrile (6) In a 5 mL glass vial, under nitrogen, derivative 5 (0.25 mmol) was mixed with potassium carbonate (0.6 mmol) and 1 mL anhydrous DMF and stirred for 15 minutes at 70° C. 2-chloropropanenitrile (1 mmol) was added and the reaction mixture was further stirred for 12 hours at 70° C. The reaction was subsequently cooled, diluted with EtOAc (10 mL) and washed three times with water (3×10 mL). The organic phase was dried over anhydrous MgSO₄, filtered, and concentrated under reduced pressure. Pure compound was obtained by flash chromatography using an EtOAc/hexane/MeOH gradient (yield 61%). ¹H-NMR δ 1.78 (6H, b), 2.268 (3H, s), 2.397 (3H, s), 5.570 (1H, q, ³J=6.59 Hz), 5.677 (1H, q, ³J=6.59 Hz), 7.25 (1H, d, ³J=8.86 Hz), 7.816 (1H, s), 8.177 (1H, d, ³J=8.86 Hz), 8.272 (1H, s). ¹³C-NMR δ 8.964, 17.188, 19.803, 19.91, 63.152, 63.64, 110.96, 111.875, 113.131, 115.44, 119.003, 119.44, 119.602, 121.924, 125.747, 130.048, 137.159, 149.884, 154.926, 155.5, 160.612. MS (ES+): m/z=363.02.

Synthesis of 3,8-bis(1-(1H-tetrazol-5-yl)ethoxy)-4,9-dimethyl-6H-benzo[c]chromen-6-one (SJ982749; SJ749) In a 5 mL glass vial, under nitrogen, bis-nitrile derivative 6 (0.3 mmol) was mixed with NaN₃ (1.2 mmol), NH₄Cl (1.2 mmol), and anhydrous DMF (2 mL) and stirred overnight at 100° C. The reaction was cooled and iced water (3 mL) added. The reaction mixture was adjusted to pH 11 with NaOH and filtered through celite. The product separated upon acidification to pH 2 as a light-yellow precipitate (yield 60%). ¹H-NMR δ 1.758 (3H, d, ³J=6.68 Hz), 1.776 (3H, d, ³J=6.68 Hz), 2.238 (3H, s), 2.379 (3H, s), 6.072 (1H, q, ³J=6.68 Hz), 6.137 (1H, q, ³J=6.68 Hz), 7.098 (1H, d, ³J=8.86 Hz), 7.646 (1H, s), 8.018 (1H, d, ³J=8.86 Hz), 8.149 (1H, s). ¹³C-NMR δ 9.116, 17.465, 20.610, 20.651, 67.814, 68.139, 111.082, 111.796, 112.407, 115.399, 118.711, 121.559, 125.307, 129.416, 137.528, 149.758, 155.545, 156.094, 158.002, 158.171, 160.746. MS (ES+): m/z=449.10.

Synthesis of dimethyl 5,5′-((4,9-dimethyl-6-oxo-6H-benzo[c]chromene-3,8diyl)-bis-(oxy))bis-(methylene))bis(furan-2-carboxylate) (7) In a 5 mL glass vial, under nitrogen, derivative 5 (0.25 mmol) was mixed with potassium carbonate (0.6 mmol) and 1 mL anhydrous DMF and stirred for 15 minutes at 70° C. Methyl 5-(chloromethyl) furan-2-carboxylate (1 mmol) was added and the reaction mixture was further stirred for 12 hours at 70° C. The reaction was subsequently cooled, diluted with EtOAc (10 mL) and washed three times with water (3×10 mL). The organic phase was dried over anhydrous MgSO₄, filtered, and concentrated under reduced pressure. Pure compound was obtained by flash chromatography using an EtOAc/hexane/MeOH gradient (yield 54%). ¹H-NMR δ 2.24 (3H, s), 2.367 (3H, s), 3.833 (6H, s), 5.327 (2H, s), 5.371 (2H, s), 6.834 (2H, d³J=3.06 Hz), 7.252 (1H, d, ³J=8.85 Hz), 7.335 (1H, d, ³J=3.06 Hz), 7.348 (1H, d, ³J=3.06 Hz), 7.776 (1H, s), 8.14 (1H, d, ³J=8.85 Hz), 8.249 (1H, s). ¹³C-NMR δ 8.893, 17.387, 52.524, 62.891, 63.207, 110.012, 110.66, 112.215, 113.04, 113.173, 114.353, 118.727, 119.711, 119.733, 121.611, 125.18, 129.328, 136.762, 144.525, 144.586, 149.705, 154.906, 155.151, 156.524, 157.13, 158.854, 160.922. MS (ES⁺): m/z=533.07.

Synthesis of 5,5′-((4,9-dimethyl-6-oxo-6H-benzo[c]chromene-3,8-diyl)bis(oxy))bis-(methylene))-bis(furan-2-carboxylic acid) (SJ982755; SJ755) In a 10 mL glass vial, derivative 7 (0.15 mmol) was dissolved in MeOH (3 mL) and NaOH (1 mmol) was added. The reaction mixture was stirred at room temperature for 12 hours. Solvent was removed under reduced pressure; the residue was resuspended in water (2 mL), pH adjusted to 11, and solution filtered through celite. The product separated upon acidification to pH 2 as an off-white precipitate (yield 72%). ¹H-NMR δ 2.239 (3H, s), 2.366 (3H, s), 5.3 (2H, s), 5.342 (2H, s), 6.787 (2H, d, ³J=3.18 Hz), 7.223 (1H, d, ³J=3.18 Hz), 7.236 (1H, d, ³J=3.18 Hz), 7.251 (1H, d, ³J=8.84 Hz), 7.771 (1H, s), 8.134 (1H, d, ³J=8.84 Hz), 8.239 (1H, s). ¹³C-NMR δ 8.974, 17.381, 62.959, 63.26, 110.046, 110.653, 112.167, 112.926, 113.039, 114.365, 118.687, 119.043, 119.021, 121.588, 125.137, 129.301, 136.827, 145.788, 145.851, 149.682, 154.265, 154.518, 156.549, 157.161, 159.839, 160.975. MS (ES+): m/z=505.19.

Synthesis of 3,8-dihydroxy-1,4,9-trimethyl-6H-benzo[c]chromen-6-one (8) In a two-necked round bottom flask, under nitrogen, methoxy derivative 3 (0.5 mmol) was mixed with anhydrous CH₂Cl₂ (2 mL), and BBr₃ (1 mmol). The reaction mixture was stirred overnight at room temperature. The reaction was diluted with CH₂Cl₂ (3 mL), treated with water (3 mL), and then extracted with EtOAc. The organic phase was dried over anhydrous MgSO4, filtered, and concentrated under reduced pressure. Pure compound was obtained by flash chromatography using an EtOAc/hexane/MeOH gradient (yield 48%). ¹H-NMR δ 2.169 (3H, s), 2.405 (3H, s), 2.705 (3H, s), 6.699 (1H, s), 7.639 (1H, s), 8.04 (1H, s), 9.862 (1H, s), 10.18 (1H, s). ¹³C-NMR δ 9.08, 17.675, 25.683, 109.697, 109.877, 113.767, 116.251, 119.256, 128.374, 128.909, 131.115, 133.826, 150.662, 155.161, 155.456, 161.11. MS (ES⁺): m/z=271.21.

Synthesis of 5,5′-(((1,4,9-trimethyl-6-oxo-6H-benzo[c]chromene-3,8-diyl)bis(oxy))bis-(methylene))-bis(furan-2-carbonitrile) (9) In a 5 mL glass vial, under nitrogen, derivative 8 (0.25 mmol) was mixed with potassium carbonate (0.6 mmol) and 1 mL anhydrous DMF and stirred for 15 minutes at 70° C. 5-(chloromethyl)furan-2-carbonitrile (2 mmol) was added and the reaction mixture was further stirred for 12 hours at 70° C. The reaction was subsequently cooled, diluted with EtOAc (10 mL) and washed three times with water (3×10 mL). The organic phase was dried over anhydrous MgSO₄, filtered, and concentrated under reduced pressure. Pure compound was obtained by flash chromatography using an EtOAc/hexane/MeOH gradient (yield 42%). ¹H-NMR δ 2.223 (3H, s), 2.394 (3H, s), 2.865 (3H, s), 5.327 (2H, s), 5.417 (2H, s), 6.931 (2H, m), 7.131 (1H, s), 7.654 (2H, m), 7.878 (1H, s), 8.238 (1H, s). ¹³C-NMR δ 9.071, 17.684, 25.82, 62.479, 62.67, 111.345, 111.525, 112.242, 112.277, 112.593, 112.741, 113.802, 119.899, 125.13, 125.152, 125.614, 125.651, 128.95, 130.367, 134.336, 135.614, 150.469, 155.541, 155.57, 156.625, 156.812, 160.749, 162.921. MS (ES⁺): m/z=481.15.

Synthesis of 3,8-bis((5-(1H-tetrazol-5-yl)furan-2-yl)methoxy)-1,4,9-trimethyl-6H-benzo[c]chromen-6-one (SJ982757, SJ757) In a 5 mL glass vial, under nitrogen, bis-nitrile derivative 6 (0.15 mmol) was mixed with NaN₃ (0.6 mmol), NH₄Cl (0.6 mmol), and anhydrous DMF (1 mL) and stirred overnight at 100° C. The reaction was cooled and iced water (3 mL) added. The reaction mixture was adjusted to pH 11 with NaOH and filtered through celite. The product separated upon acidification to pH 2 as a light-yellow precipitate (yield 41%). ¹H-NMR δ 2.223 (3H, s), 2.39 (3H, s), 2.877 (3H, s), 5.296 (2H, s), 5.372 (2H, s), 6.807 (1H, d, ³J=2.91 Hz), 6.83 (1H, d, ³J=2.91 Hz), 7.015 (2H, b), 7.185 (1H, s), 7.918 (1H, s), 8.232 (1H, s). ¹³C-NMR δ 9.180, 17.853, 25.894, 63.139, 63.307, 107.081, 107.091, 111.207, 111.259, 112.055, 112.860, 113.067, 113.870, 119.793, 128.813, 130.135, 134.175, 135.624, 148.781, 149.066, 150.005, 150.092, 150.455, 154.911, 155.917, 156.043, 160.915. MS (ES+): m/z=567.16.

### NMR Experiments

All NMR experiments were performed at 298 K (25° C.) using a Bruker Avance 600 MHz spectrometer equipped with TCI cryogenic gradient probe. NMR spectra were processed using Bruker Topspin software and analyzed using computer-aided resonance assignment (CARA) software (Keller, R., The Computer Aided Resonance Assignment Tutorial. 2004: CANTINA Verlag.). Two-dimensional (2D)¹H-¹⁵N HSQC NMR experiments were performed using a SampleJet sample changer. Compounds were dissolved at 50 mM each in DMSO-D₆ and were mixed using a Gilson 215 liquid handler with buffer (20 mM Na phosphate, pH 6.5, 200 mM NaCl, 10% ²H₂O, 5 mM DTT-D₁₀) containing ¹⁵N-p27-KID protein (100 μM) to give final compound concentration of 1 mM. Three-dimensional (3D) backbone triple-resonance experiments were performed to establish resonance assignments for the p27 constructs. 2D ¹H-¹⁵N HSQC NMR titrations of optimized compounds SJ749, SJ755, and SJ757, respectively, into ¹⁵N-p27-KID were recorded to determine p27-KID:compound affinity. The following molar ratios of ¹⁵N-p27-KID to compound were used: 1:0, 1:0.5, 1:1, 1:2, 1:4, 1:6, and 1:8. Chemical shift perturbation values were quantified using the equation Δδ=\sqrt{Δδ_{H}²+(0.2Δδ_{N})²}. The statistical significance was based on a threshold defined as the average CSP value plus two times the standard deviation of the mean (Δδₐᵥₑ+2σ). All resonances that exhibited chemical shift perturbations greater than Δδₐᵥₑ+2δ were subsequently plotted against compound concentration and non-linear fitting was performed using GraphPad Prizm 7 software to quantify p27-KID:small molecule interactions and obtain equilibrium dissociation constant (K_{d}) values.

### Analytical Ultracentrifugation (AUC)

Sedimentation velocity experiments were conducted in a ProteomeLab XL-I analytical ultracentrifuge (Beckman Coulter, Indianapolis, Ind.) following standard protocols unless mentioned otherwise (Zhao, H., et al., Curr Protoc Protein Sci, 2013. Chapter 20: p. Unit20 12.). Protein samples (100 μM) in buffer containing 20 mM sodium phosphate pH 6.5, 200 mM NaCl, 5 mM DTT, 2% DMSO without and with compounds (ratio 1:8) were loaded into cell assemblies comprised of double sector charcoal-filled centerpieces with a 12 mm path length and sapphire windows. The density and viscosity of the ultracentrifugation buffer at 20° C. were measured with a DMA 5000M density meter and an AMVn viscometer (both Anton Paar, Graz, Austria), respectively. The cell assemblies, containing identical sample and reference buffer volumes of 390 μL, were placed in a rotor and temperature equilibrated at rest at 20° C. for 2 hours before it was accelerated from 0 to 50,000 rpm. Rayleigh interference optical data were collected at 1-minute intervals for 12 hours. The velocity data were modeled with diffusion-deconvoluted sedimentation coefficient (S) distributions c(S) in SEDFIT, using algebraic noise decomposition and with signal-average frictional ratio and meniscus position refined with non-linear regression (Schuck, P., Biophys J, 2000. 78(3): p. 1606-19.). The S-value was corrected for time, temperature and radial position and finite acceleration of the rotor was accounted for in the evaluation of Lamm equation solutions (Zhao, H., et al., PLoS One, 2015. 10(5): p. e0126420.). Maximum entropy regularization was applied at a confidence level of P−0.68. The partial specific volume of the protein, based on its amino acid composition, was calculated in SEDFIT. All plots were created in GUSSI (Brautigam, C. A., Methods Enzymol, 2015. 562: p. 109-33). A two-dimensional size-shape distribution, c(S, f/f₀) (with one dimension the S-distribution and the other the frictional ratio (f/f₀)) was calculated with an equidistant f/f₀-grid of 0.1 steps that varied from 1 to 3, a linear S-grid from 0.2 to 6 S with 100 S-values, and Tikhonov-Phillips regularization at one standard deviation. The velocity data were transformed to c(M,f/f₀) distributions with M the molecular weight, f/f₀ the frictional ratio, S the sedimentation coefficient and plotted as contour plots. The dotted lines of c(M, f/f₀) indicate constant S values.

### Isothermal Calorimetry (ITC)

ITC experiments were performed using a MicroCal ITC200 calorimeter with p27 variants in the syringe and Cdk2/cyclin A and Cdk2, respectively, in the cell. Prior to each set of experiments, protein samples were extensively dialyzed together against ITC buffer containing 20 mM HEPES, pH 7.5, 300 mM NaCl, 5 mM TCEP. A standard titration consisted of 19 injections of 2-μl of p27 variant (100 μM) into a solution of Cdk2/cyclin A (10 μM) or Cdk2 (10 μM), respectively. The time interval between injections was 180 s. Experiments were performed at 25° C. Thermodynamic parameters were obtained by fitting the raw data using Origin software (OriginLab) according to the manufacturers instructions using a 1:1 binding model to obtain values of the enthalpy of binding (ΔH), Gibbs free energy of binding (ΔG), entropy of binding (ΔS), and stoichiometry factor (N). Experiments were performed in triplicate and mean and standard deviations of the mean values of these parameters are reported.

### Cdk2/Cyclin a Kinase Activity Assays

Cdk2/Cyclin A (100 μM) was mixed with Histone H1 (50 μM; EMD Millipore) and varied amounts of p27 constructs and incubated for 3 hours at 4° C. Subsequently, ATP (50 μM total concentration, of which 10 μCi γ ³²P-ATP (PerkinElmer, Inc.) was added to each reaction and further incubated for 30 minutes at 35° C. Each reaction had a total volume of 20 μL. The sample buffer contained 20 mM HEPES pH 7.3, 25 mM sodium β-glycerolphosphate, 15 mM MgCl₂, 16 mM EGTA, 0.5 mM Na₃VO₄ and 10 mM DTT. The reactions were quenched by addition of SDS-gel loading buffer (7 μL) and then analyzed by SDS-PAGE (10 μL). The gels were dried at 70° C. under vacuum and a phosphoimager (GE Healthcare, Piscataway, N.J.) was used to quantify the ³²P-Histone H1 bands. IC₅₀ values were determined by curve fitting using GraphPad Prizm 7 software. Experiments were performed in triplicate and mean IC₅₀ and standard deviations of the mean values are reported.

### Results

Identification of Improved p27-Binding Small Molecule Through Cheminformatics Analyses and Chemical Synthesis.

### Screening of Commercially-Available Compounds Identified Using Cheminformatics Methods (Termed SAR-by-Catalog)

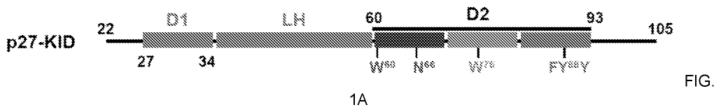

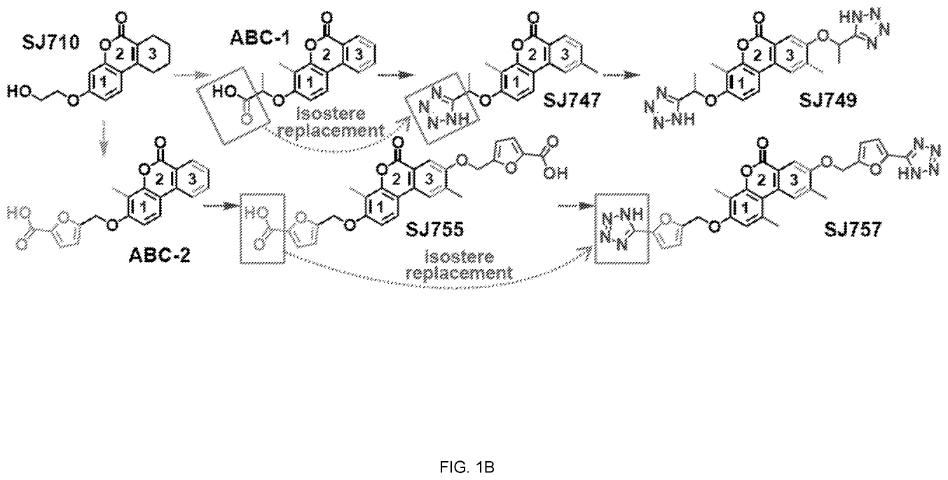

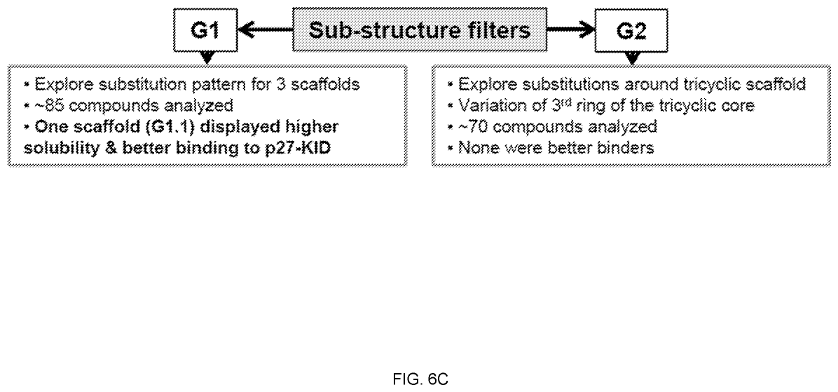

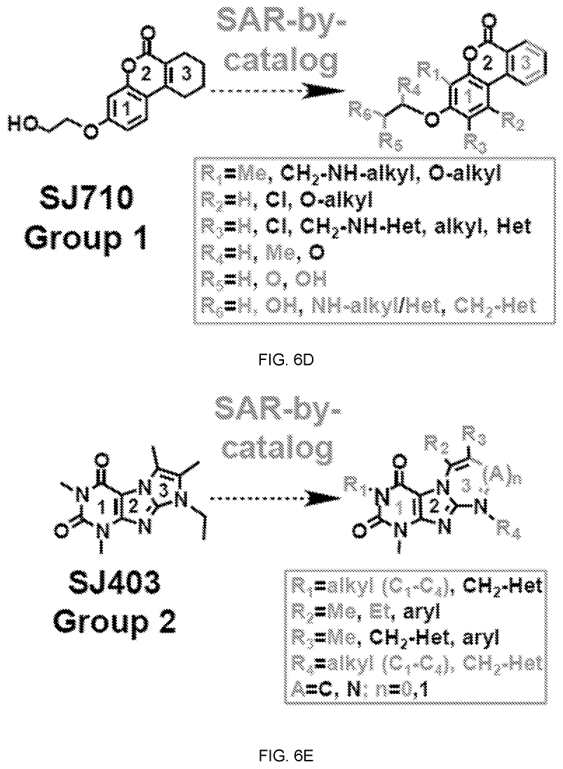

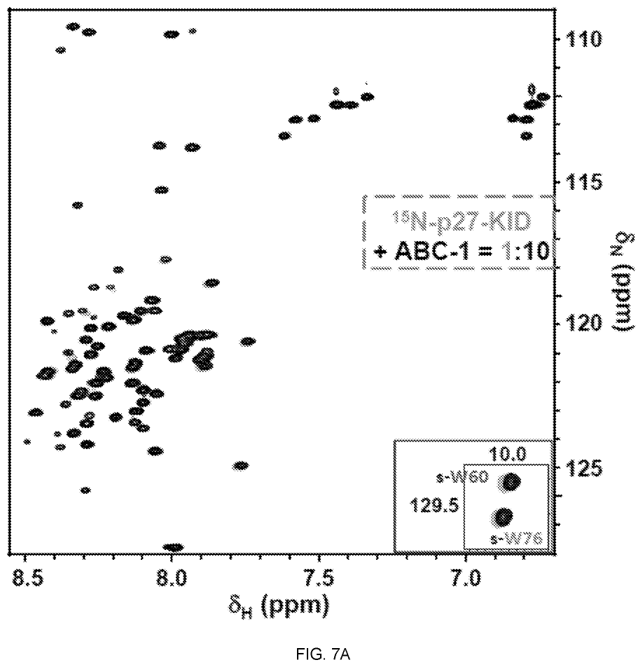

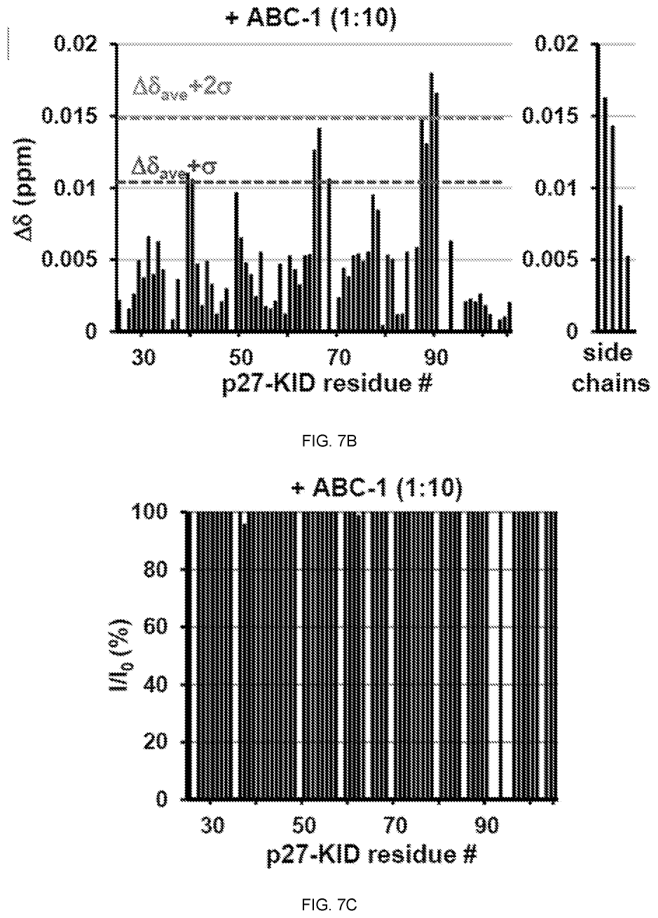

We analyzed our original series of compounds that bind to p27 using sub-structure filters to identify commercially available compounds with similar chemical features (termed SAR-by-catalog, FIGS. 1A-1E). The sub-structure search method identifies analogs that retain the central scaffold of known active compounds but allows for varied substitution patterns. This cheminformatics method was applied to one Group 2 (G2) and three Group 1 (G1) p27-binding compounds from our earlier report (Iconaru, L. I., et al., Sci Rep, 2015. 5: p. 15686) and guided the purchase of ˜160 additional compounds, which were screened for binding to the kinase inhibitory domain (FIG. 1A) of p27 (¹⁵N-labeled p27-KID) using 2D ¹H-¹⁵N HSQC NMR. Most of the newly identified p27-binding compounds (termed “hits”) exhibited patterns of NMR chemical shift perturbations (CSPs) similar to those of prior G1 & G2 compounds; however, analogs of the G1.1 scaffold (e.g., SJ710, see FIG. 1B, FIG. 6A) identified by sub-structure analysis exhibited improved solubility and p27-KID binding (based upon larger and/or more extensive CSPs, data not shown). Purchased compounds (termed “Analog-by-catalog”, or “ABC” compounds) related to the G1.1 scaffold displayed varied Ring 1 substituents and a few included an aromatic Ring 3 (FIG. 6D). Notably, Ring 3 aromaticity slightly enhanced interactions with the “FY₈₈Y” region of p27-KID (FIG. 7A, ABC-1). Screening of additional purchased compounds identified ABC-2 (FIG. 1B), which contains a 2,5-substituted furan heterocycle between the tricyclic core and the carboxylic functional group found in ABC-1. For the G2 scaffold, purchased analogs displayed varied substitutions on both Rings 1 and 3 as well as varied size and heteroatom composition of the 3^{rd} ring of the tricyclic core (FIG. 6E). Most G2 analogs exbibited high aqueous solubility but none caused NMR CSPs of greater magnitude than the parent compound, SJ403.

Chemical Synthesis of p27-Binding Compounds (Termed SAR-by-Synthesis)

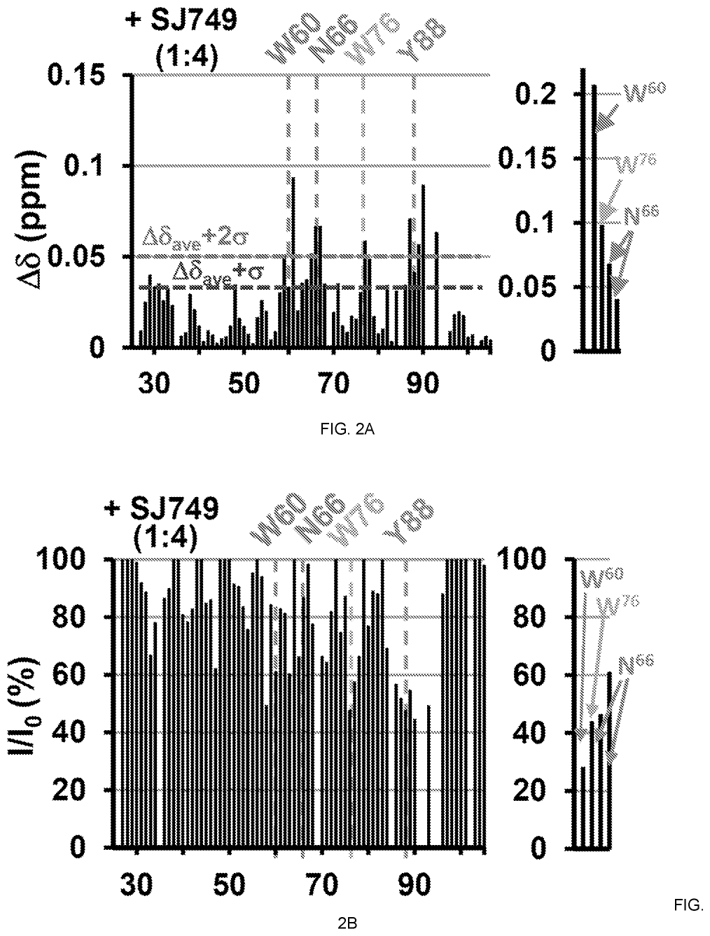

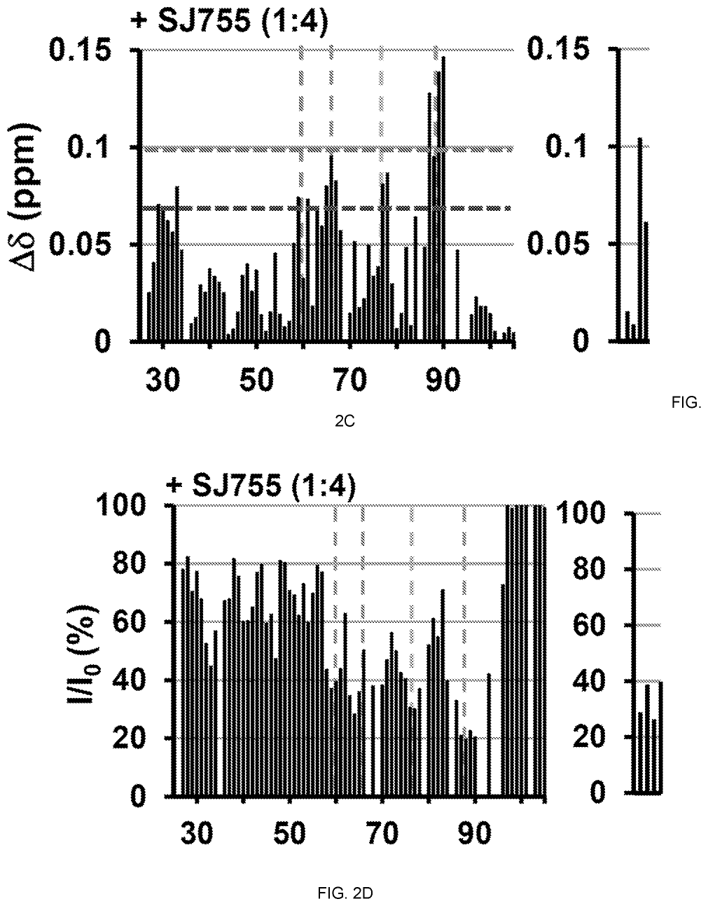

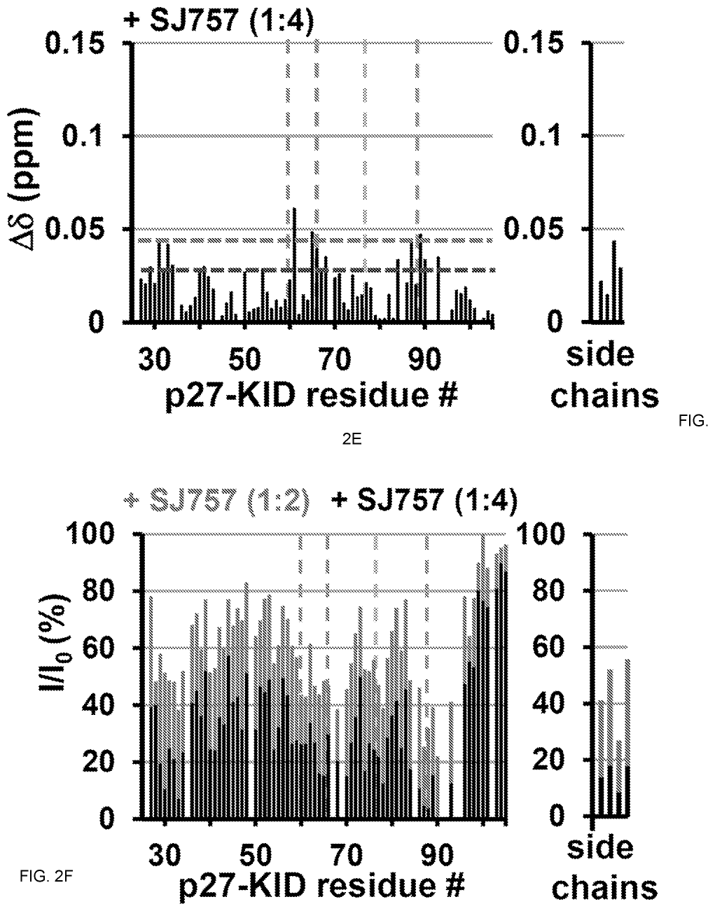

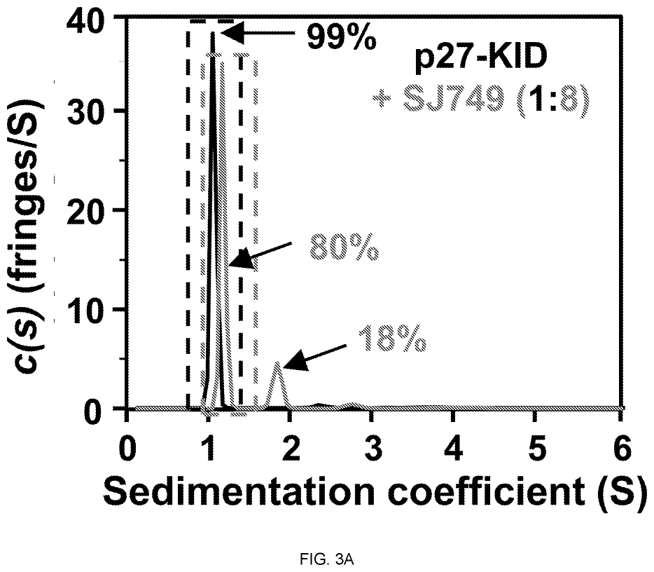

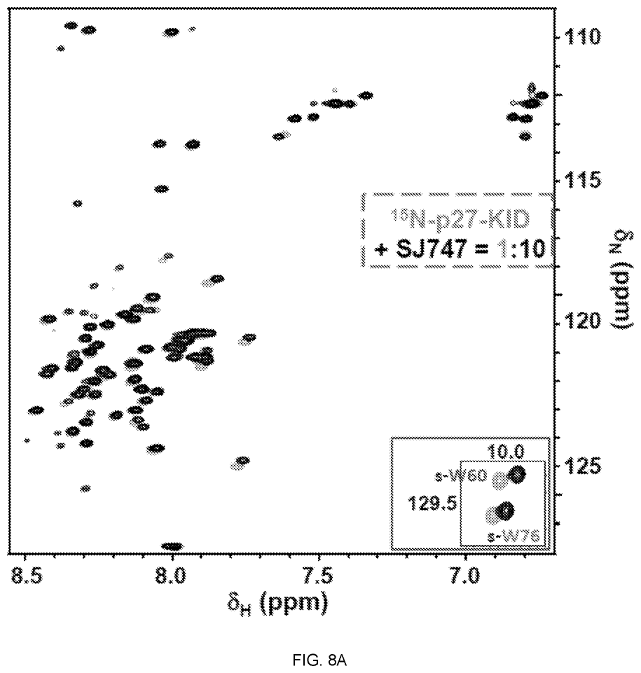

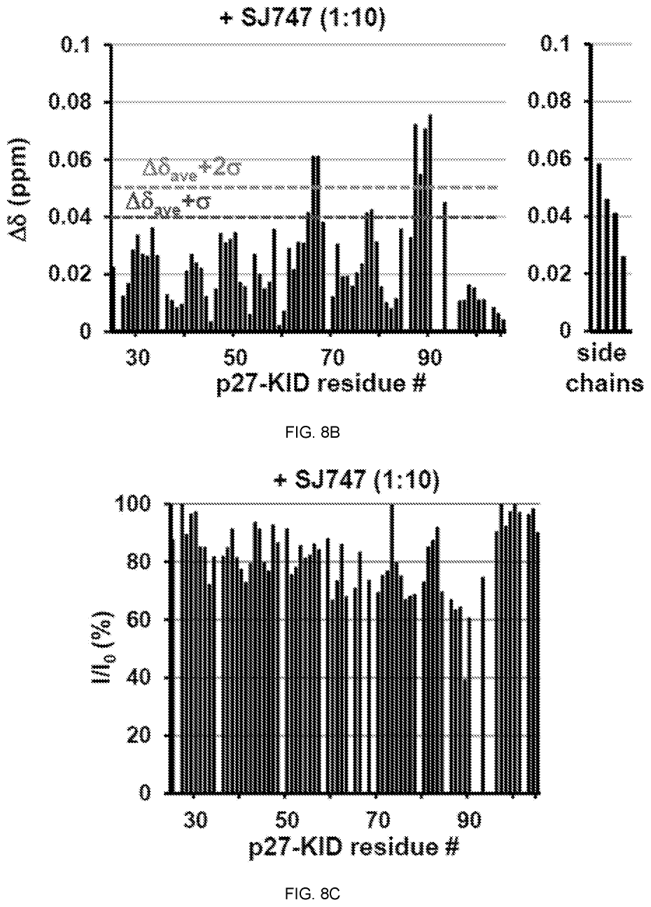

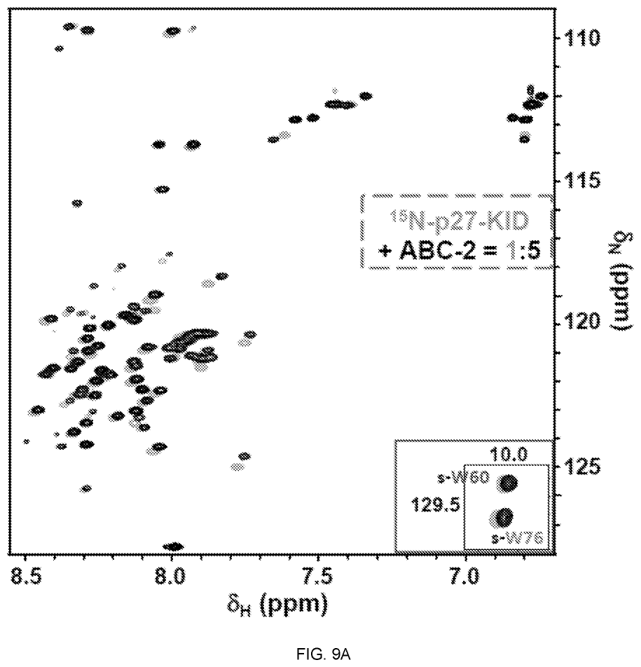

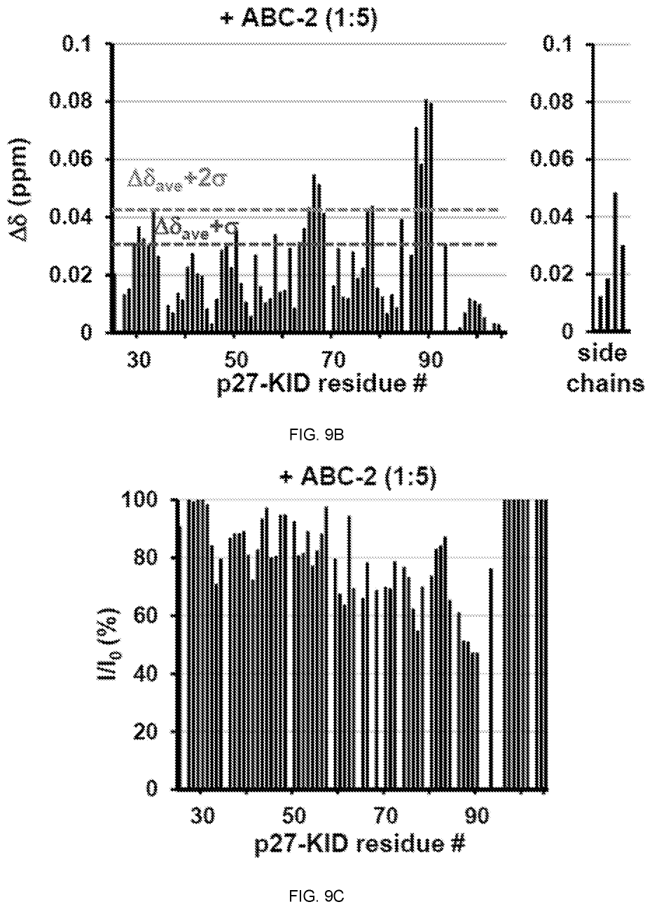

The results of the screening experiments discussed above (summarized in FIGS. 6A-6E), coupled with modifications based upon the concept of bioisostere replacement (Wood, D. J., et al., J Chem Inf Model, 2012. 52(8): p. 2031-43 and references therein), guided derivatization of the G1.1 scaffold through chemical synthesis. From a synthetic perspective, inclusion of an aromatic Ring 3 offers access to diverse analogs due to commercial availability of 4,5-substituted-2-bromo-benzoic acids. Condensation of resorcinols with bromobenzoic acids (Bruggink, A. and A. McKillop, Tetrahedron, 1975. 31(20): p. 2607-2619.) yielded tricyclic intermediates, which served as building blocks for subsequent chemical elaboration (Scheme 1A). Isostere replacement of the —COOH functional group of ABC-1 with a tetrazole moiety (FIG. 1B, SJ747; see Scheme 1B for synthetic scheme) enhanced interactions with several regions within p27 (FIGS. 8A-8C). SJ747 caused NMR CSPs exclusively for amide groups of residues within the D2 sub-domain of p27-KID (p27-D2), but the perturbation patterns were more extensive than for either of the original G1 and G2 scaffolds (compare FIGS. 8A-8C with FIGS. 1-2 from Iconaru, L. I., et al., Discovery of Small Molecules that Inhibit the Disordered Protein, p27(Kip1). Sci Rep, 2015. 5: p. 15686). Analysis of CSP data for compounds with and without the tetrazole moiety indicated that this carboxylic acid isostere interacted with the N66 and W76 residues of p27 (FIGS. 8A-8C). There are two tryptophan residues in p27-KID; therefore, we reasoned that incorporation of a second tetrazole moiety into G1.1 analogs might improve binding to p27. Compound SJ749 (Scheme 2) displays two tetrazole moieties and exhibited enhanced interactions with the W₆₀N₆₁ region and increased affinity for p27-KID (FIG. 2A, vide infra). Further elaboration to include the furan ring of compound ABC-2 afforded the bis(carboxyl) compound, SJ755 (Scheme 2), and the corresponding bis(tetrazole) compound, SJ757 (Scheme 3).

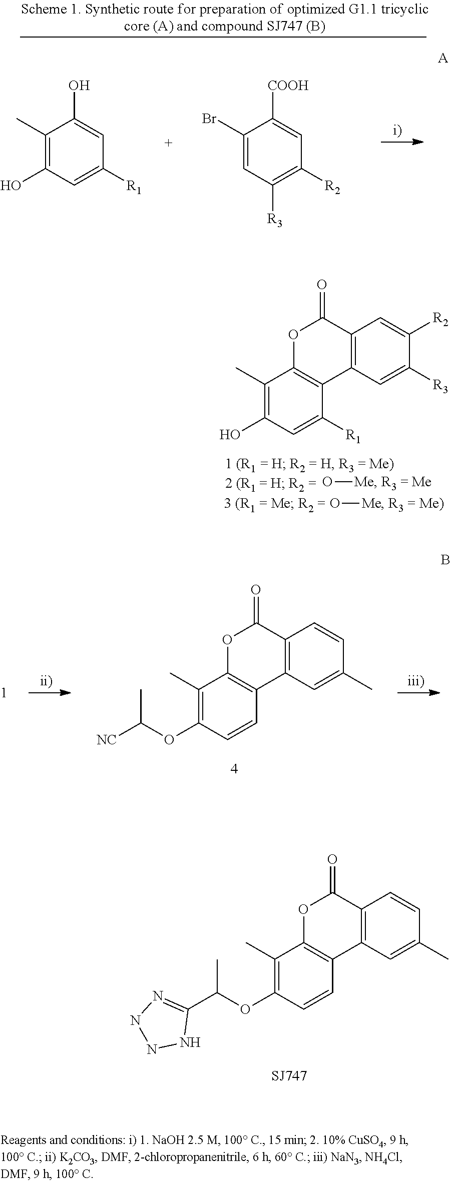

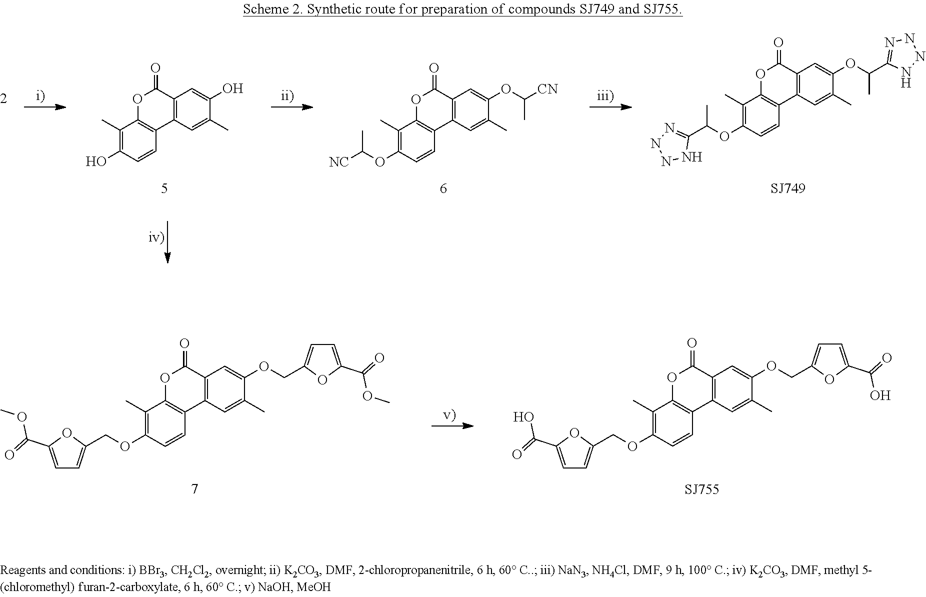

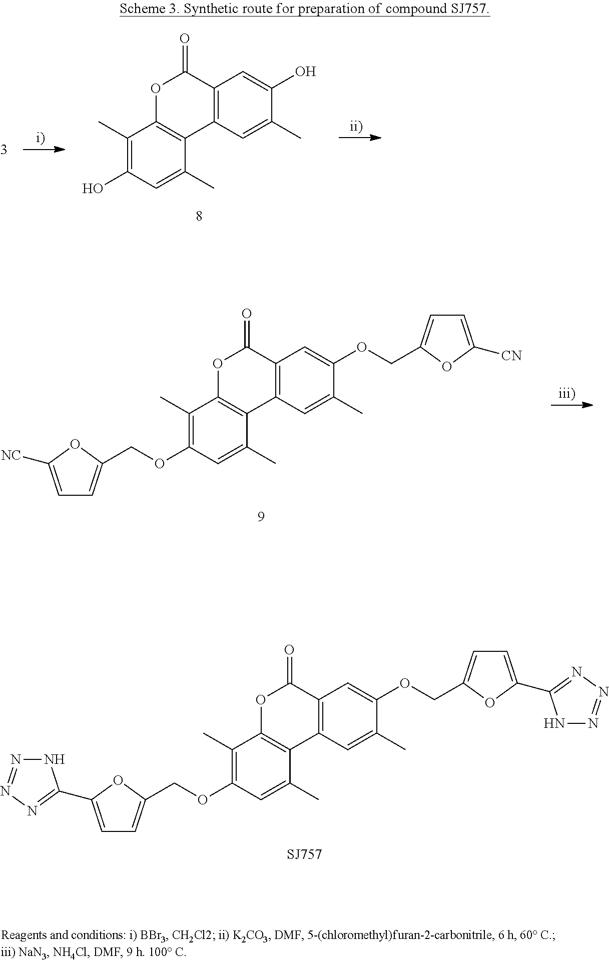

Synthetic Compounds Sequester p27-KID within Soluble Oligomers

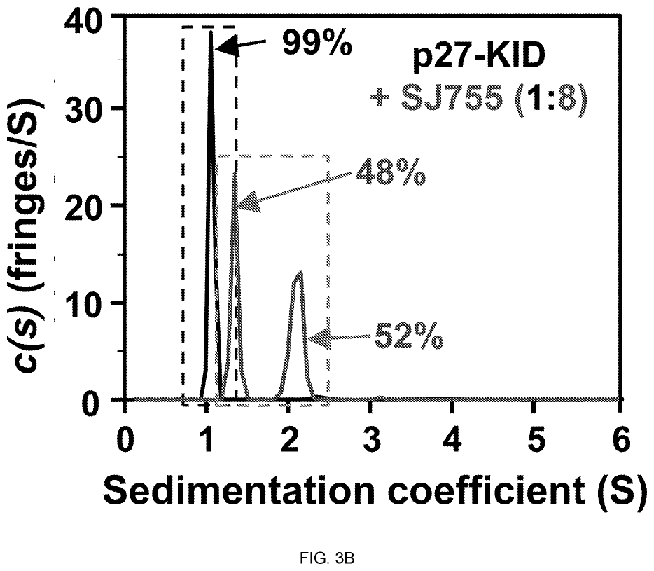

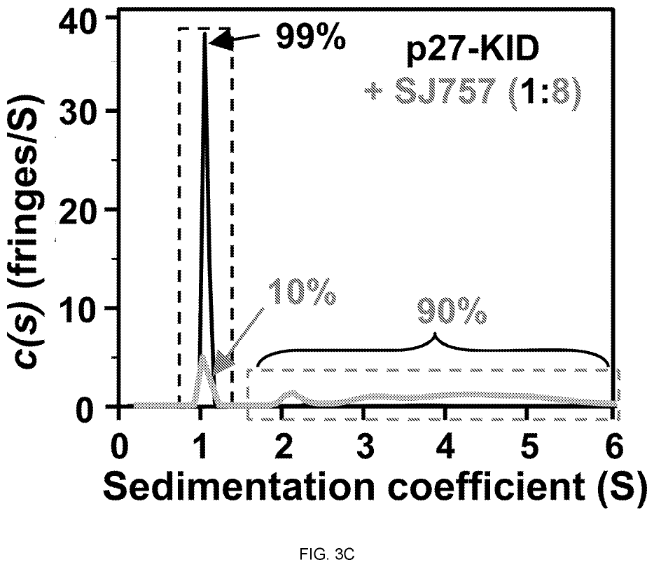

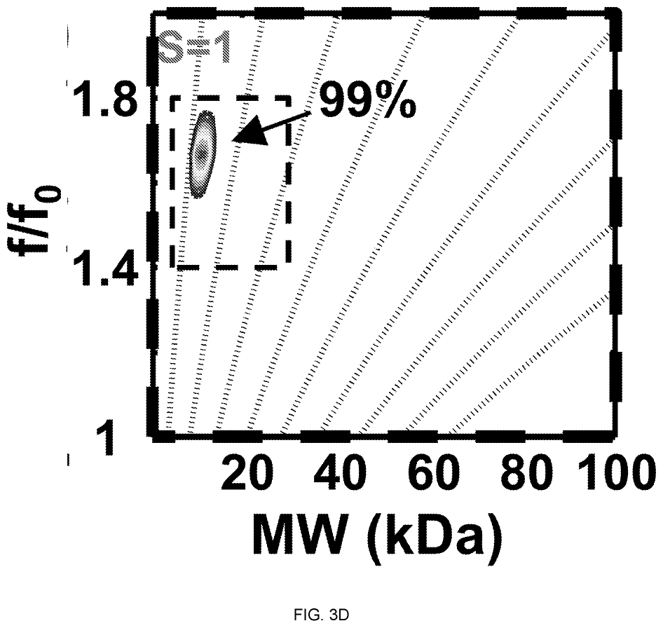

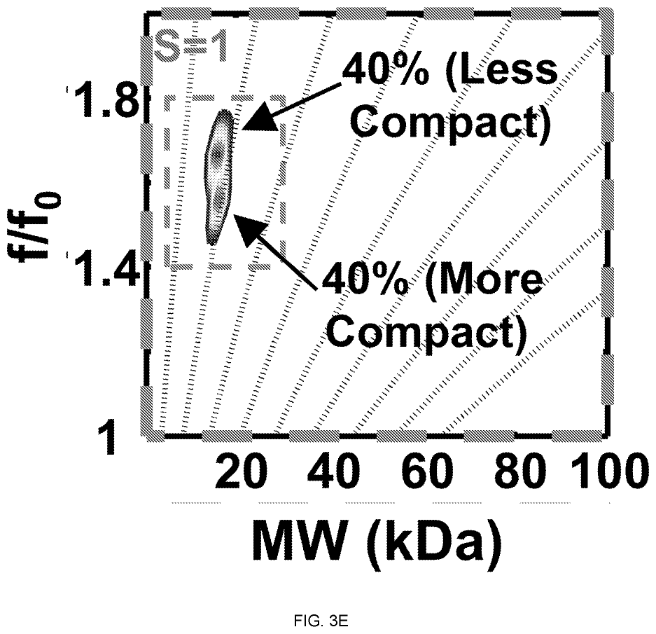

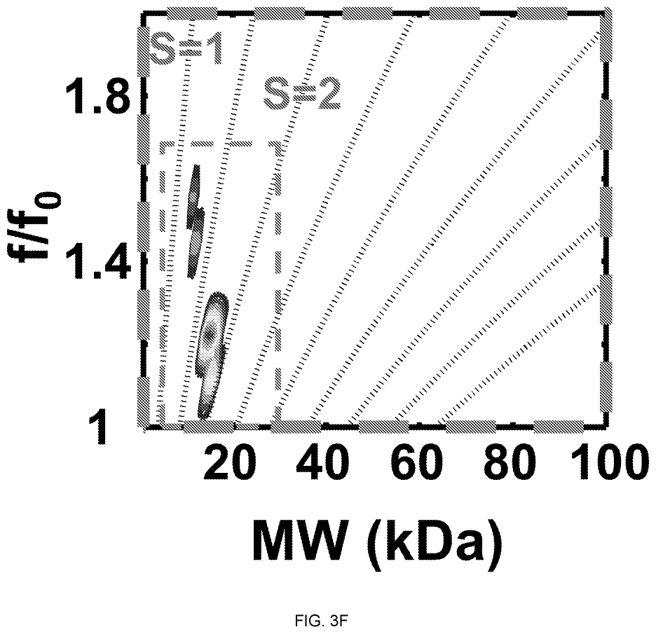

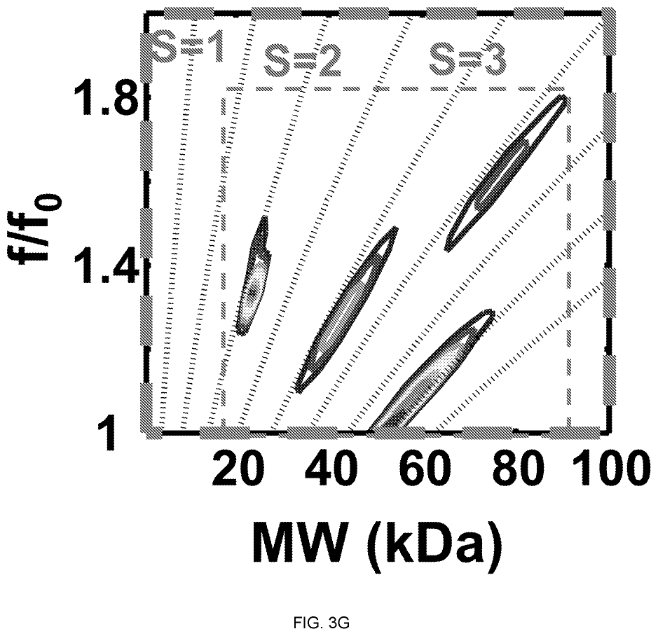

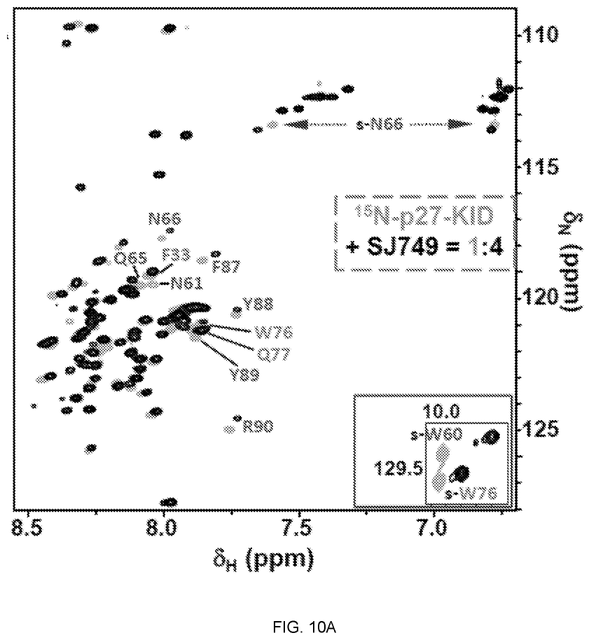

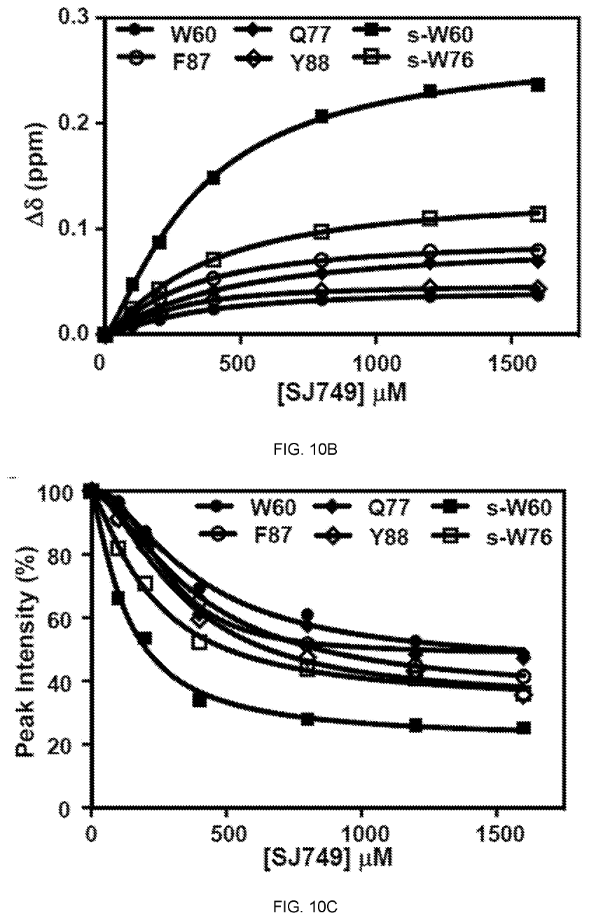

In addition to causing CSPs, binding of SJ749 also caused broadening of resonances of interacting residues in 2D ¹H-¹⁵N HSQC NMR spectra of p27-KID (FIGS. 2A-2B and FIG. 10A). Analysis of CSP and resonance intensities upon titration of SJ749 (FIGS. 10B-10C) yielded K_{d} values of 392±128 μM and 291±76 μM, respectively, for binding to p27-KID (Table 1). These values reflect approximately 10-fold higher affinity binding to p27-KID than the original, parent compound, SJ710. Analysis using sedimentation velocity analytical ultracentrifugation (SV-AUC) showed that isolated p27-KID is monomeric (FIG. 3A, black trace; S=1.04, Table 2) and that, upon addition of excess SJ749, the sedimentation coefficient (S) shifted to a slightly higher value, corresponding to a monomeric p27 species bound to SJ749 (FIG. 3A, gray trace; S=1.16, Table 2). Two-dimensional (2D) analysis of SV-AUC data revealed that SJ749 caused compaction of monomeric p27-KID, with two conformers with different shape factor values (f/f₀) observed (f/f₀=1.66 & 1.55; compare FIGS. 3D-3E). In addition, a peak corresponding to a small population of dimeric p27-KID appeared in the c(S) versus S data with SJ749 (18%, S=1.82; FIG. 3A, gray trace; Table 2) but low intensity prevented 2D data analysis. Thus, SJ749 caused compaction of p27-KID and promoted formation of a minor dimeric species.

TABLE 1
Equilibrium dissociation constant (K_{d}) values obtained from
analysis of 2D ¹H-¹⁵N HSQC NMR spectra recorded as
compounds were titrated into ¹⁵N-p27-KID. K_{d} values were
obtained by fitting a single-site binding model to chemical shift
perturbation (CSP) and relative peak intensity (I/I₀) values.

| Compound | K_{d} (from CSP) | K_{d} (from I/I₀) |
|---|---|---|
| SJ710 | 4.8 ± 1.3 mM | n. d. |
| SJ749 | 392 ± 128 μM | 291 ± 76 μM |
| SJ755 | 338 ± 50 μM | 229 ± 56 μM |
| SJ757 | 140 ± 80 μM | 57 ± 19 μM |

TABLE 2
Summary of Sedimentation Velocity Analytical Ultra-
centrifugation (SV-AUC) data analysis. Results of the velocity
c(s) analysis of samples containing proteins alone (100 μM p27-
KID) and in the presence of synthetic compounds (ratio of 1:8),
respectively. Rayleigh interference optical data were collected.

| | 1D SV-AUC | | 2D c(s, f/f₀) | |
|---|---|---|---|---|
| | s20 | MW | s20 | Frictional |
| Sample | (Svedberg) | (Da) | (Svedberg) | ratio (f/f₀) |
| p27-KID | 1.04 (99%) | 11,270 | 1.04 | 1.67 |
| p27-KID + | 1.16 (80%) | 13,260 | 1.26 (40%) | 1.66 |
| SJ749 | | | 1.45 (40%) | 1.55 |
| | 1.82 (18%) | 26,070 | Not detected | Not detected |
| p27-KID + | 1.37 (48%) | 11,780 | 1.26 (10%) | 1.44 |
| SJ755 | | | 1.43 (5%) | 1.33 |
| | 2.13 (52%) | 22,802 | 1.72 (40%) | 1.22 |
| | | | 1.93 (25%) | 1.12 |
| p27-KID + | 1.04 (10%) | 12,370 | Not detected | Not detected |
| SJ757 | 2.16 (18%) | 36,881 | 2.13 (13%) | 1.35 |
| | 3.18 (53%) | 65,664 | 3.33 (17%) | 1.27 |
| | | | 3.89 (10%) | 1.60 |
| | 4.70 (19%) | 118,300 | 4.80 (4%) | 1.10 |
| p27-KID | 0.97 (99%) | 11,220 | 0.97 | 1.78 |
| W60A- | | | | |
| W76A | | | | |
| p27-KID- | 0.94 (44%) | 10,190 | 1.19 (11%) | 1.75 |
| W60A- | 2.05 (10%) | 32,621 | Not detected | Not detected |
| W76A + | 2.86 (19%) | 53,856 | 3.19 (20%) | 1.40 |
| SJ757 | 4.16 (10%) | 94,428 | | |

TABLE 3
Thermodynamic parameters obtained using
isothermal calorimetry at 25° C.

| | | | | −TΔS |
|---|---|---|---|---|
| | | ΔG | ΔH | (kcal/ |
| Interaction | K_{d} (nM) | (kcal/mol) | (kcal/mol) | mol/deg) |
| Cdk2/cyclin A + p27- | 12.9 ± 0.2 | −10.7 ± 0.1 | −43.6 ± 0.1 | 32.85 ± 0.1 |
| KID | | | | |
| Cdk2/cyclin A + p27- | 16.9 ± 0.6 | −10.6 ± 0.1 | −35.3 ± 0.3 | 24.66 ± 0.3 |
| KID-W60A | | | | |
| Cdk2/cyclin A + p27- | 20.3 ± 2.5 | −10.5 ± 0.1 | −30.3 ± 0.8 | 19.83 ± 0.9 |
| KID-W76A | | | | |
| Cdk2/cyclin A + p27- | 17.0 ± 1.6 | −10.6 ± 0.1 | −24.2 ± 0.1 | 13.6 ± 0.1 |
| KID-W60A-W76A | | | | |
| Cdk2/cyclin A + p27- | 133.6 ± 23.5 | −9.4 ± 0.1 | −24.7 ± 1.8 | 15.3 ± 1.9 |
| D2 | | | | |
| Cdk2/cyclin A + p27- | Binding not | — | — | — |
| D2-W60A | detected | | | |
| Cdk2/cyclin A + p27- | Binding not | — | — | — |
| D2-W76A | detected | | | |
| Cdk2/cyclin A + p27- | Binding not | — | — | — |
| D2-W60A-W76A | detected | | | |
| Cdk2 + p27-KID | 180.8 ± 21.7 | −9.2 ± 0.1 | −23.2 ± 0.1 | 14 ± 0.1 |
| Cdk2 + p27-KID- | Binding not | — | — | — |
| W60A | detected | | | |
| Cdk2 + p27-KID- | Binding not | — | — | — |
| W76A | detected | | | |
| Cdk2 + p27-KID- | Binding not | — | — | — |
| W60A-W76A | detected | | | |
| Cdk2 + p27-D2 | 230.5 ± 67.4 | −9.1 ± 0.2 | −24.5 ± 0.7 | 15.4 ± 0.9 |
| Cdk2 + p27-D2- | Binding not | — | — | — |
| W60A | detected | | | |
| Cdk2 + p27-D2- | Binding not | — | — | — |
| W76A | detected | | | |
| Cdk2 + p27-D2- | Binding not | — | — | — |
| W60A-W76A | detected | | | |

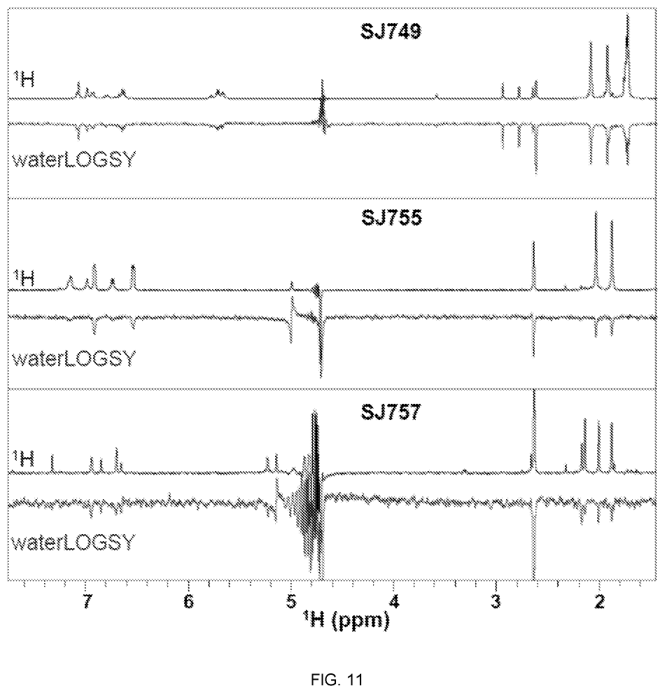

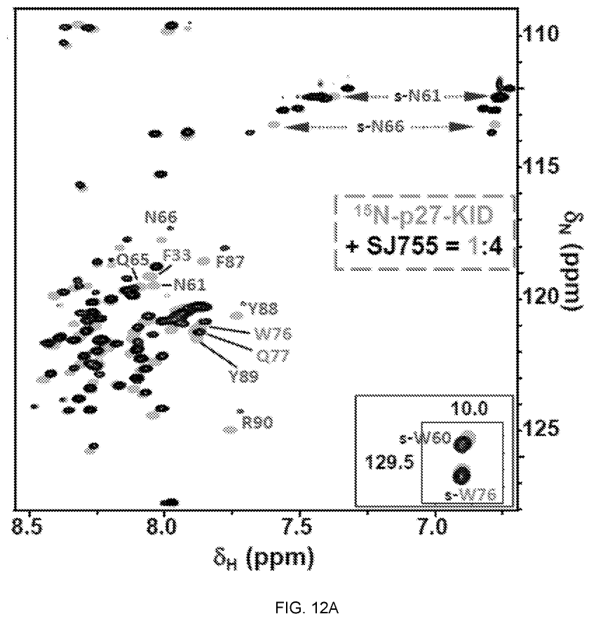

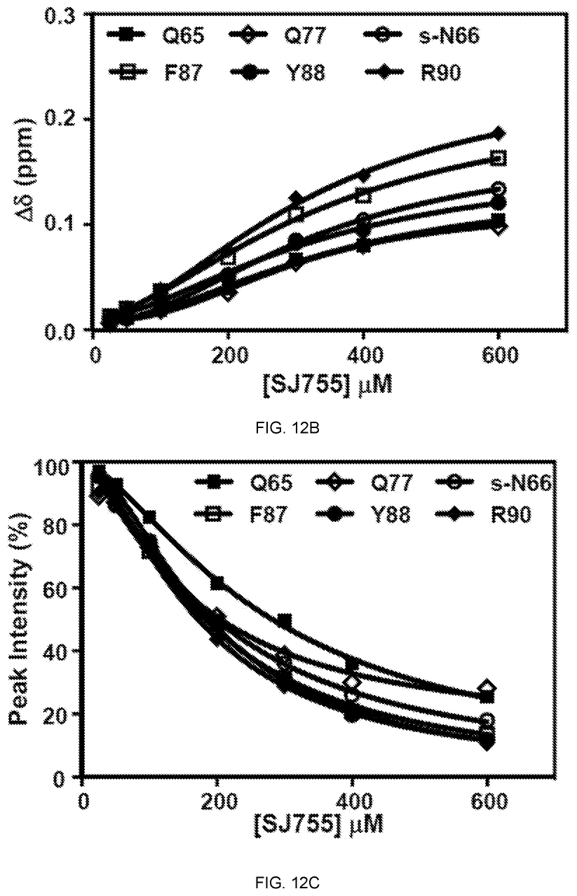

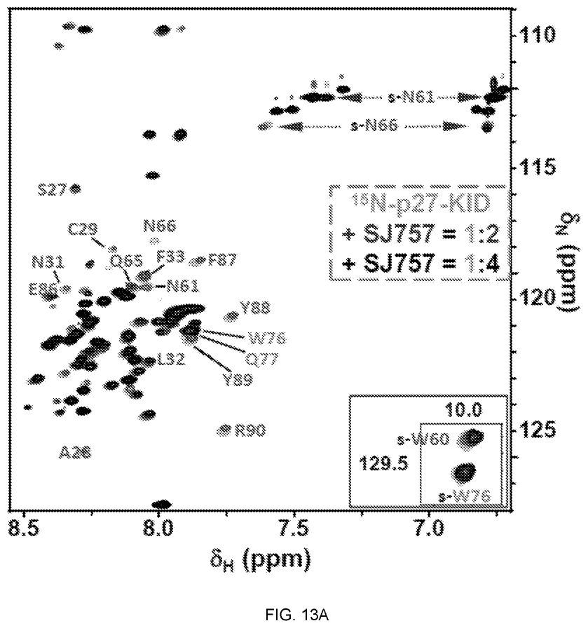

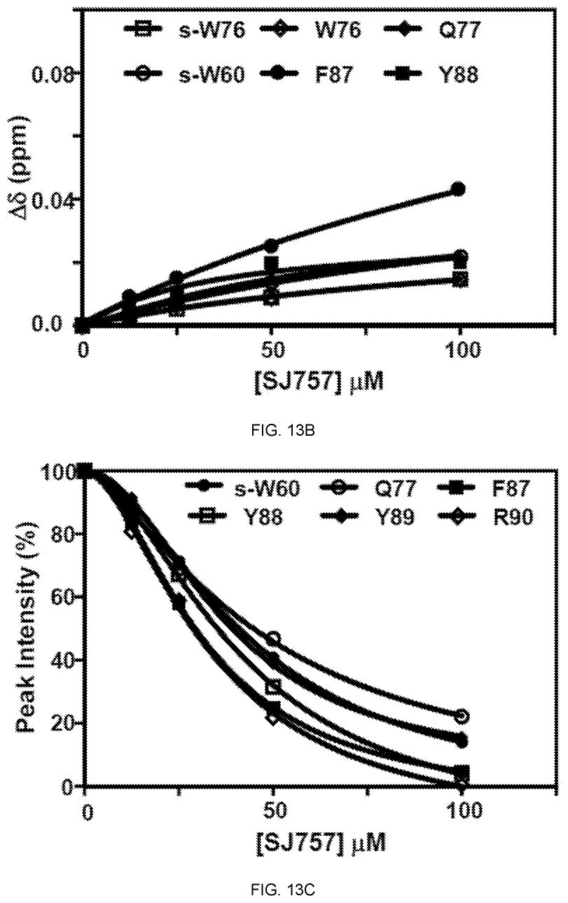

The two compounds with inserted furan rings, SJ755 and SJ757, bound to the D2 region of p27-KID (FIGS. 2C-2F; FIG. 12A, FIG. 13A) with K_{d} values of 338±50 μM and 140±80 μM, respectively (Table 1). Further, SV-AUC analysis showed that the binding of these compounds caused formation of soluble oligomers of p27-KID with a range of molecular sizes (FIG. 3B, FIG. 3C, FIG. 3F & FIG. G). With SJ755, a highly populated, compact dimer formed (52%; FIG. 3B, FIG. 3F; Table 2), while multiple, larger species (with masses ranging from 36,811 Da to 118,300 Da, corresponding to oligomers containing from approximately three to ten p27-KID molecules) were observed with SJ757 (90% of p27-KID molecules; FIG. 3C, FIG. 3G; Table 2). Notably, the furan ring-containing compounds exhibited high aqueous solubility and did not self-aggregate, based on 1D ¹H NMR analyses (FIG. 11). NMR CSP and peak intensity data indicated that SJ749, SJ755, and SJ757 interacted with similar regions of p27-KID, including residues near W60, N66, W76 and Y88; however, due to formation of high molecular weight soluble oligomers, peak broadening was more pronounced for p27-KID in the presence of SJ757, which was also associated with smaller CSP values for the observed resonances (FIG. 2E, FIG. 2F). Also, SJ757 cause broadening of resonances of residues in other regions of p27-KID, including region D1 (residues 27-34), which binds to cyclin A, and region LH, which forms a kinked α-helix linking D1 and D2 (residues 38-59) when bound to Cdk2/cyclin A.

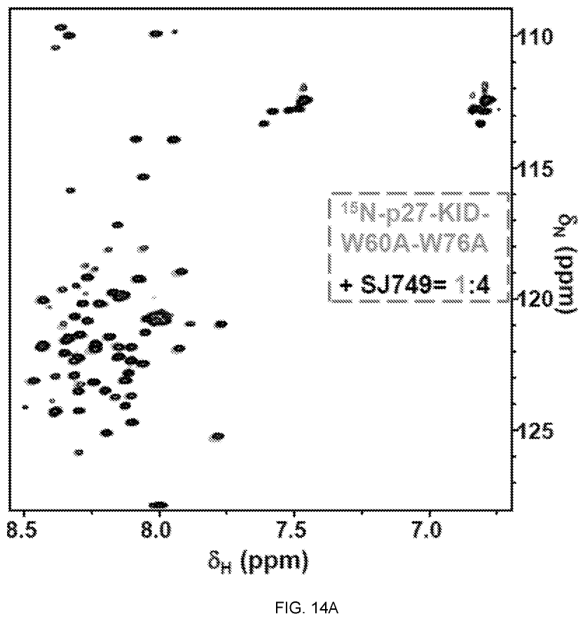

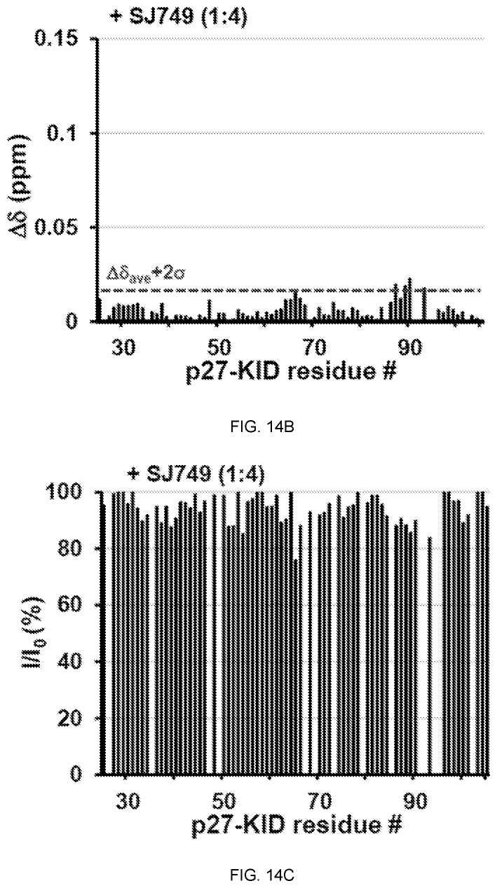

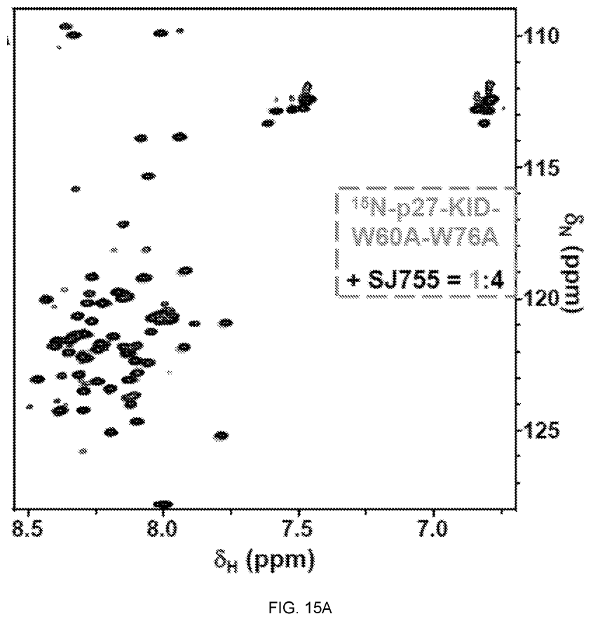

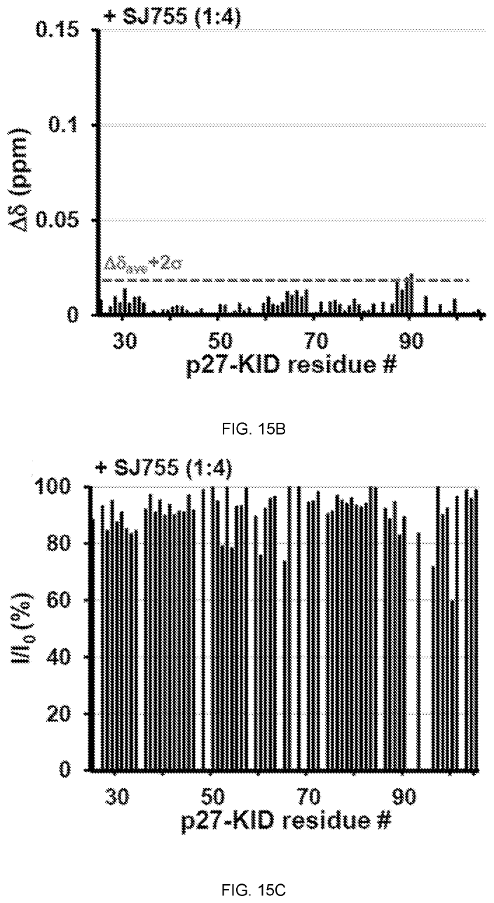

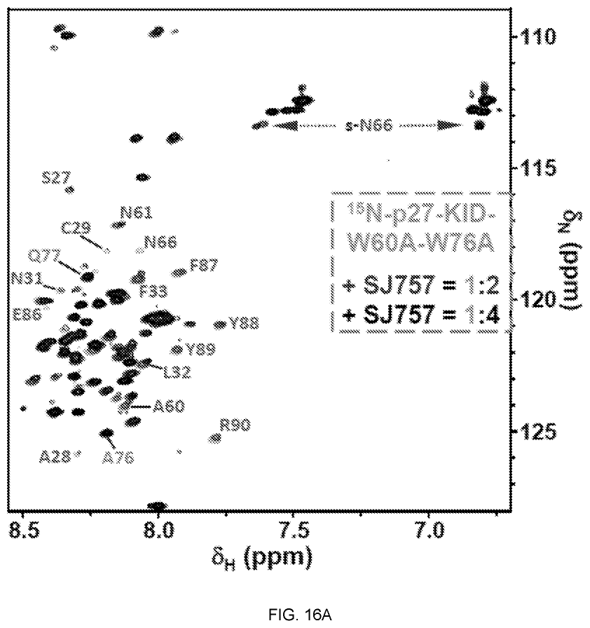

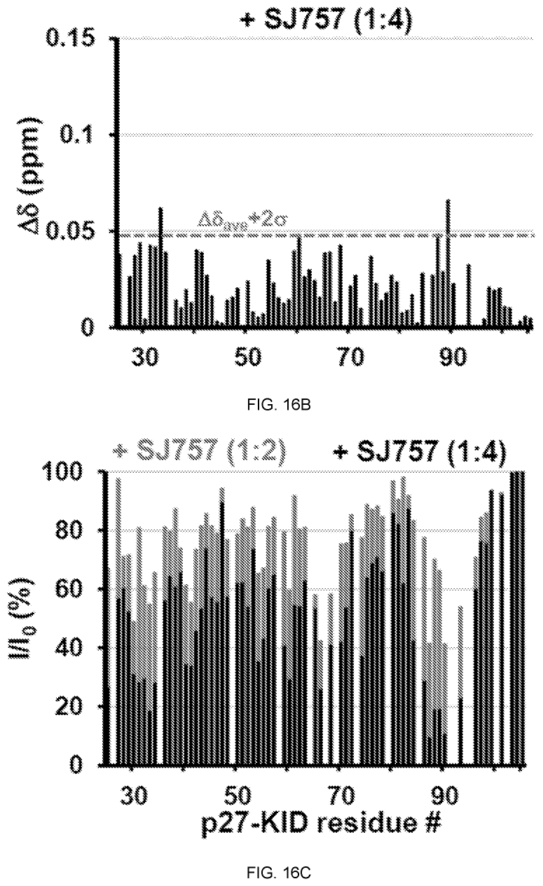

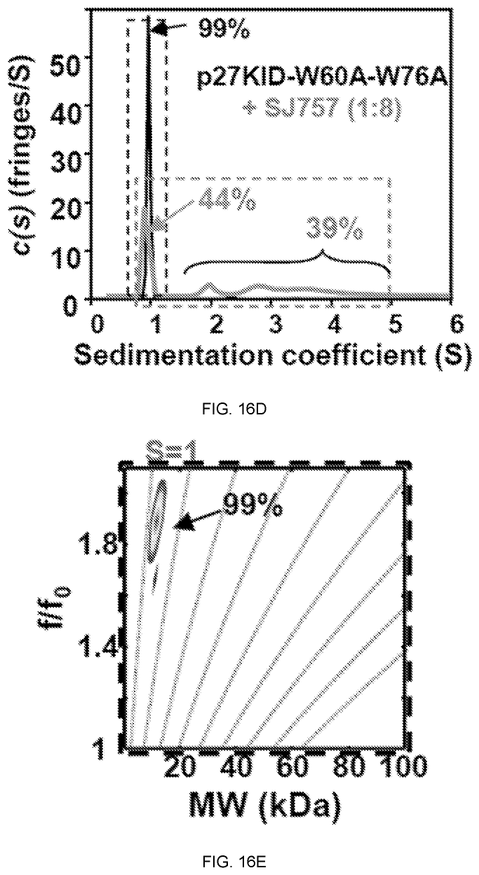

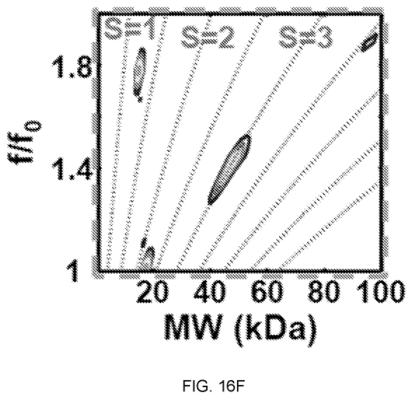

Interestingly, while SJ749, SJ755, and SJ757 significantly perturbed backbone HN resonances for W60 and W76 of p27-KID (CSP values and/or peak intensity values; FIGS. 2A-2C; FIG. 11, FIGS. 12A-12C, FIGS. 13A-13C), only SJ749, which lacks a furan ring, also perturbed chemical shift values of sidechain indole resonances (FIG. 2A, FIG. 2C, FIG. 2E; FIG. 11, FIGS. 12A-12C, FIGS. 13A-13C). These results suggest that the furan rings in SJ755 and SJ757 influence their interactions with the indole sidechains of the two tryptophan residues. Strikingly, mutation of these two residues to alanine (p27-KID-W60A-W76A) abrogated binding to SJ749 and SJ755 (FIGS. 14A-14C, FIGS. 15A-15C) but not to SJ757 (FIGS. 16A-16F). SJ757 bound to p27-KID-W60A-W76A with slightly reduced affinity relative to the wild-type protein but still engaged native residues within the D2 region as well as the D1 and LH regions. These results indicate that SJ757, by virtue of its more elaborate chemical structure, engages residues within multiple regions of p27-KID, enabling sequestration of multiple p27-KID molecules within soluble oligomers.

### Tryptophan Residues Improve for p27 Cdk2 Inhibitory Function

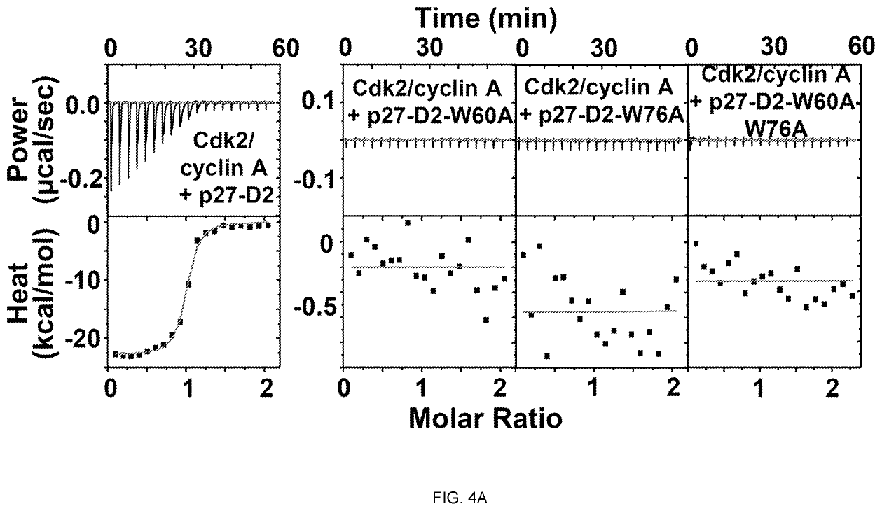

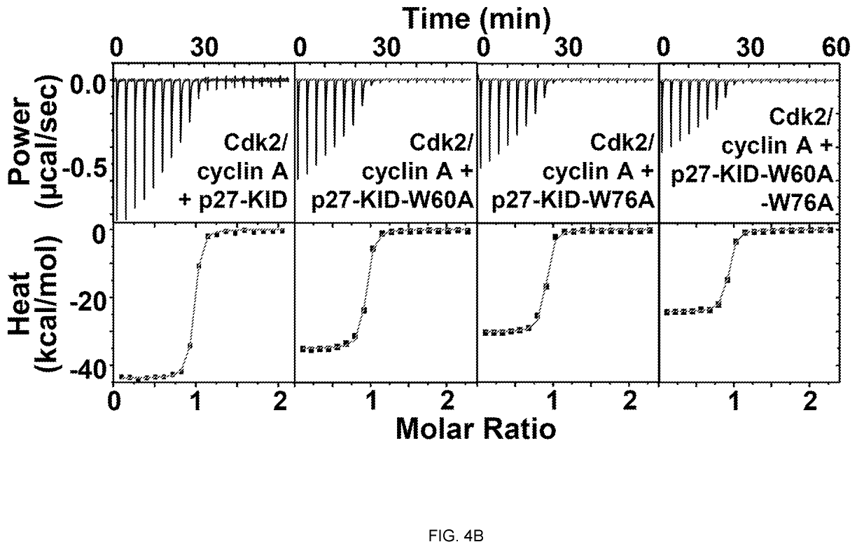

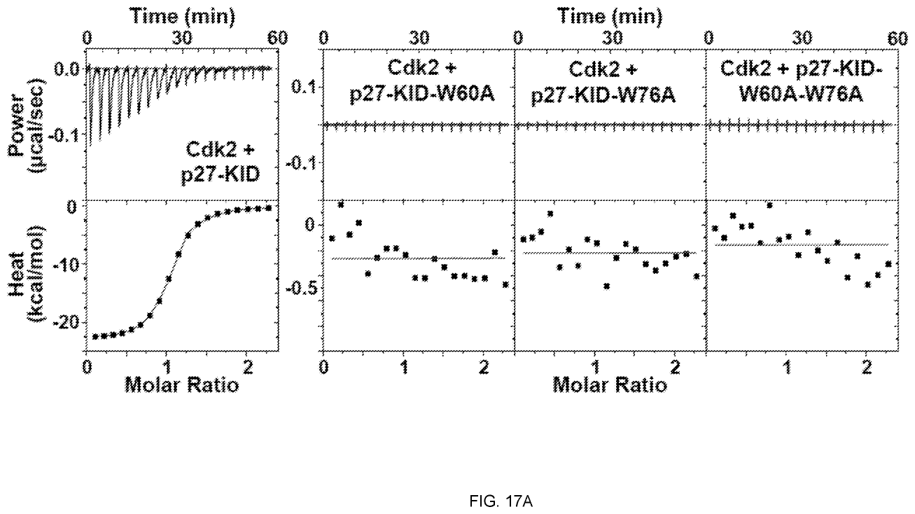

The p27 binding compounds reported herein all interact with the two tryptophan residues within p27-KID. Not wishing to be bound by any particular theory, it is believed that when p27 is bound to Cdk2/cyclin A, the sidechains of these residues may be protected from solvent by packing against the surface of Cdk2, suggesting that they may be important contributors to the Gibbs free energy of binding (AG). This theory was tested by using isothermal titration calorimetry (ITC) to monitor the binding of p27-KID and p27-D2 in which W60 or W76, or both, were mutated to alanine (A), to Cdk2 and the Cdk2/cyclin A complex. The W to A mutations within the p27-D2 construct, which lacks the D1 region that binds tightly to cyclin A, abrogated binding to Cdk2 (FIG. 17B) and Cdk2/cyclin A (FIG. 4A; Table 3), demonstrating that the two tryptophan residues are important contributors to the AG of binding. In the context of p27-KID, the mutations abrogated binding to Cdk2 (FIG. 17A) and caused a reduction of the values of the enthalpy of binding (AH) to Cdk2/cyclin A (FIG. 4B, Table 3), consistent with reduced binding of the mutated regions of p27-KID to Cdk2 within the Cdk2/cyclin A complex. With Cdk2/cyclin A, the AG of binding values for the p27-KID constructs were very similar due to binding of the native D1 region to cyclin A within the Cdk2/cyclin A complex (Table 3).

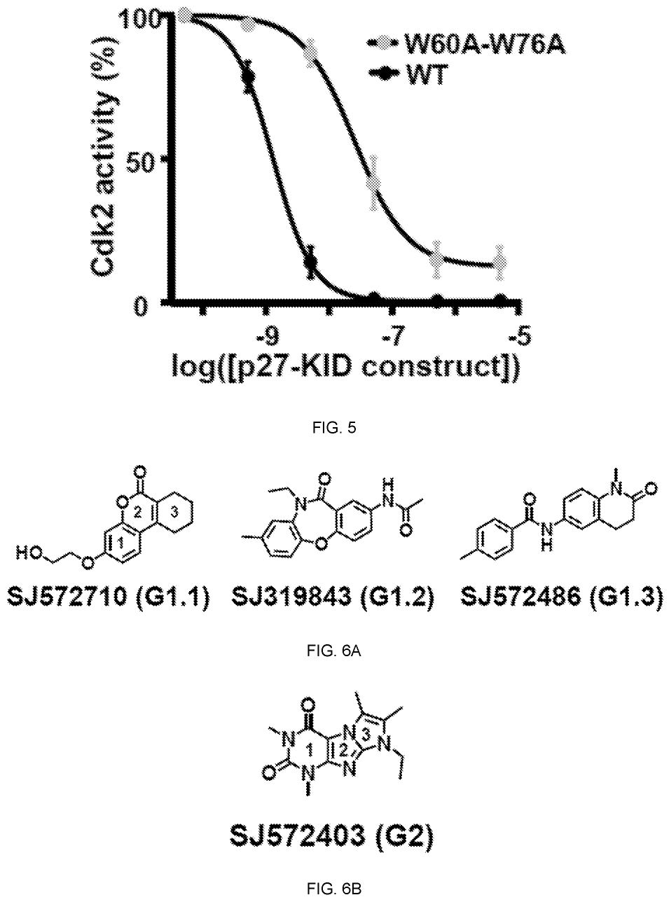

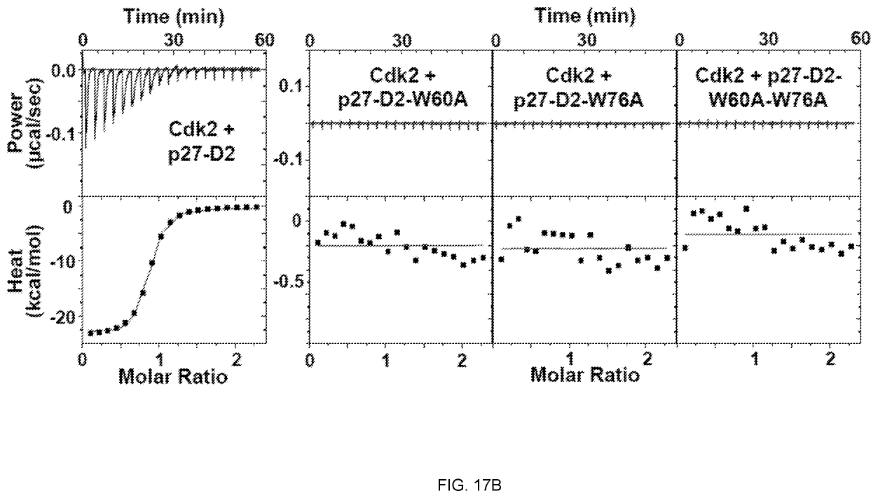

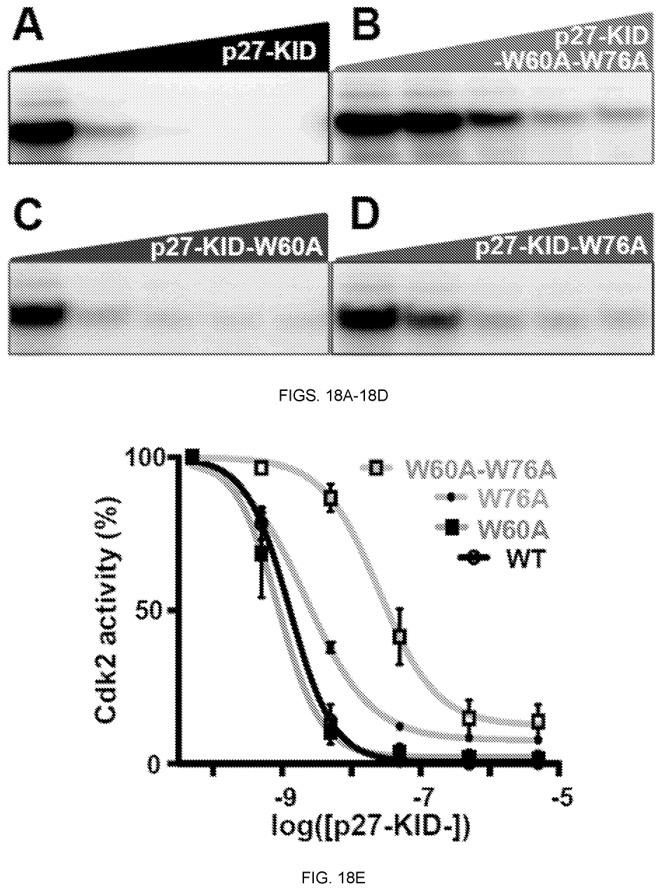

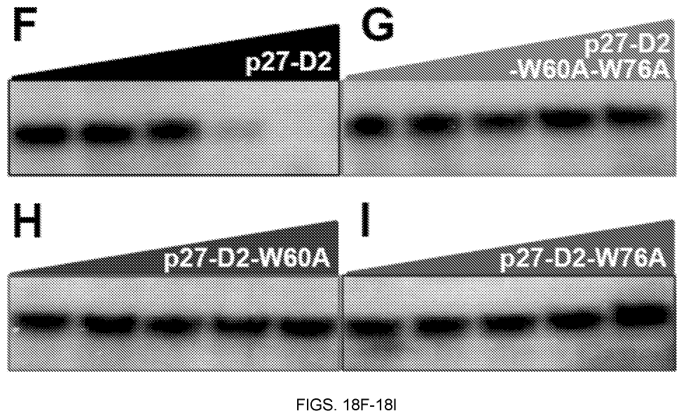

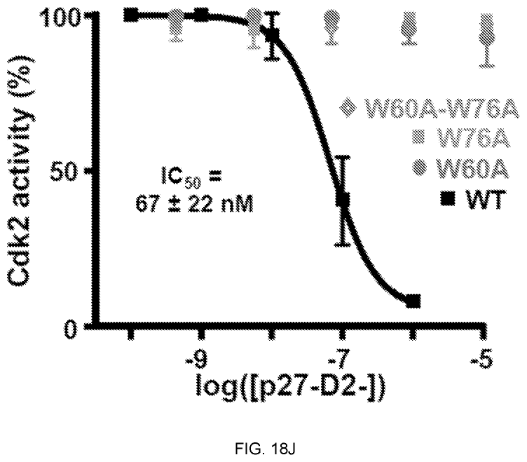

The effect of the W to A mutations in p27-KID on inhibition of the kinase activity of Cdk2/cyclin A toward the substrate Histone H1 (HH1) was investigated. Wild-type p27-KID is a potent inhibitor of Cdk2/cyclin A, with an IC₅₀ value of 1.9±0.3 nM in the current experiments (FIG. 5, FIGS. 18A-18J, Table 4), while p27-D2, which binds only to Cdk2 within the Cdk2/cyclin A complex, exhibited an IC₅₀ value of 67±22 nM (FIGS. 18A-18J). As expected, based upon our ITC results, mutation of individual or both tryptophan residues in p27-D2 abrogated inhibitory activity (FIGS. 18F-18J). However, in the context of p27-KID, the two individual W to A mutations only slightly affected Cdk2 inhibitory activity (IC₅₀ values of 1.7±0.3 nM and 4.3±1.0 nM for the W60A and W76A mutants, respectively; Table 4), while the dual W to A mutant exhibited an IC₅₀ value of 36±8 nM but, even at saturating concentrations, was unable to fully inhibit Cdk2/cyclin A (FIG. 5, FIGS. 18A-18J). The results with the p27-KID mutants suggest that, despite disruption of interactions with Cdk2 due to the individual W to A mutations, Y88 at the C-terminal end of the KID is able to bind within that ATP binding pocket of Cdk2, while the D1 region is bound to cyclin A, and inhibit its catalytic activity. Mutation of both tryptophan residues disrupts binding to Cdk2 further, apparently limiting access of Y88 to Cdk2's active site, increasing the IC₅₀ value and preventing full Cdk2 inhibition (FIG. 5, FIGS. 18A-18J, Table 4). Together, the ITC and Cdk2 inhibition assay results demonstrate that the two tryptophan residues within the D2 region of p27-KID are major contributors to the thermodynamics of binding to Cdk2 within the Cdk2/cyclin A complex and are important for full inhibition of Cdk2 activity.

TABLE 4
Half maximal Cdk2/cyclin A inhibitory concentration (IC₅₀)
values of p27-KID variants using Histone H1 as substrate.

| p27-KID- | IC₅₀ (nM) |
|---|---|
| WT | 1.9 ± 0.3 |
| W60A | 1.7 ± 0.3 |
| W76A | 4.3 ± 1 |
| W60A-W76A | 36 ± 8 |

### Discussion

Our previous NMR-based fragment screening efforts identified aromatic heterocycles that bound weakly but specifically to dynamic clusters of aromatic residues within the D2 region of p27. Through further screening of compounds with similar aromatic heterocyclic core structures but with different ring substituents, compounds (ABC-1 & ABC-2; FIG. 1B) were observed that bound with higher affinity and engaged a larger number residues within p27-KID. Not wishing to be limited by any particular theory, it is believed that duplication of the substituents associated with enhanced binding on the aromatic heterocyclic core might further improve binding. In addition, it is possible that replacement of the carbocylic acid moiety of ABC-1 and ABC-2 with an isosteric tetrazole moiety would enhance binding to p27-KID. The compounds, SJ749, SJ755 and SJ757 were synthesized (FIG. 1B), each with a common aromatic heterocyclic core substituted with two phenyl ether moieties rich in H-bond donors and acceptors. The aromatic heterocyclic core of these compounds preserved interactions with aromatic residues in p27-KID (near residues W60, W76 and Y88) and these were enhanced and extended to also include residues near N66 through introduction of the two tetrazole moieties in SJ749 (FIGS. 2A-2B). Introduction of two carboxy-furan moieties in SJ755 (FIG. 1B) enhanced interactions further (FIGS. 2C-2D). Interestingly, the p27-KID binding enhancements associated with these two types of aromatic heterocyclic core substituents were also associated with compound-dependent formation of soluble dimers of p27-KID (FIGS. 3A-3G; Table 2). This property, compound binding-dependent formation of soluble protein oligomers, was further consolidated in the compound, SJ757, in which the terminal carboxy moiety of SJ755 was replaced with an isosteric tetrazole moiety. Remarkably, this compound sequestered 90% of p27-KID molecules within an array of soluble oligomers comprised of between three and approximately ten p27-KID molecules and an indeterminate number of compound molecules. Not wishing to be limited by any particular explanation, it is possible that these chemically multivalent compounds interact with dynamic clusters of aromatic amino acids in different p27-KID molecules, causing formation of soluble dimers and, with SJ757, higher order soluble oligomers. Because p27-KID dynamically fluctuates between multiple conformations involving clusters of different aromatic amino acids, the two tetrazole-furan moieties of SJ757 may weakly and non-covalently cross-link multiple protein molecules. The large size of SJ757 requires that its multivalency for binding to aromatic amino acids be fulfilled by multiple p27-KID molecules.

p27-KID folds upon binding to Cdk2/cyclin A, with the so-called “RxL” motif within the D1 region binding to a conserved pocket on the surface of cyclin A and the D2 region adopting extensive secondary structure in the course of forming an extensive interface with Cdk2, ultimately positioning Y88 in the ATP binding pocket for kinase inhibition. Eight aromatic residues contribute to the interface between the D2 region of p27-KID and Cdk2, and most of these are the residues engaged by the synthetic compounds identified herein. While, in the absence of Cdk2/cyclin A, these aromatic residues form disordered, constantly fluctuating clusters, they are poised to adopt specific, ordered conformations on the surface of Cdk2. Some of the aromatic residues within the D2 region of p27-KID are essential for interactions with Cdk2 (e.g., W60 and W76; FIGS. 4A-4B, FIG. 5, FIGS. 17A-17B, FIGS. 18A-18J; Table 3 and Table 4) and these are key mediators of interactions with the compounds reported herein (FIGS. 14A-14C, FIGS. 15A-15C, FIGS. 16A-16F). The small molecule-dependent sequestration through soluble oligomerization can provide a general approach for targeting IDPs that experience folding upon binding to their functional partners. The residues within disordered protein regions that participate in specific partner recognition and folding upon binding can, in principle, be leveraged for recognition by chemical moieties within chemical compounds. The high enrichment of aromatic residues within the D2 region of p27-KID is exceptional relative to the usual amino acid compositional bias of disordered protein regions; however, many IDPs display short linear motifs (SLiMs) with conserved sequences that mediate specific folding upon partner binding and these SLiMs often contain amino acids not typically associated with disorder. For example, the N-terminal transactivation domain of p53 contains a SLIM with conserved aromatic and hydrophobic residues that mediates binding to Mdm2. Further, many viral proteins contain multiple, conserved SLiMs and other, longer interaction regions, that could possibly be targeted for sequestration by chemical compounds with binding features like those reported herein for binding p27-KID. Our experimental strategy, which involves NMR-based fragment screening, cheminformatics analysis, molecular elaboration through chemical synthesis and detailed biophysical characterization of protein:compound interactions, is readily adaptable to other disease-associated IDPs. The top compound, SJ757, binds p27-KID with a K_{d} values of 140±80 μM and 57±19 μM from NMR CSP and I/I₀ data, respectively (Table 1). Investment of much more extensive chemical synthesis resources could more thoroughly explore chemical space and potentially increase affinity for p27-KID further, providing opportunities for modulating p27 function in cells. The mechanism of sequestration through soluble oligomerization presented herein differs from the entropy-driven small molecule:disordered protein interaction mechanism discussed by Vendruscolo and co-workers and Liu and co-workers, and thus provides an additional strategy for consideration when seeking to therapeutically intervene in human diseases involving disordered proteins.

It should be emphasized that the above-described embodiments of the present disclosure are merely possible examples of implementations and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

1-20. (canceled)

21. A compound or a pharmaceutically acceptable salt thereof, wherein the compound has a structure according to Formula II 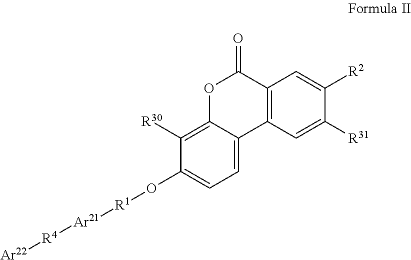
where each occurrence of R³⁰ and R³¹ is independently a hydrogen, a halo, a cyano, a hydroxyl, —NH₂, a C₁-C₃ alkyl, a C₁-C₃ haloalkyl, a C₁-C₃ alkoxy, or a C₁-C₃ haloalkoxy;
where R² is a hydrogen, a halo, a cyano, a hydroxyl, —NH₂, a C₁-C₃ alkyl, a C₁-C₃ haloalkyl, a C₁-C₃ alkoxy, a C₁-C₃ haloalkoxy, or —O—R¹—Ar²¹—Ar²²;
where each occurrence of R¹ and R⁴ is independently a linear or branched chain, substituted or unsubstituted C₁-C₇ alkyl linker;
where each occurrence of Ar²¹ is independently a bond or selected from the group 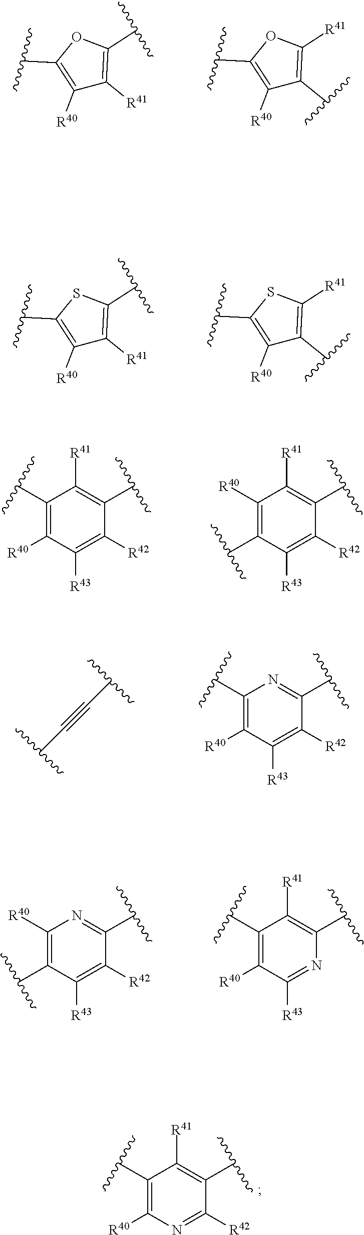
where each occurrence of R⁴⁰, R⁴¹, R⁴², and R⁴³ is independently a hydrogen; a halo, a cyano; a hydroxyl, —NH₂, a C₁-C₃ alkyl; a C₁-C₃ haloalkyl; a C₁-C₃ alkoxy, or a C₁-C₃ haloalkoxy; and
where each occurrence of Ar²² is independently selected from the group 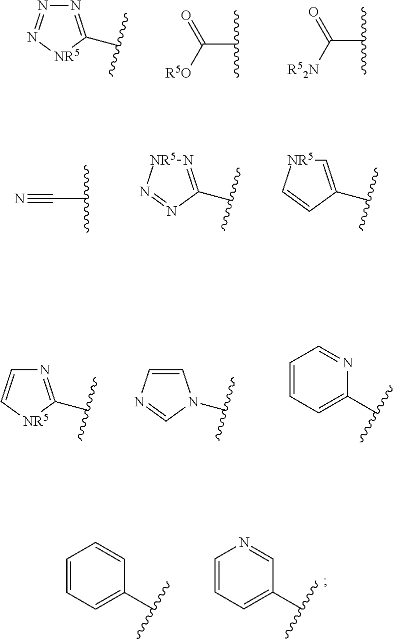
where each occurrence of R⁵ is independently hydrogen, a C₁-C₃ alkyl, or a C₁-C₃ alkoxy.

22-23. (canceled)

24. The compound or pharmaceutically acceptable salt according to claim 21, wherein each occurrence of R¹ is a linear or branched, C₁-C₃ alkyl linker.

25-26. (canceled)

27. The compound or pharmaceutically acceptable salt according to claim 21, wherein each occurrence of R⁴ is a linear or branched, C₁-C₃ alkyl linker.

28-29. (canceled)

30. The compound or pharmaceutically acceptable salt according to claim 21, wherein R³⁰ and R³¹ are methyl.

31-40. (canceled)

41. The compound or pharmaceutically acceptable salt according to claim 21, wherein R² is hydrogen.

42-49. (canceled)

50. The compound or pharmaceutically acceptable salt according to claim 21, wherein R² is —O—R¹—Ar²¹—Ar²².

51. The compound or pharmaceutically acceptable salt according to claim 21, wherein each occurrence of R⁴⁰, R⁴¹, R⁴², and R⁴³ is hydrogen or hydroxyl.

52. The compound or pharmaceutically acceptable salt according to claim 21, wherein at least one occurrence of R⁴⁰, R⁴¹, R⁴² and R⁴³ is methyl and the remaining occurrences are either hydrogen or hydroxyl.

53. The compound or pharmaceutically acceptable salt according to claim 21, wherein R⁵ is hydrogen.

54. (canceled)

55. The compound or pharmaceutically acceptable salt according to claim wherein R⁵ is methyl.

56. The compound or pharmaceutically acceptable salt according to claim 21, wherein Ar²¹ is

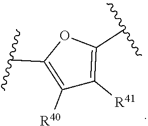

57. The compound or pharmaceutically acceptable salt according to claim 21, wherein Ar²¹ is

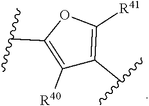

58-66. (canceled)

67. The compound or pharmaceutically acceptable salt according to claim 21, wherein Ar²² is

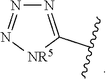

68. The compound or pharmaceutically acceptable salt according to claim 21, wherein Ar²² is

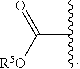

69-70. (canceled)

71. The compound or pharmaceutically acceptable salt according to claim 21, wherein Ar²² is

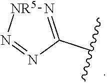

72-77. (canceled)

78. The compound or pharmaceutically acceptable salt according to claim 21, wherein the compound has a structure according to any one of the following formulas

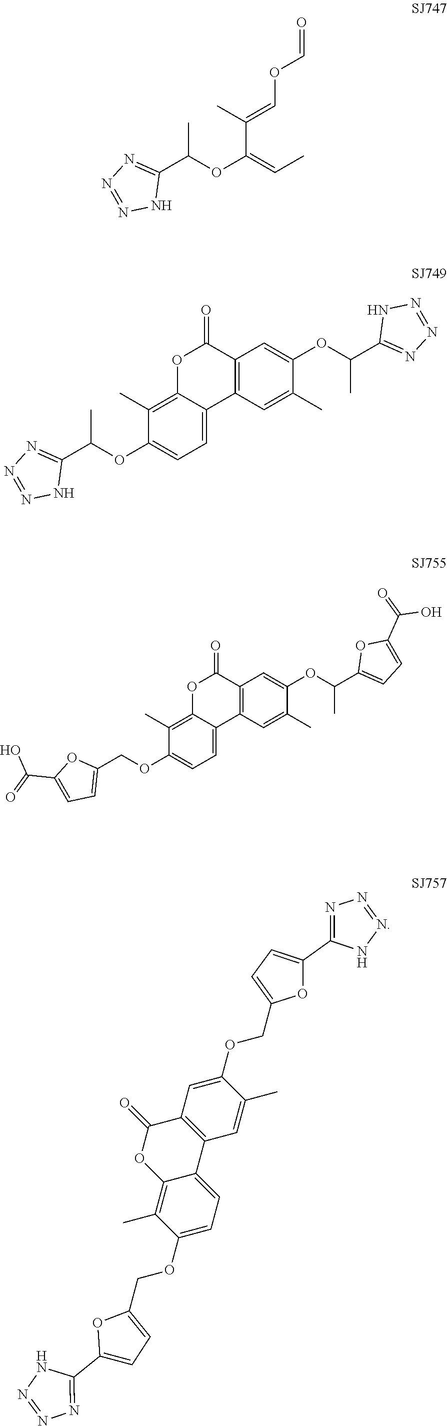 [SMILES: C/C=C(OC(C)c1nnn[nH]1)\C(C)=C\OC=O ; Cc1cc2c(cc1OC(C)c1nnn[nH]1)c(=O)oc1c(C)c(OC(C)c3nnn[nH]3)ccc12 ; Cc1cc2c(cc1OC(C)c1ccc(C(=O)O)o1)c(=O)oc1c(C)c(OCc3ccc(C(=O)O)o3)ccc12 ; Cc1cc2c(cc1OCc1ccc(-c3nnn[nH]3)o1)c(=O)oc1c(C)c(OCc3ccc(-c4nnn[nH]4)o3)ccc12]

79. A pharmaceutical formulation comprising a therapeutically effective amount of a compound or pharmaceutically acceptable salt according to claim 1 and a pharmaceutically acceptable carrier.

80. The pharmaceutical formulation according to claim 79, wherein the compound has a structure according to any one of the following formulas

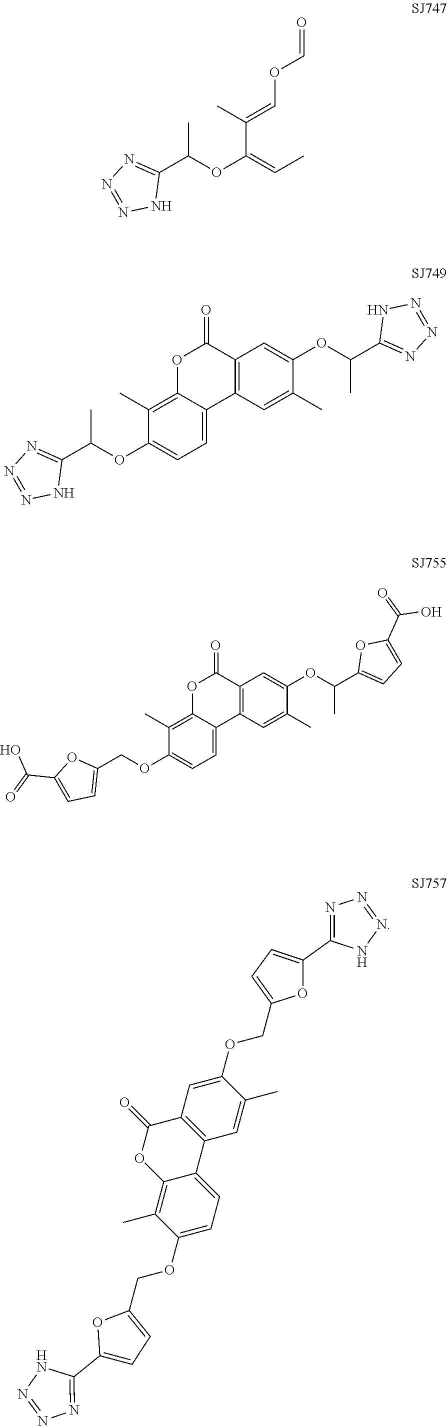 [SMILES: C/C=C(OC(C)c1nnn[nH]1)\C(C)=C\OC=O ; Cc1cc2c(cc1OC(C)c1nnn[nH]1)c(=O)oc1c(C)c(OC(C)c3nnn[nH]3)ccc12 ; Cc1cc2c(cc1OC(C)c1ccc(C(=O)O)o1)c(=O)oc1c(C)c(OCc3ccc(C(=O)O)o3)ccc12 ; Cc1cc2c(cc1OCc1ccc(-c3nnn[nH]3)o1)c(=O)oc1c(C)c(OCc3ccc(-c4nnn[nH]4)o3)ccc12]

81-85. (canceled)

86. A method for the treatment of a disease or disorder associated with expression of intrinsically disordered protein p27 in a subject in need of treatment, the method comprising administering a therapeutically effective amount of a compound or a pharmaceutically acceptable salt according to claim 1.

87-89. (canceled)

90. A method of promoting reentry into the cell division cycle in a subject in need thereof, the method comprising administering a therapeutically effective amount of a compound or a pharmaceutically acceptable salt according to claim 1.

91. (canceled)
